(19) United States
(12) Patent Application Publication
(10) Pub. No.: US 2023/0128229 A1
(43) Pub. Date: Apr. 27, 2023

(54) ENDOSCOPIC TREATMENT TOOL

(71) Applicant: OLYMPUS MEDICAL SYSTEMS CORP., Tokyo (JP)

(72) Inventors: Shigeru MATSUNO, Hachioji-shi (JP); Tatsuya SUZUKI, Hachioji-shi (JP); Tomotaka HAYAKAWA, Kawaguchi-shi (JP); Koji ONISHI, Hachioji-shi (JP); Kazuya YAMANAKA, Hachioji-shi (JP)

(73) Assignee: OLYMPUS MEDICAL SYSTEMS CORP., Tokyo (JP)

(21) Appl. No.: 17/971,897

(22) Filed: Oct. 24, 2022

Related U.S. Application Data
(60) Provisional application No. 63/271,298, filed on Oct. 25, 2021.

(51) Int. Cl. A61B 17/29; A61B 10/04; A61B 10/06

(52) U.S. Cl. CPC A61B 17/29; A61B 10/04; A61B 10/06; A61B 2017/0034

(57) ABSTRACT

An endoscope treatment tool includes: a forceps having a first forceps piece and a second forceps piece; and a joint. The second forceps piece includes a forceps cup, a first tooth, a second tooth, a third tooth, a fourth tooth. A length from a tip of the first tooth to a tip of the second tooth in a longitudinal direction defines a first length, a length from the tip of the first tooth to a tip of the third tooth in the longitudinal direction defines a second length, and the first length is shorter than the second length. A length from the tip of the first tooth to a tip of the fourth tooth in the longitudinal direction defines a third length and the second length is shorter than the third length.

### RELATED APPLICATION DATA

This application based upon and claims the benefit of priority from U.S. Provisional Patent Application Ser. No. 63/271,298, filed on Oct. 25, 2021, the entire content of which is incorporated herein by reference.

### FIELD OF THE DISCLOSURE

The present disclosure relates to an endoscopic treatment tool.

### BACKGROUND

Conventionally, endoscopic treatment tools such as biopsy forceps have been used in endoscopic treatment. Since endoscopic treatment tools such as biopsy forceps are disposable products (throw-away products), they are desired to have high performance and low cost.

Japanese Unexamined Patent Application, First Publication No. H05-212044 (hereinafter referred to as Patent Document 1) and Japanese Unexamined Patent Application, First Publication No. H07-328012 (hereinafter referred to as Patent Document 2) describe biopsy forceps used for endoscopic treatment.

The biopsy forceps according to Patent Document 1 has a treatment portion attached to the distal end portion of the resin sheath and an operation wire for operating the treatment portion. The treatment portion has a pair of cup members arranged so as to face each other, and when the operation wire is pushed and pulled, the pair of cup members are opened and closed to collect tissue. A flat surface is formed on each of the end faces of the pair of cup members on the opposite side.

The biopsy forceps according to Patent Document 2 has a needle provided between a pair of biopsy cups, and collects the tissue in a state where the needle exposed by opening the biopsy cup is pierced into the tissue to be targeted. The needle extends along the central axis of the biopsy forceps.

### SUMMARY OF DISCLOSURE

An endoscope treatment tool includes: a forceps having a first forceps piece and a second forceps piece; and a joint. The second forceps piece includes a forceps cup, a first tooth, a second tooth, a third tooth, a fourth tooth. A length from a tip of the first tooth to a tip of the second tooth in a longitudinal direction defines a first length, a length from the tip of the first tooth to a tip of the third tooth in the longitudinal direction defines a second length, and the first length is shorter than the second length. A length from the tip of the first tooth to a tip of the fourth tooth in the longitudinal direction defines a third length and the second length is shorter than the third length.

### BRIEF DESCRIPTION OF THE DRAWINGS

FIG. 1 is an overall view of an endoscopic treatment system.

FIG. 2 is an overall view showing an endoscopic treatment tool.

FIG. 3 is a perspective view of the distal end of the treatment tool according to a first embodiment.

FIG. 4 is a side view of the distal end of the treatment tool according to the first embodiment.

FIG. 5 is a cross-sectional view of the sheath taken along the line X-X shown in FIG. 2.

FIG. 6 is an exploded perspective view of a treatment portion according to the first embodiment.

FIG. 7 is a perspective view of a frame according to the first embodiment.

FIG. 8 is a front view showing forceps according to the first embodiment.

FIG. 9 is a side view showing the forceps according to the first embodiment.

FIG. 10 is a perspective view of a forceps piece according to the first embodiment.

FIG. 11 is a perspective view of a forceps piece according to the first embodiment.

FIG. 12 is a perspective view of the distal end of the forceps of a modification 1-0 in the treatment portion.

FIG. 13 is a front view of the forceps of the modification 1-0 in the treatment portion.

FIG. 14 is a rear view of the forceps of the modification 1-0 in the treatment portion.

FIG. 15 is a right side view of the forceps of the modification 1-0 in the treatment portion.

FIG. 16 is a left side view of the forceps of the modification 1-0 in the treatment portion.

FIG. 17 is a plan view of the forceps of the modification 1-0 in the treatment portion.

FIG. 18 is a bottom view of the forceps of the modification 1-0 in the treatment portion.

FIG. 19 is a schematic view of the forceps of the modification 1-0 in the treatment portion.

FIG. 20 is a right side view showing the forceps of a modification 1-1 in the treatment portion.

FIG. 21 is a diagram showing forceps of a modification 1-2 in the treatment portion.

FIG. 22 is a bottom view showing a forceps piece of the modification 1-2 in the treatment portion.

FIG. 23 is a perspective view showing the forceps of a modification 1-3 in the treatment portion.

FIG. 24 is a front view showing the forceps of the modification 1-3 in the treatment portion.

FIG. 25 is a rear view showing the forceps of the modification 1-3 in the treatment portion.

FIG. 26 is a right side view showing the forceps of the modification 1-3 in the treatment portion.

FIG. 27 is a left side view showing the forceps of the modification 1-3 in the treatment portion.

FIG. 28 is a plan view showing the forceps of the modification 1-3 in the treatment portion.

FIG. 29 is a bottom view showing the forceps of the modification 1-3 in the treatment portion.

FIG. 30 is a partially enlarged view showing a closed state of the forceps of the modification 1-3.

FIG. 31 is a partially enlarged view showing an open state of the forceps of the modification 1-3.

FIG. 32 is a perspective view showing forceps of the treatment portion according to a second embodiment.

FIG. 33 is a front view showing forceps of the treatment portion according to the second embodiment.

FIG. 34 is a rear view showing forceps of the treatment portion according to the second embodiment.

FIG. 35 is a right side view showing the forceps of the treatment portion according to the second embodiment.

FIG. 36 is a left side view showing the forceps of the treatment portion according to the second embodiment.

FIG. 37 is a plan view showing forceps of the treatment portion according to the second embodiment.

FIG. 38 is a bottom view showing forceps of the treatment portion according to the second embodiment.

FIG. 39 is a partially enlarged perspective view of the forceps according to the second embodiment, and is an enlarged view showing the forceps of the endoscopic treatment tool according to the second embodiment.

FIG. 40 is a partially enlarged perspective view of another embodiment of the forceps according to the second embodiment.

FIG. 41 is a partial cross-sectional view showing forceps of the endoscopic treatment tool according to the second embodiment.

FIG. 42 is a diagram showing a state in which the tissue is grasped by the forceps according to the second embodiment.

FIG. 43 is a perspective view of the forceps of the modification 2-1 in the treatment portion as viewed from the distal end side.

FIG. 44 is a partial cross-sectional view of the forceps of the modification 2-1 in the treatment portion.

FIG. 45 is a partial cross-sectional view showing the forceps of the modification 2-1 in the treatment portion.

FIG. 46 is a bottom view showing a forceps piece of the forceps of the modification 2-1 in the treatment portion.

FIG. 47 is a right side view partially showing the forceps of the modification 2-3 in the treatment portion.

FIG. 48 is a perspective view showing an open state of the forceps in the treatment portion according to a third embodiment.

FIG. 49 is a side view showing an open state of forceps in the treatment portion according to the third embodiment.

FIG. 50 is a side view showing a closed forceps in the treatment portion according to the third embodiment.

FIG. 51 is a plan view showing the closed forceps in the treatment portion according to the third embodiment.

FIG. 52 is a view showing forceps (forceps pieces) in the treatment portion according to the third embodiment, and is a plan view in which the forceps pieces are not shown.

FIG. 53 is a front view of the closed forceps in the treatment portion according to the third embodiment.

FIG. 54 is a front view of the open forceps in the treatment portion according to the third embodiment.

FIG. 55 is a schematic diagram showing an approach posture of the treatment portion with respect to a target tissue T.

FIG. 56 is a perspective view showing a closed state of forceps 5Ca of the modification 3-1 in the treatment portion.

FIG. 57 is a front view showing a closed state of the forceps of the modification 3-1 in the treatment portion.

FIG. 58 is a rear view showing a closed state of the forceps of the modification 3-1 in the treatment portion.

FIG. 59 is a right side view showing a closed state of the forceps of the modification 3-1 in the treatment portion.

FIG. 60 is a left side view showing a closed state of the forceps of the modification 3-1 in the treatment portion.

FIG. 61 is a plan view showing a closed state of the forceps of the modification 3-1 in the treatment portion.

FIG. 62 is a bottom view showing a closed state of the forceps of the modification 3-1 in the treatment portion.

FIG. 63 is a view showing the forceps (forcer pieces) of the modification 3-1 in the treatment portion, and is a plan view showing the forceps pieces.

FIG. 64 is a perspective view showing the forceps of the modification 3-1 in the treatment portion.

FIG. 65 is a right side view showing the forceps of the modification 3-1 in the treatment portion.

FIG. 66 is an overall view of the endoscopic treatment tool according to a fourth embodiment.

FIG. 67 is a cross-sectional view of an operating portion of the endoscopic treatment tool in an open state.

FIG. 68 is a cross-sectional view of the operating portion of the endoscopic treatment tool in a closed state.

FIG. 69 is a perspective view of the slider seen from below in the operating portion.

FIG. 70 is a cross-sectional view of the operating portion equipped with a rubber dome.

FIG. 71 is a diagram showing a rubber dome.

FIG. 72 is a perspective view of a slider viewed from below in an operating portion equipped with a leaf spring.

FIG. 73 is a perspective view of forceps of an endoscopic treatment instrument according to a fifth embodiment.

FIG. 74 is a right side view of the forceps.

FIG. 75 is a left side view of the forceps.

FIG. 76 is a front view of the forceps.

FIG. 77 is a perspective view of a second forceps piece of the forceps.

FIG. 78 is a side view of the second forceps piece.

FIG. 79 is a front view of the second forceps piece.

FIG. 80 is a plan view of the second forceps piece.

FIG. 81 is a perspective view of a first forceps piece of the forceps.

FIG. 82 is a cross-sectional view of the forceps gripping a living tissue.

FIG. 83 is a cross-sectional view of the forceps gripping a living tissue.

FIG. 84 is a perspective view of the forceps of an endoscopic treatment instrument according to a sixth embodiment.

FIG. 85 is a right side view of the forceps.

FIG. 86 is a left side view of the forceps.

FIG. 87 is a front view of the forceps.

FIG. 88 is a perspective view of a second forceps piece of the forceps.

FIG. 89 is a side view of the second forceps piece.

FIG. 90 is a front view of the second forceps piece.

FIG. 91 is a plan view of the second forceps piece.

FIG. 92 is a perspective view of a first forceps piece of the forceps.

FIG. 93 is a plan view of the second forceps piece.

FIG. 94 is a right side view of the forceps gripping a living tissue.

FIG. 95 is a left side view of the forceps gripping a living tissue.

FIG. 96 is a cross-sectional view along X2 shown in FIGS. 93 to 95.

FIG. 97 is a sectional view along X3 shown in FIGS. 93 to 95.

FIG. 98 is a sectional view along X4 shown in FIGS. 93 to 95.

### DETAILED DESCRIPTION

### First Embodiment

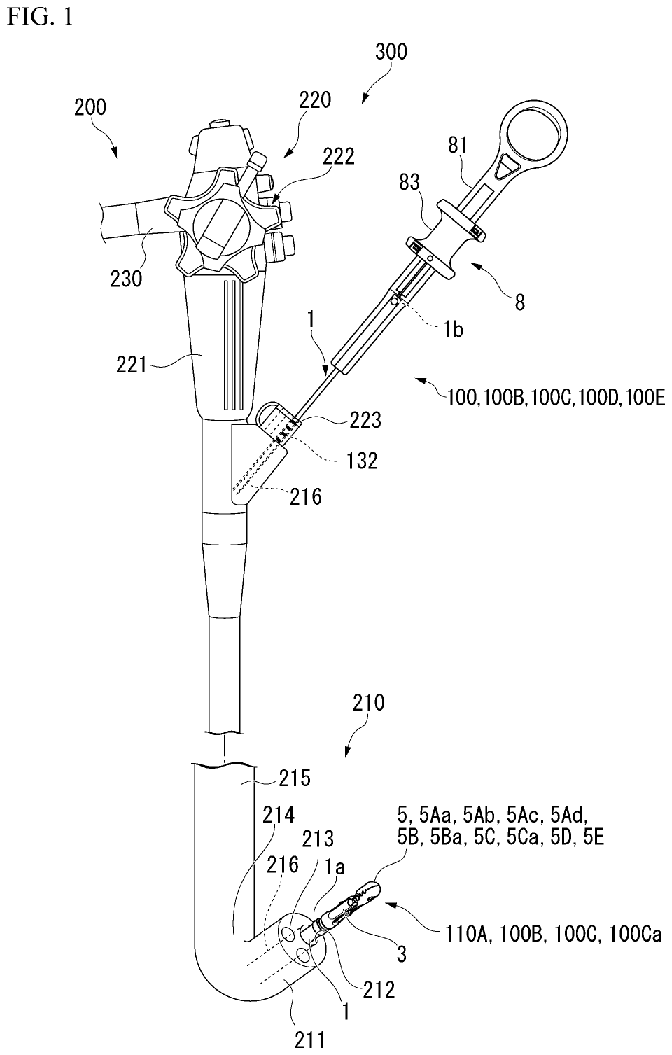

An endoscopic treatment system 300 including an endoscopic treatment tool 100 according to a first embodiment of the present disclosure will be described with reference to FIGS. 1 to 11. In each of the drawings below, the scale of dimensions may differ depending on the components in order to make each component easy to see.

FIG. 1 is an overall view of the endoscopic treatment system 300.

[Endoscopic treatment system 300]

As shown in FIG. 1, the endoscopic treatment system 300 includes the endoscopic treatment tool 100 and an endoscope 200. The endoscopic treatment tool 100 is used by being inserted into the endoscope 200.

### [Endoscope 200]

The endoscope 200 is a known flexible endoscope, and includes an insertion portion 210 that is inserted into the body from the distal end, an operating portion 220 that is attached to the proximal end of the insertion portion 210, and a universal cord 230 attached to the operating portion 220.

The insertion portion 210 is an elongated long member that can be inserted into the lumen. The insertion portion 210 has a distal end portion 211, a curved portion 214, and a soft portion 215. The distal end portion 211, the curved portion 214, and the soft portion 215 are connected in order from the distal end side. A channel 216 for inserting the treatment tool 100 is provided inside the insertion portion 210. The distal end portion 211 is provided with a distal end opening portion 212 of the channel 216 and an imaging portion 213.

The imaging portion 213 is provided with an imaging element such as a CCD or CMOS, and can image a portion to be treated. The curved portion 214 is curved according to the operation of the operating portion 220 by the user. The flexible portion 215 is a flexible tubular portion.

The operating portion 220 is connected to the soft portion 215. The operating portion 220 has a grip 221 and an input portion 222, and a forceps opening 223. The grip 221 is a member supported by the user. The input portion 222 receives an operation input for bending the curved portion 214. The forceps opening 223 is a proximal opening of the channel 216.

The universal cord 230 connects the endoscopic treatment tool 100 and an external device. An imaging cable, an optical fiber cable, or the like that outputs an imaging signal imaged by the imaging portion 213 to the outside is inserted in the universal cord 230.

[Endoscopic treatment tool 100]

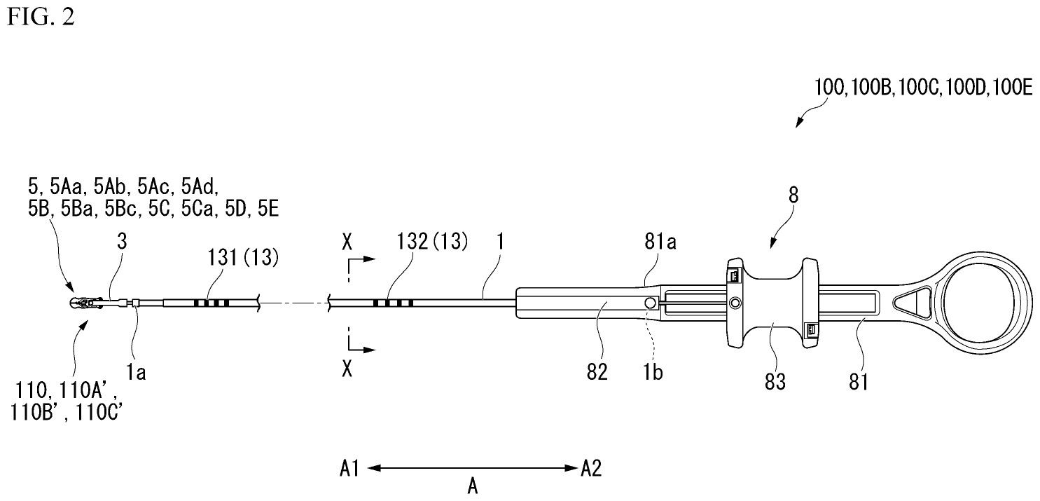

FIG. 2 is an overall view showing the endoscopic treatment tool 100.

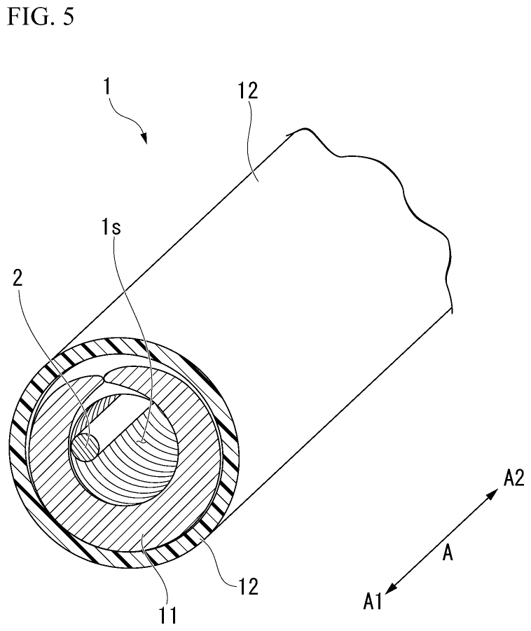

The endoscopic treatment tool 100 (also referred to as the treatment tool 100) includes a sheath 1, an operation wire 2 (see FIG. 5), a support member (cover member, rotatable joint) 3, forceps (jaw) 5, and an operating portion 8. In the following description, as shown in FIG. 2, in the longitudinal direction A along the central axis of the treatment tool 100, the side inserted into the patient's body is referred to as a “distal end side A1” and the side of the operating portion 8 is referred to as a “proximal end side A2”.

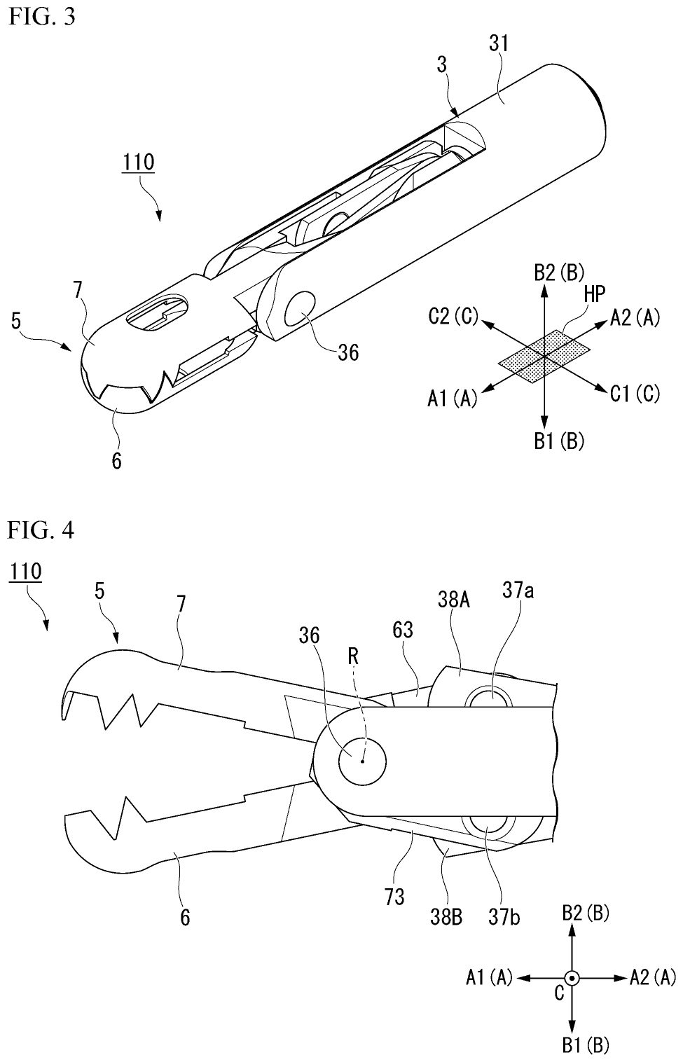

FIG. 3 is a perspective view of the distal end of the treatment tool 100. FIG. 4 is a side view of the distal end portion of the treatment tool 100.

As shown in FIG. 3, a support member 3 and forceps 5 is provided at the distal end of the treatment tool 100. The forceps 5 is supported by a support member 3 which is a link mechanism so as to be capable of opening and closing. The support member 3 and the forceps 5 form a “treatment portion 110” for treating the affected portion.

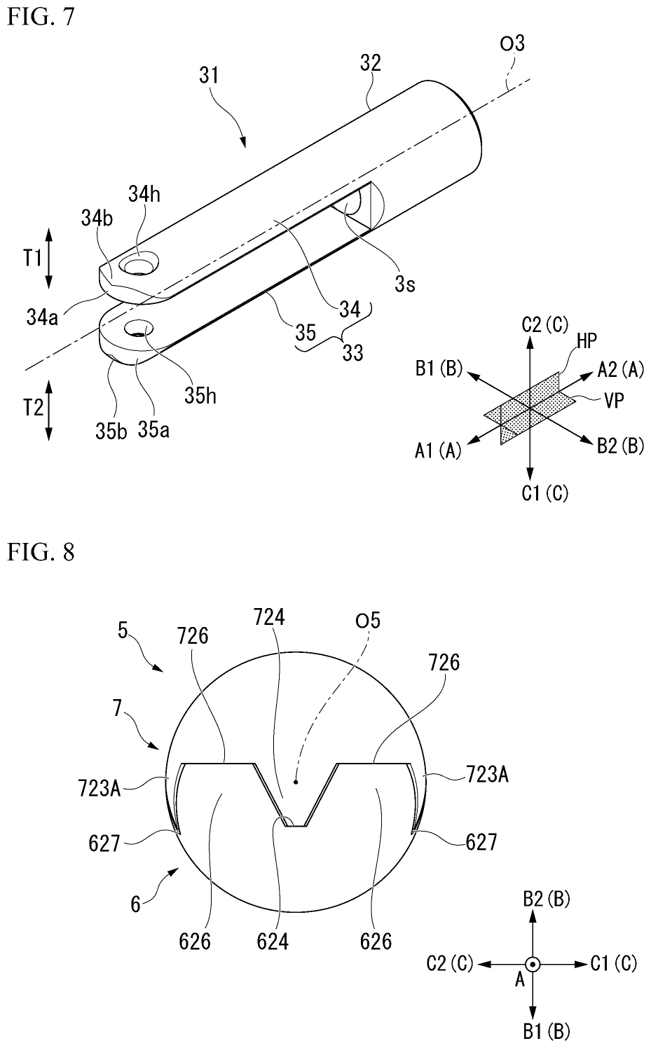

In the following description, as shown in FIG. 3, the direction in which the forceps 5 opens and closes is referred to as an “opening/closing direction B” or a “vertical direction B”. Further, the direction perpendicular to the longitudinal direction A and the opening/closing direction B is referred to as a “width direction C” or a “left-right direction C”. Further, the plane horizontal to the longitudinal direction A and the width direction C is referred to as a “horizontal plane HP”. As shown in FIG. 7, a plane horizontal to the longitudinal direction A and the opening/closing direction B is referred to as a “vertical plane VP”.

In the following description, the opening direction of the forceps piece 6 will be referred to as a “lower side B1” in the opening/closing direction B, and the opening direction of the forceps piece 7 will be referred to as an “upper side B2” in the opening/closing direction B. Further, the direction facing right from the distal end side A1 to the proximal end side A2 is defined as a “right side C1” in the width direction C, and the direction facing left is defined as a “left side C2” in the width direction C.

### [Sheath 1]

The sheath 1 is a long member that has flexibility and extends from a distal end 1a to a proximal end 1b, as shown in FIGS. 1 and 2. The sheath 1 has an outer diameter that can be inserted into the channel 216 of the endoscope 200. As shown in FIG. 1, in a state where the sheath 1 is inserted into the channel 216, the distal end 1a of the sheath 1 can be recessed from the distal end opening 212 of the channel 216. The sheath 1 may have an insulating property.

FIG. 5 is a cross-sectional view of the sheath 1 along the X-X line shown in FIG. 2.

The sheath 1 includes a coil sheath 11 formed by spirally winding a metal wire. A tube sheath made of resin is provided in an internal space 1s of the sheath 1. The operation wire 2 is inserted through the inside of the tube sheath. With this configuration, the friction between the operation wire 2 and the coil sheath 1 can be reduced. However, this embodiment is not limited to this, and there may be a covering tube 12 that covers the outer peripheral surface of the coil sheath 11 and a marking 13 provided on the outer surface of the covering tube 12.

As shown in FIG. 2, the marking 13 includes a first marking 131 provided near the distal end 1a of the sheath 1 and a second marking 132 provided near the proximal end 1b of the sheath 1.

The first marking 131 is provided at a position close to the treatment portion 110 on the distal end side A1. The user can recognize that the treatment portion 110 is approaching the forceps opening 223 by checking the first marking 131 when the treatment tool 100 is removed from the channel 216 of the endoscope 200. Therefore, the user can slowly discharge the treatment portion 110 that grasps the living body or the like from, for example, the forceps opening 223.

The second marking 132 is provided on the proximal end side A2 and at a position close to the operating portion 8. Specifically, as shown in FIG. 1, the second marking 132 is provided at a position where the treatment portion 110 is inserted into the channel 216 from the forceps opening 223 when the treatment portion 110 protrudes from the distal end opening 212 of the endoscope 200. By confirming the position of the second marking 132 with respect to the forceps opening 223, the user can recognize the position of the treatment portion 110 with respect to the distal end opening 212. Therefore, the user can slowly protrude the treatment portion 110 from, for example, the distal end opening 212 of the endoscope 200.

[Operation wire 2]

The operation wire 2 is a metal wire through which the internal space is of the sheath 1 is inserted. The distal end of the operation wire 2 is connected to the forceps 5, and the proximal end of the operation wire 2 is connected to the operating portion 8.

As shown in FIG. 5, the operation wire 2 is inserted through the internal space 1s of the sheath 1.

[Support member 3]

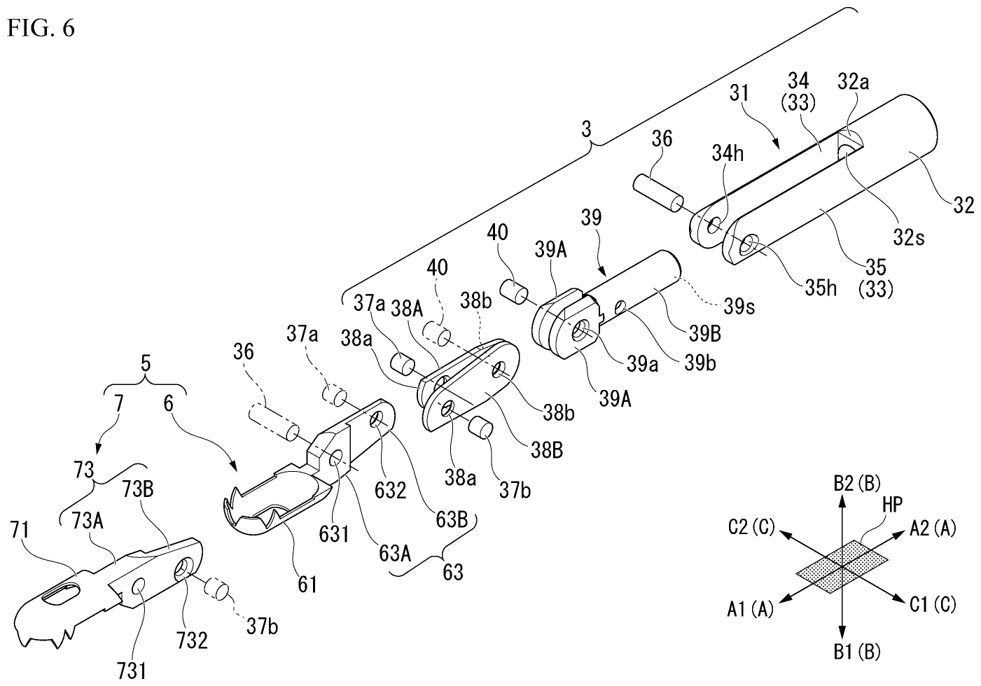

FIG. 6 is an exploded perspective view of the treatment portion 110.

As shown in FIG. 1, the support member 3 has a proximal end side A2 attached to the distal end 1a of the sheath 1 and supports the forceps 5 on the distal end side A1. The support member 3 includes a frame 31, a forceps opening/closing pin (rotation shaft, rotation shaft member) 36, gripping piece connecting pins 37a and 37b, a pair of support plate members 38A and 38B, a connecting portion 39, and a support plate connecting pin 40. As shown in FIG. 3, the forceps 5 is connected by the forceps opening/closing pin 36 attached to the frame 31 so as to be capable of opening and closing around the axis of the forceps opening/closing pin 36.

FIG. 7 is a perspective view of the frame 31.

The frame 31 is formed of a metal such as stainless steel into a substantially U-shape. The frame 31 has a support main body 32 formed in a cylindrical shape and a pair of frame pieces 33 (first frame piece 34 and second frame piece 35).

The support body 32 is fixed to the distal end 1a of the sheath 1 by caulking or the like. The central axis O3 in the longitudinal direction A of the support member 3 substantially coincides with the central axis O1 in the longitudinal direction A of the sheath 1. An internal space 3s (FIG. 7) of the support body 32 communicates with the internal space is (FIG. 5) of the sheath 1. The operation wire 2 is inserted in the internal space 3s of the support portion main body 32.

As shown in FIG. 7, the pair of frame pieces 33 (first frame piece 34 and second frame piece 35) are provided so as to protrude from the support portion main body 32 to the distal end side A1. The first frame piece 34 and the second frame piece 35 are evenly provided on both sides in the vertical direction B with the central axis O3 interposed therebetween. The first frame piece 34 and the second frame piece 35 have a shape symmetrical with respect to the vertical plane VP passing through the central axis O3.

The first frame piece 34 is formed in a flat plate shape extending in the longitudinal direction A. The plate thickness direction T1 of the first frame piece 34 substantially coincides with the vertical direction B. The proximal end side A2 of the first frame piece 34 is connected to the support main body 32. A semicircular first distal end portion 34a is formed on the distal end side A1 of the first frame piece. An inclined side 34b is partially formed on the outside of the first distal end portion 34a in the width direction C. The thickness of the first frame piece 34 in the plate thickness direction T1 is constant in the longitudinal direction A.

A first through-hole 34h penetrating in the plate thickness direction T1 is formed on the distal end side A1 of the first frame piece 34.

The second frame piece 35 is formed in a flat plate shape extending in the longitudinal direction A. The plate thickness direction T2 of the second frame piece 35 substantially coincides with the vertical direction B. The proximal end side A2 of the second frame piece 35 is connected to the support main body 32. A semicircular second distal end portion 35a is formed on the distal end side A1 of the second frame piece 35. An inclined side 35b is formed on the outside of the second distal end portion 35a in the width direction C.

The thickness of the second frame piece 35 in the plate thickness direction T2 is the same as the thickness of the first frame piece 34 in the plate thickness direction T1.

A second through-hole 35h penetrating in the plate thickness direction T2 is formed on the distal end side A1 of the second frame piece 35. The center of the second through-hole 35h coincides with the center of the first through-hole 34h.

As shown in FIG. 6, the forceps opening/closing pin (rotation shaft, rotation shaft member) 36 is formed in a substantially cylindrical shape by a metal such as stainless steel. As shown in FIG. 6, the forceps opening/closing pin 36 engages with the first through-hole 34h of the first frame piece 34 and the second through-hole 35h of the second frame piece 35 and is attached to the frame 31.

The pair of support plate members 38A and 38B are formed in a plate shape by a metal such as stainless steel. The pair of support plate members 38A and 38B have the same shape and are stacked in the plate thickness direction in an upside-down posture. In the support plate members 38A and 38B, a first through-hole 38a is formed on the distal end side A1 and a second through-hole 38b is formed on the proximal end side A2. The pair of support plate members 38A and 38B are overlapped with each other in a state where the second through-holes 38b on the proximal end side A2 communicate with each other and the first through-holes 38a are displaced in the vertical direction (opening/closing direction of the forceps 5).

The support plate member 38A is connected to the forceps piece (second gripping piece) 6 of the forceps 5 via the gripping piece connecting pin 37a inserted into the first through-hole 38a. The support plate member 38B is connected to the forceps piece (first gripping piece) 7 of the forceps 5 via the gripping piece connecting pin 37b inserted into the first through-hole 38a. These support plate members 38A and 38B are connected to the connecting portion 39 via the support plate connecting pins 40 inserted into the second through-holes 38b of each other, and are rotatably supported around the axis of the support plate connecting pins 40.

The connecting portion 39 is coaxially arranged in the support portion main body 32 (FIG. 14). The connecting portion 39 has a pair of connecting pieces 39A located on the distal end side A1 and a tubular body 39B connected to the proximal end side A2 of the connecting pieces 39A. An internal space 39s is formed in the tubular body 39B, the distal end side A1 is closed, and the proximal end side A2 is open. A through-hole 39b communicating with the internal space 39s from the right side C1 is formed on the distal end side A1 of the tubular body 39B. The through-hole 39b does not penetrate the left side C2 of the tubular body 39B.

The pair of connecting pieces 39A are provided in parallel with an interval in the width direction C. The pair of support plate members 38A and 38B are inserted between the pair of connecting pieces 39A. A through-hole 39a penetrating in the plate thickness direction is formed in the center of each connecting piece 39A. The through-holes 39a are concentric circles and communicate with each other. The connecting portion 39 rotatably supports the support plate members 38A and 38B via the support plate connecting pins 40 inserted into the through-holes 39a formed in the pair of connecting pieces 39A.

Of the connecting portions 39, the tubular body 39B is inserted from the proximal end side A2 of the frame 31 into the internal space 32s of the support portion main body 32, and the pair of connecting pieces 39A are locked to the distal end surface 32a of the support portion main body 32. The operation wire 2 is connected to the tubular body 39B of the connecting portion 39. Further, a coil sheath 11 is connected to the support portion main body 32.

### [Forceps (Jaw) 5]

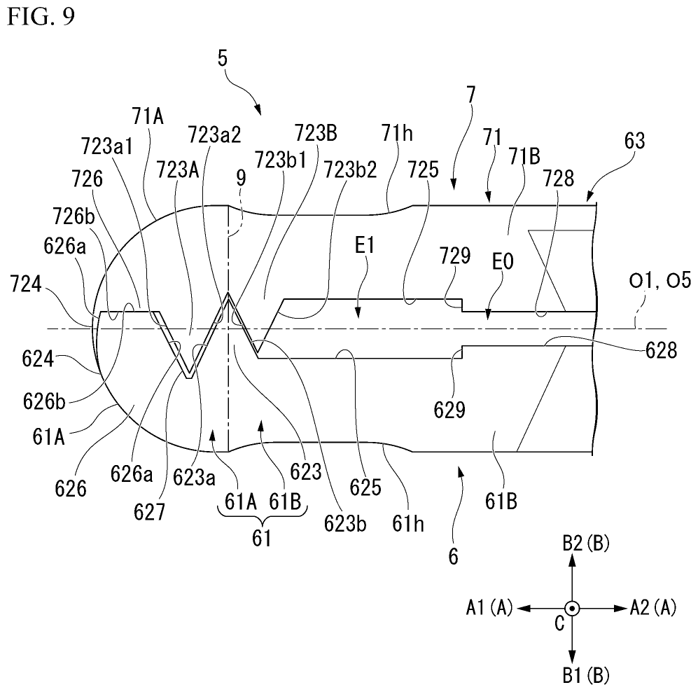

FIG. 8 is a front view showing the forceps 5. FIG. 9 is a side view showing the forceps 5.

The forceps (jaw) 5 is a member for collecting living tissue. As shown in FIGS. 4 and 8, the forceps 5 is made of a metal material such as stainless steel, and include forceps pieces 6 and forceps 7 that face each other in the closed state. When the forceps piece 6 and the forceps piece 7 are in the closed state, the forceps 5 has a tubular shape.

The forceps piece 6 is rotatably supported by the pin 36 with the rotation axis R of the pin 36 shown in FIG. 4 as the center of rotation.

The forceps piece 7 is rotatably supported by the pin 36 with the rotation axis R of the pin 36 shown in FIG. 4 as the center of rotation.

The proximal end side A2 of each of these forceps pieces 6 and 7 is connected to the support plate members 38A and 38B. Further, the support plate members 38A and 38B are connected to the connecting portion 39 and operated by the operation wire 2 (FIG. 5) connected to the connecting portion 39.

The forceps piece 6 and the forceps piece 7 are arranged so as to be symmetrical with respect to the central axis O5 in the longitudinal direction A of the forceps 5. The central axis O5 in the longitudinal direction A of the forceps 5 substantially coincides with the central axis O1 in the longitudinal direction A of the sheath 1.

Note that one of the forceps piece 6 and the forceps piece 7 may be fixed to the support member 3 and only the other may be rotatably supported by the support member 3.

[Forceps piece 6]

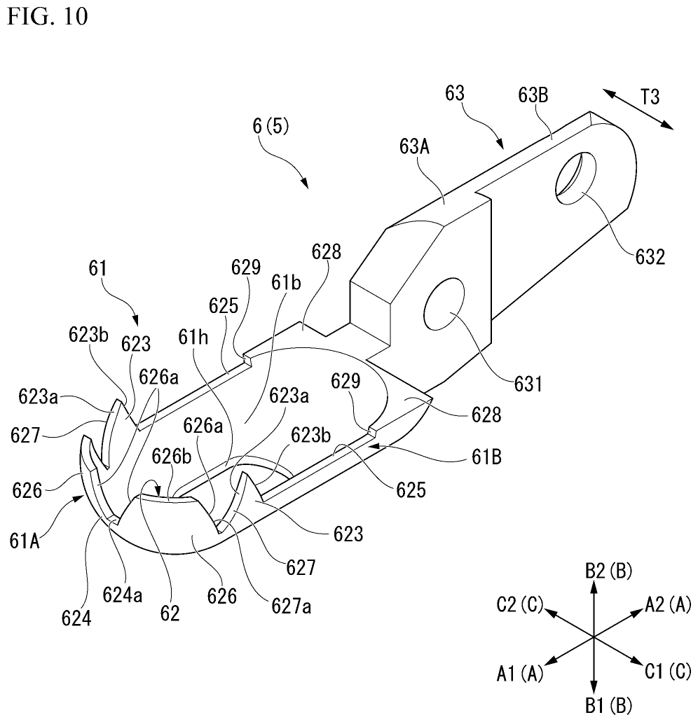

FIG. 10 is a perspective view of the forceps piece 6.

The forceps piece (second gripping piece) 6 is formed from, for example, one flat plate or one bar member mainly by cutting or pressing. As shown in FIGS. 8, 9, and 10, the forceps piece 6 includes a forceps cup (second forceps cup) 61 provided on the distal end side A1 in the longitudinal direction A, and a first plate 63.

The forceps cup 61 is formed in a substantially hemispherical shape as shown in FIG. 10, and opens toward the side of the forceps piece 7 (upper side B2) shown in FIG. 9 in the opening/closing direction B. The forceps cup 61 includes an opening edge 62 and a through-hole 61h penetrating in the opening/closing direction B near the center of the concave bottom portion 61b.

As shown in FIG. 10, the opening edge 62 of the forceps cup 61 includes a distal end groove portion 624, a pair of protruding portions 626, a pair of side groove portions 627, a pair of protruding teeth 623, a pair of first flat portions 625 (flat surface), a pair of second flat portions 628, and a pair of stepped portions 629, and has an uneven shape in the vertical direction (opening/closing direction) B.

As shown in FIGS. 8 and 9, the distal end groove portion 624 is located on the central axis O5 and is located on the most distal end side A1 of the distal end portion 61A of the forceps cup 61. The distal end groove portion 624 is formed between a pair of protruding portions 626, and has a substantially triangular shape in which the shape seen from the distal end side A1 in FIG. 8 is recessed in the lower side B1. As shown in FIGS. 8 and 10, the distal end groove portion 624 is formed by a base 624a, which is formed of a plane perpendicular to the central axis O5, and a pair of hypotenuses 626a, which extends both-outside in the width direction C from the base 624a toward the upper side B2.

The pair of protruding portions 626 are located on both sides of the distal end groove portion 624 in the distal end portion 61A of the forceps cup 61. The pair of protruding portions 626 protrude at the same height toward the upper side B2, and the shape seen from the distal end side A1 forms a trapezoidal shape. The end side 626b of the protruding portion 626 is a surface parallel to the base 624a of the distal end groove portion 624, and the shape seen from the upper side B2 forms an arcuate shape. As shown in FIG. 9, the end side 626b is located closer to the forceps piece 7 (upper side B2) than the central axis O5. The pair of protruding teeth 623 are provided on the proximal end side A2 of each protruding portion 626 via the side groove portion 627.

The pair of protruding teeth 623 protrude to the upper side B2 as shown in FIGS. 9 and 10. Each protruding tooth 623 has hypotenuses 623a and 623b that approach each other toward the tooth tip (upper side B2), and the shape seen from the width direction C is substantially triangular. The protruding tooth 623 is formed near the boundary between the distal end portion 61A and the semi-cylindrical portion 61B of the forceps cup 61, and the tooth tip is located on the side of the semi-cylindrical portion 61B. The protruding tooth 623 has a higher protruding height than the protruding portion 626, and the tooth tip protrudes to the upper side B2 (the side of the forceps piece 7) of the central axis O5 and the pair of protruding portions 626.

The pair of side groove portions 627 are formed between the protruding portions 626 located on both sides in the width direction C and the protruding tooth 623. Each side groove portion 627 has a substantially triangular shape that is recessed in the lower side B1 when viewed from the width direction C. As shown in FIG. 10, in the vertical direction, the bottom portion 627a of the side groove portion 627 substantially coincides with the position of the base 624a of the distal end groove portion 624. The side groove portion 627 is formed by the hypotenuse 626a of the proximal end side A2 of the protrusion 626 and the hypotenuse 623a of the distal end side A1 of the protruding tooth 623. As described above, since the protruding tooth 623 protrude to the upper side B2 from the protruding portion 626, the distal end side hypotenuse 623a of the protruding tooth 623 has a longer length than the proximal end side hypotenuse 626a of the protruding portion 626.

The pair of first flat portions 625 are formed on the proximal end side A2 of the pair of protruding tooth 623 and extend linearly along the central axis O5. As shown in FIG. 9, when the forceps 5 is closed, the first flat portion 625 faces a first flat portion 725 on the side of the forceps piece 7 in the vertical direction, and a gap E1 is formed between the first flat portion 625 and the first flat portion 725. Since the tissue sandwiched by the forceps 5 can be released from the gap E1 to the outside, a large amount of tissue can be collected.

The pair of second flat portions 628 are located on the proximal end side A2 of the pair of first flat portions 625. The second flat portion 628 is located on B2 (the side of the forceps piece 7) above the first flat portion 625. A part of the semi-cylindrical portion 61B of the forceps cup 61 is stepped by the stepped portion 629 formed between the first flat portion 625 and the second flat portion 628, and the rigidity of the proximal end side A2 of the forceps cup 61 is increased.

As shown in FIG. 10, the first plate 63 is provided on the proximal end side A2 of the forceps cup 61 and is formed in a substantially plate shape. The plate thickness direction T3 of the first plate 63 substantially coincides with the width direction C. As shown in FIG. 6, the first plate 63 is inserted into the frame 31 and is arranged adjacent to the support plate member 38A on the left side C2 of the support plate member 38A. The first plate 63 is rotatably connected to the support plate member 38A by the pin 37a.

The first plate 63 includes a first portion 63A and a second portion 63B.

The first portion 63A is formed on the distal end side A1 of the first plate 63.

The first portion 63A is arranged adjacent to the forceps piece 7 on the right side C1 of the first frame piece 34 of the frame 31 shown in FIG. 6, and is located between the first frame piece 34 and the second plate 73 of the forceps piece 7.

The second portion 63B is formed on the proximal end side A2 of the first portion 63A. The second portion 63B is arranged adjacent to the support plate member 38A on the right side C1 of the first frame piece 34, and is located between the first frame piece 34 and the support plate member 38A. The thickness of the second portion 63B is thinner than that of the first portion 63A in the plate thickness direction T3, and the difference in thickness between the two portions substantially corresponds to the plate thickness of the support plate member 38A.

The first plate 63 includes a first hole 631 and a second hole 632. The first hole 631 is a through-hole that penetrates the first portion 63A in the plate thickness direction T3, and communicates with the first through-hole 34h of the first frame piece 34 shown in FIG. 6. The second hole 632 is a through-hole penetrating the second portion 63B in the plate thickness direction T3, and communicates with the second through-hole 38b of the support plate member 38A shown in FIG. 6.

As shown in FIG. 6, a pin 36 can be inserted into the first hole 631. Further, a pin 37a can be inserted into the second hole 632. The first portion 63A of the forceps piece 6 is rotatably connected to the frame 31 via the pin 36. The second portion 63B of the forceps piece 6 is rotatably connected to the support plate member 38A via the pin 37a.

[Forceps piece 7]

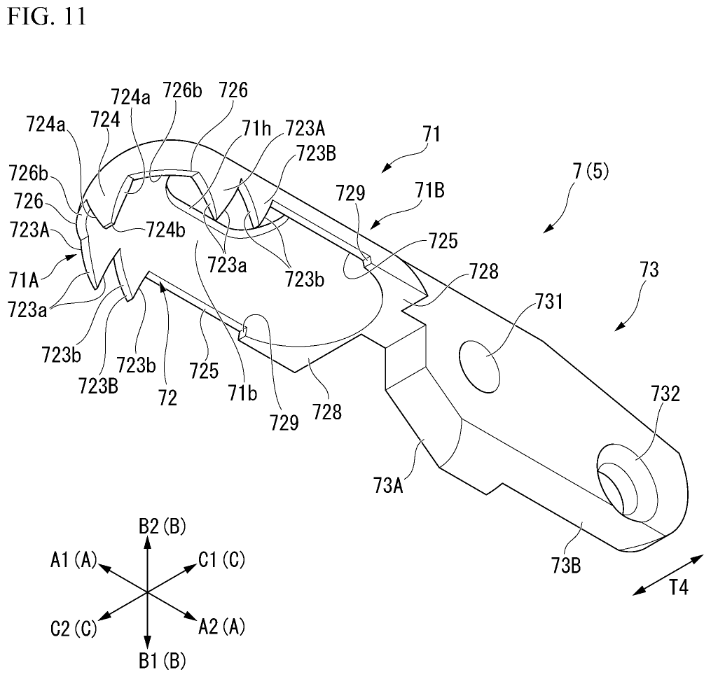

FIG. 11 is a perspective view of the forceps piece 7.

The forceps piece (first gripping piece) 7 is formed from, for example, a single flat plate or one bar member mainly by cutting or pressing. As shown in FIGS. 8, 9, and 11, the forceps piece 7 has a forceps cup (first forceps cup) 71 provided on the distal end side A1 in the longitudinal direction A, and a second plate 73.

The forceps cup 71 is formed in a substantially hemispherical shape as shown in FIG. 11, and opens toward the side of the forceps piece 6 (lower side B1) shown in FIG. 9 in the opening/closing direction B. As shown in FIG. 11, the forceps cup 71 has an opening edge 72 and a through-hole 71h penetrating in the opening/closing direction B near the center of the concave bottom portion 71b.

The opening edge 72 includes a most advanced protruding tooth 724, a pair of recesses 726, a pair of first distal end protruding teeth (first tooth) 723A, a pair of second distal end protruding teeth (second tooth) 723B, a pair of first flat portions 725, a pair of second flat portions 728, and a pair of stepped portions 729, and has an uneven shape in the vertical direction (opening/closing direction).

As shown in FIGS. 8, 9 and 11, the most advanced protruding tooth 724 is located on the central axis O5 and is located on the most distal end side A1 of the distal end 71A of the forceps cup 71. The most advanced protruding tooth 724 protrudes toward the lower side B1 (forceps piece 6) and is housed in the distal end groove portion 624 (FIG. 8) of the forceps piece 6 when the forceps 5 is in the closed state. The most advanced protruding tooth 724 has a pair of hypotenuses 724a, which approach each other toward the tooth tip (lower side B1), an end side 724b, which is formed of a plane horizontal to the central axis O5, and has a substantially triangular shape when viewed from the distal end side A1.

The pair of recesses 726 are located at the distal end 71A of the forceps cup 71, and are formed between the most advanced protruding tooth 724 and the pair of first distal end protruding teeth 723A. The recess 726 is recessed to the upper side B2 and can accommodate the protruding portion 626 of the forceps piece 6 when the forceps 5 is in the closed state. The bottom surface 726b of the recess 726 is a surface facing the end side 626b of the protruding portion 626 (FIG. 10) of the forceps piece 6, and the shape seen from the lower side B1 is arcuate. As shown in FIG. 9, the bottom surface 726b is located on the upper side B2 of the central axis O5. The pair of first distal end protruding teeth 723A are provided on the proximal end side A2 of the pair of recesses 726.

The first distal end protruding tooth 723A protrudes to the lower side B1 as shown in FIGS. 9 and 11. Each first distal end protruding tooth 723A has a hypotenuse 723a1 and a hypotenuse 723a2 that approach each other toward the tooth tip (lower side B1), and the shape seen from the width direction C is substantially triangular. The first distal end protruding tooth 723A is located closer to the distal end 71A than the boundary line 9 with the semi-cylindrical portion 71B in the distal end 71A of the forceps cup 71. The first distal end protruding tooth 723A protrudes toward the side of the forceps piece 6 (lower side B1) from the central axis O5, and the protruding height thereof is substantially equal to the protruding height of the most advanced protruding tooth 724. The protruding height of the most advanced protruding tooth 724 is, for example, 0.3 mm, but is not limited thereto.

The protruding height (diameter length) of the first distal end protruding tooth 723A may be one-third or more of the maximum outer diameter D of the cylindrical shape formed when the forceps piece 6 and the forceps piece 7 are closed. In the present embodiment, the maximum outer diameter D of the forceps 5 is, but is not limited to, about 1 mm. The first distal end protruding tooth 723A is housed in the side groove portion 627 of the forceps piece 6 when the forceps 5 is closed.

The second distal end protruding tooth 723B protrudes to the lower side B1 as shown in FIGS. 9 and 11. The second distal end protruding tooth 723B protrudes toward the lower side B1 less than the first distal end protruding tooth 723A. The second distal end protruding tooth 723B has a hypotenuse 723b1 and a hypotenuse 723b2 that approach each other toward the tooth tip (lower side B1), and the shape seen from the width direction C is substantially triangular. The size of the second distal end protruding tooth 723B is smaller than that of the first distal end protruding tooth 723A.

As shown in FIG. 9, the second distal end protruding tooth 723B is formed in the semi-cylindrical portion 71B of the forceps cup 71, and is arranged on the proximal end side A2 of the first distal end protruding tooth 723A. The pair of first flat portions 725 are provided on the proximal end side A2 of the pair of second distal end protruding teeth 723B.

The pair of first flat portions 725 are formed on the proximal end side A2 of the pair of second distal end protruding teeth 723B, and extend linearly along the central axis O5. When the forceps 5 is closed, the first flat portion 725 faces the first flat portion 625 on the side of the forceps piece 6 in the vertical direction, and a predetermined gap E1 is formed between the first flat portion 725 and the first flat portion 625. Since the tissue sandwiched by the forceps 5 can be released from the gap E1 to the outside, a large amount of tissue can be collected.

The pair of second flat portions 728 are located on the proximal end side A2 of the pair of first flat portions 725. The second flat portion 728 is located on the lower side B1 (the side of the forceps piece 6) of the first flat portion 725. Due to the stepped portion 729 formed at the boundary between the first flat portion 725 and the second flat portion 728 continuous in the longitudinal direction A, a part of the opening edge 72 in the semi-cylindrical portion 71B of the forceps cup 71 is stepped. As shown in FIG. 9, the gap E0 formed between the second flat portion 728 and the second flat portion 628 is narrower than the gap E1 formed between the first flat portion 725 and the first flat portion 625. A part of the semi-cylindrical portion 71B of the forceps cup 71 is stepped by the stepped portion 729 formed between the first flat portion 725 and the second flat portion 728, and the rigidity of the proximal end side A2 of the forceps cup 71 is increased.

As shown in FIGS. 8 and 9, the protruding portion 626 and the protruding tooth 623 of the forceps piece 6, and the most advanced protruding tooth 724, the first distal end protruding tooth 723A and the second distal end protruding tooth 723B of the forceps piece 7 are arranged at positions where they are engaged with each other when the forceps 5 is in the closed state. When the forceps piece 6 and the forceps piece 7 are closed, the most advanced protruding tooth 724, the protruding portion 626, the first distal end protruding tooth 723A, the protruding tooth 623, and the second distal end protruding tooth 723B are arranged in this order from the distal end side A1 in the longitudinal direction A. Specifically, the protrusion 626 is arranged between the most advanced protruding tooth 724 and the first distal end protruding tooth 723A, and the protruding tooth 623 is arranged between the first distal end protruding tooth 723A and the second distal end protruding tooth 723B.

As shown in FIG. 11, the first distal end protruding tooth (first tooth) 723A of the forceps piece 7 includes a proximal end side hypotenuse (first line) 723a2 facing the protruding tooth (third tooth) 623 of the forceps piece 6 in the longitudinal direction A, and a distal end side hypotenuse 723a1 located closer to the distal end side in the longitudinal direction A than the hypotenuse 723a2. The second distal end protruding tooth 723B includes a distal end side hypotenuse 723b1 facing the protruding tooth 623 of the forceps piece 6 in the longitudinal direction A, and a proximal end side hypotenuse 723b2 located closer to the proximal end side than the distal end side hypotenuse 723b1.

As shown in FIG. 10, the protruding tooth (third tooth) 623 of the forceps piece 6 includes a hypotenuse 623a facing the first distal end protruding tooth 723A of the forceps piece 7 in the longitudinal direction A, and a hypotenuse 623b located closer to the proximal end in the longitudinal direction A than the hypotenuse 623a.

As shown in FIG. 9, the proximal end side hypotenuse 723a2 of the distal end protruding tooth 723A and the distal end side hypotenuse 623a of the protruding tooth 623 have the same length. The distal end side hypotenuse 723a1 of the first distal end protruding tooth 723A is shorter than the proximal end side hypotenuse 723a2 and the distal end side hypotenuse 623a, and the proximal end side hypotenuse 623b of the protruding tooth 623 is shorter than the distal end side hypotenuse 623a and has substantially the same length as the distal end side hypotenuse 723a1 of the distal end protruding tooth 723. Further, the distal end side hypotenuse 723b1 of the second distal end protruding tooth 723B is shorter than the distal end side hypotenuse 723a1 and the proximal end side hypotenuse 723a2 of the first distal end protruding tooth 723A. Therefore, the following relationship is satisfied: proximal end side hypotenuse 723a2>distal end side hypotenuse 723a1>distal end side hypotenuse 723b1.

When the forceps 5 is in the closed state, the protruding portion 626 and the protruding tooth 623 of the forceps piece 6 and the most advanced protruding tooth 724, the first distal end protruding tooth 723A and the second distal end protruding tooth 723B of the forceps piece 7 are engaged with each other without forming large gaps between them. As a result, the biological tissue grasped by the forceps 5 can be grasped without slipping.

As shown in FIG. 11, the second plate 73 is provided on the proximal end side A2 of the forceps cup 71, and is formed in a substantially plate shape. The plate thickness direction T4 of the second plate 73 substantially coincides with the width direction C. As shown in FIG. 6, the second plate 73 is inserted into the frame 31 and is arranged adjacent to the support plate member 38A on the right side C1 of the support plate member 38B. The first plate 63 is rotatably connected to the support plate member 38B by the pin 37b.

As shown in FIG. 11, the second plate 73 includes a first portion 73A and a second portion 73B.

The first portion 73A is formed on the distal end side A1 of the second plate 73. The first portion 73A is arranged adjacent to the first portion 63A of the forceps piece 6 on the left side C2 of the second frame piece 35 of the frame 31 shown in FIG. 6, and is located between the first portion 63A of the forceps piece 6 and the second frame piece 35.

The second portion 73B is formed on the proximal end side A2 of the first portion 73A. The second portion 63B is arranged adjacent to the support plate member 38B on the left side C2 of the second frame piece 35, and is located between the second frame piece 35 and the support plate member 38B. The thickness of the second portion 63B is thinner than that of the first portion 73A in the plate thickness direction T4, and the difference in thickness between the two portions substantially corresponds to the plate thickness of the support plate member 38B.

The second plate 73 has a first hole 731 and a second hole 732. The first hole 731 is a through-hole that penetrates the plate thickness direction T4 of the first portion 73A, and communicates with the second through-hole 35h of the second frame piece 35 shown in FIG. 6. The second hole 732 is a through-hole penetrating the second portion 73B in the plate thickness direction T4, and communicates with the second through-hole 38b of the support plate member 38B shown in FIG. 6.

As shown in FIG. 6, a pin 36 can be inserted into the first hole 731. Further, a pin 37b can be inserted into the second hole 732. The first portion 73A of the forceps piece 7 is rotatably connected to the frame 31 via the pin 36. The second portion 73B of the forceps piece 7 is rotatably connected to the support plate member 38B via the pin 37b.

As shown in FIG. 3, the forceps 5 is configured such that the forceps piece 6 and the forceps piece 7 rotate around the axis of the forceps opening/closing pin 36. The operation wire 2 for operating the forceps 5 is connected to the connecting portion 39. The connecting portion 39 is connected to the proximal end side of the forceps pieces 6 and 7 via the support plate members 38A and 38B, and by operating the operation wire 2 connected to the connecting portion 39, the forceps piece 6 and the forceps piece 7 rotate relatively in the opening/closing direction, so that the forceps 5 opens/closes.

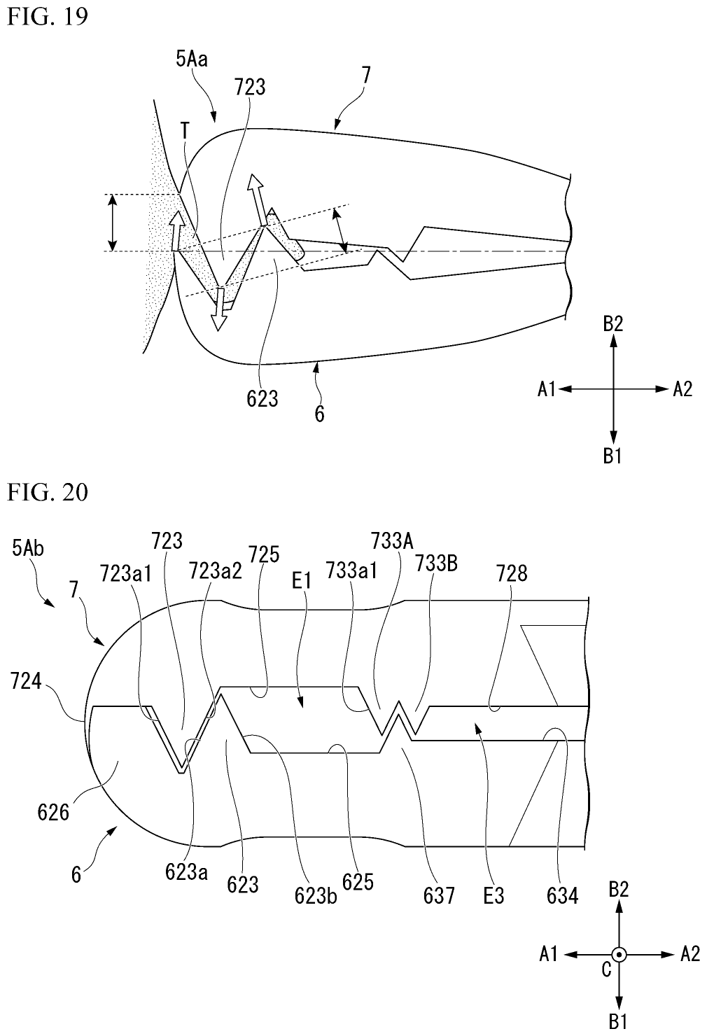

The endoscopic treatment tool 100 of the present embodiment is used together with the endoscope 200, and the forceps piece 7 and the forceps piece 6 of the forceps 5 open and close around the rotation axis R of the forceps opening/closing pin 36 intersecting the central axis O5, so that it possible to collect the target tissue T (FIG. 19). The forceps piece 6 and the forceps piece 7 of the forceps 5 are provided with a plurality of protruding teeth 623, 723A, 723B protruding toward the forceps piece 7 and the forceps piece 6, which face each other. When the forceps 5 is closed, the forceps piece 6 and the plurality of protruding teeth 623, 723A, 723B provided on the forceps pieces 7 bite into the tissue, so that the target tissue can be collected without missing.

The plurality of protruding teeth 623, 723A, 723B are formed closer to the distal end side A1 of the forceps piece 6 and the forceps piece 7 than a half length of each forceps cup 61, 71 in the longitudinal direction A. Therefore, the gripping force of the distal end side A1 of the forceps 5 is increased, and the tissue is torn at the distal end side A1 so that less tissue will be missed. In the present embodiment, the length of the forceps cups 61 and 71 in the longitudinal direction A is about 2 mm, but the length is not limited to this.

Further, by providing the most advanced protruding tooth 724 at the distal end of the forceps 5, the force to bite into the target tissue T becomes large, and the target tissue T can be grasped and collected with a small force.

The forceps 5 in this embodiment is not limited to the above configuration, and may have other structures.

Hereinafter, a modification of the forceps in the first embodiment will be described. In the following description, the same reference numerals will be given to the configurations common to those already described, and duplicate description will be omitted. In each of the following drawings, in order to make each component easy to see, the scale of the dimensions may be different depending on the component.

### Modification Example 1-0

In the first embodiment, when the forceps piece 6 and the forceps piece 7 are closed, the distal end side A1 of both is engaged without forming a large gap, but the present disclosure is not limited to this. Further, in the first embodiment, a plurality of protruding teeth and a plurality of protruding portions are provided only on the distal end side A1 of the forceps 5, but the present disclosure is not limited to this.

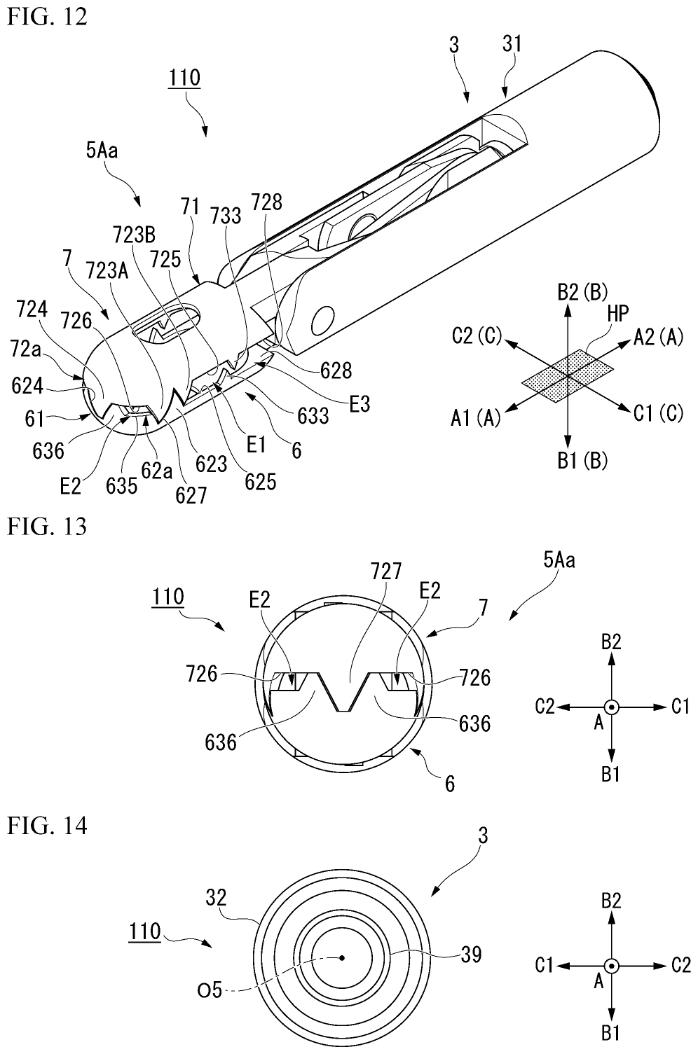

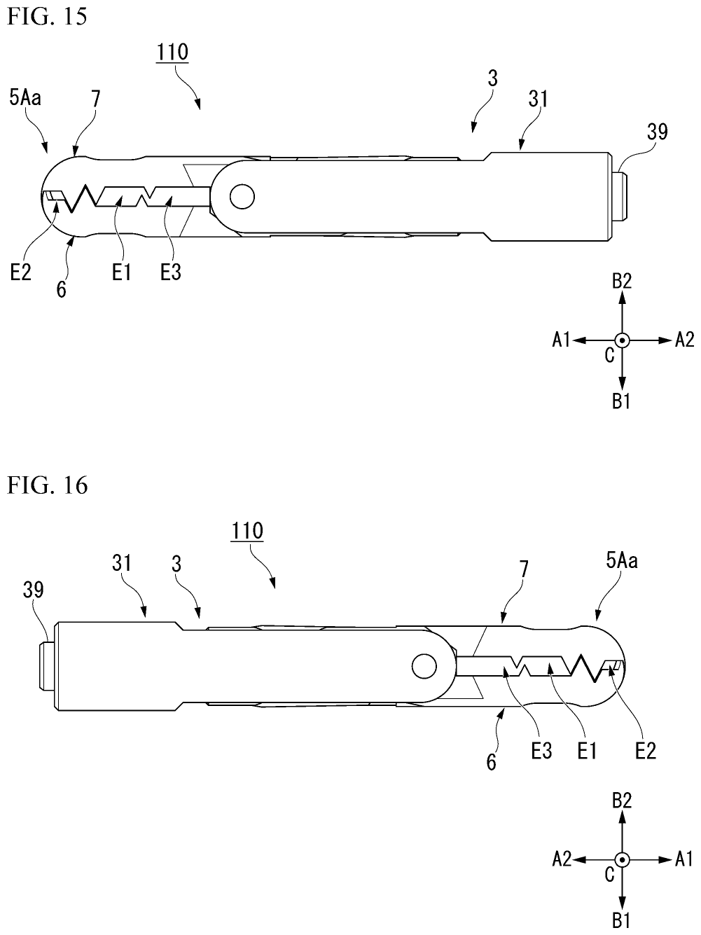

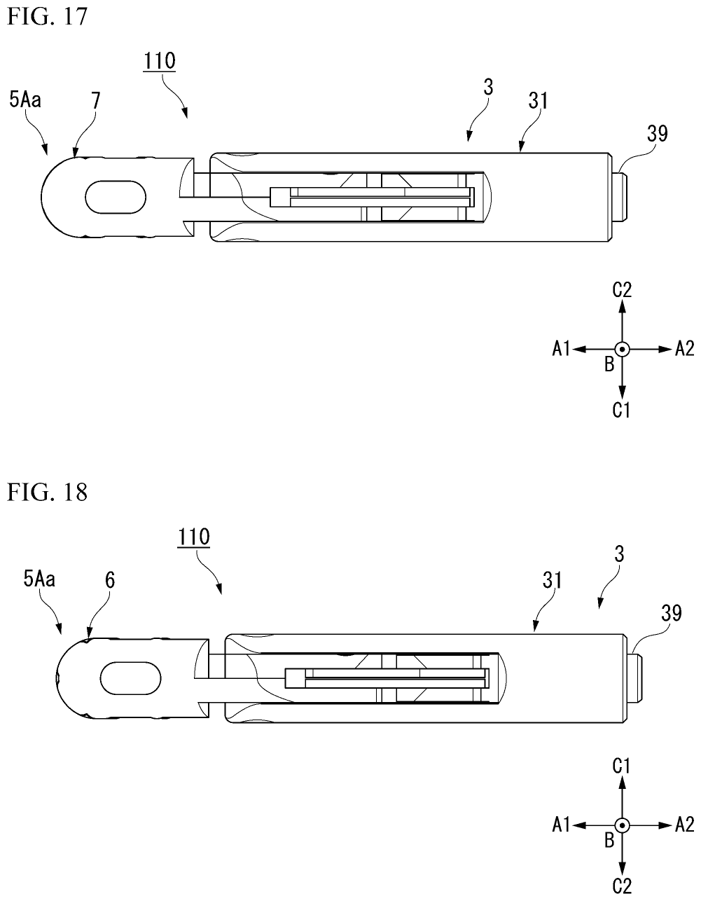

FIG. 12 is a perspective view of the distal end of a forceps 5Aa of the treatment portion 110 in the modification 1-0. FIG. 13 is a front view of the forceps 5Aa of the treatment portion 110 in the modification 1-0. FIG. 14 is a rear view of the forceps 5Aa of the treatment portion 110 in the modification 1-0. FIG. 15 is a right side view of the forceps 5Aa of the treatment portion 110 in the modification 1-0. FIG. 16 is a left side view of the forceps 5Aa of the treatment portion 110 in the modification 1-0. FIG. 17 is a plan view of the forceps 5Aa of the treatment portion 110 in the modification 1-0. FIG. 18 is a bottom view of the forceps 5Aa of the treatment portion 110 in the modification 1-0. FIG. 19 is a schematic view of the forceps 5Aa in the modification 1-0.

As the forceps 5Aa of the modification 1-0 shown in FIGS. 12 to 18, a configuration may be adopted in which a gap E2 is formed in the distal end side A1 when the forceps 5Aa is closed, and a configuration may be adopted in which a plurality of protruding teeth 633 and 733 are also provided on the proximal end side A2 of the forceps 5Aa.

As shown in FIG. 12, the forceps 5Aa of the modification 1-0 includes a pair of proximal protruding teeth 633 and 733 protruding toward the forceps pieces 6 and 7A facing each other on the proximal end side A2 of the forceps cups 61 and 71. These plurality of proximal end protruding teeth 633 and 733 are formed closer to the proximal end side A2 than a half length of each forceps cups 61 and 71 in the longitudinal direction A. The proximal end protruding tooth (second tooth) 733 of the forceps piece 7 is provided on the proximal end side A2 of the proximal end protruding tooth 633 of the forceps piece 6. On the other hand, the proximal end protruding tooth (second tooth) 733 of the forceps piece 7 may be provided on the distal end side A1 of the proximal end protruding tooth 633 of the forceps piece 6.

The proximal end protruding teeth 633 and 733 have a substantially triangular shape when viewed from the width direction C. The proximal end protruding teeth 633 and 733 are smaller than the protruding tooth 623, the first distal end protruding tooth 723A, and the second distal end protruding tooth 723B provided on the distal end side, and have substantially the same size as each other.

The opening edge 62 of the forceps cup 61 includes a distal end groove portion 624, a pair of protruding teeth 636, a pair of recesses 635, a pair of side groove portions 627, a pair of protruding teeth 623, a pair of first flat portions 625, a pair of proximal end protruding teeth 633, and a pair of second flat portions 628.

An opening edge 72a of the forceps cup 71 includes a most advanced protruding tooth 724, a pair of recesses 726, a pair of first distal end protruding teeth 723A, a pair of second distal end protruding teeth 723B, a pair of first flat portions 725, a pair of proximal end protruding teeth 733, and a pair of second flat portions 728.

When the forceps 5Aa is closed, the pair of protruding teeth 636 of the forceps piece 6 are housed in the pair of recesses 726 on both sides of the most advanced protruding tooth 724 of the forceps piece 7. The portion of the recess 726 of the forceps piece 7 other than the region in which the protruding tooth 636 is housed faces the recess 635 of the forceps piece 6 in the vertical direction B. A gap E2 is formed between a part of the pair of recesses 726 of the forceps piece 7 and the pair of recesses 635 of the forceps piece 6. Specifically, the gap E2 is a gap formed by the protruding tooth 636 and the recess 635 of the forceps piece 6 and the recess 726 and the first distal end protruding tooth 723A of the forceps piece 7 when the forceps 5Aa is closed.

The size of the gap E2 is not limited to the size shown in the figure, and can be changed as appropriate.

In this example, a pair of gaps E2 are formed in the proximal end side A2 when the forceps 5Aa is closed, so that by letting the tissue enter into the gap E2, the thickness of the tissue sandwiched by the most advanced protruding tooth 724 of the distal end side A1 and the pair of protruding teeth 636 can be reduced, and the gripping force of the proximal end side A2 can be increased.

Further, when the forceps 5Aa is closed, the most advanced protruding tooth 724 of the forceps piece 7 and the pair of protruding teeth 636 of the forceps pieces 6 are engaged with each other without forming a large gap between them, and the first distal end protruding tooth 723A and the second distal end protruding tooth 723B of the forceps piece 7 and the protruding tooth 623 of the forceps piece 6 are engaged with each other without forming a large gap between them. On the other hand, the pair of proximal end protruding teeth 733 provided on the proximal end side A2 of the forceps piece 7 and the pair of proximal end protruding teeth 633 provided on the proximal end side A2 of the forceps piece 6 are not engaged with each other, and are arranged apart from each other in the longitudinal direction A.

Further, when the forceps 5Aa is closed, a pair of second flat portions 628 provided on the proximal end side A2 of the forceps piece 6 and a pair of second flat portions 728 provided on the proximal end side A2 of the forceps piece 7 face each other in the vertical direction, and a gap E3 is formed between the second flat portion 628 and the second flat portion 728.

In this example, when the forceps 5Aa is closed, while the target tissue grasped by the plurality of protruding teeth 724, 636, 723A, 623, 723B provided on the distal end side A1 is supplementally grasped by the proximal protruding teeth 633, 733, the grasped tissue can be released to the outside from the gaps E1 and E3 formed between the forceps pieces 6 and 7, so that a larger tissue can be collected.

In the first embodiment and the modification 1-0 described above, regarding the lengths of the two hypotenuses 723a1 and 723a2 of the first distal end protruding tooth 723A and the two hypotenuses 723b1 of the second distal end protruding tooth 723B in the forceps piece 7, the proximal end side hypotenuse 723a2 of the first distal end protruding tooth 723A is the longest, and the distal end side hypotenuse 723a1 of the first distal end protruding tooth 723A and the distal end side hypotenuse 723b1 of the second distal end protruding tooth 723B are shortened in this order.

Therefore, when the forceps 5Aa is closed, the protruding tooth 723 of the forceps piece 7 are engaged with the protruding tooth 623 of the forceps piece 6, so that a part of the tissue T can be sandwiched by being bent up and down as shown in FIG. 19, and the bending width of the tissue T by each of the protruding teeth 723 and 623 can be widened. As a result, when the target tissue T is grasped by the forceps 5, the tissue T is deeply bitten into by the proximal end side A2 of the forceps 5Aa, so that the tissue T is reliably held.

### (Modification 1-1)

FIG. 20 is a right side view showing a forceps 5Ab of the modification 1-1.

The forceps 5Ab shown in FIG. 20 includes a plurality of protruding teeth that are engaged with each other on the proximal end side of the forceps piece (second gripping piece) 6 and the forceps piece (first gripping piece) 7. Specifically, the forceps piece 7 includes a most advanced protruding tooth 724 and a pair of distal end protruding teeth (first tooth) 723 provided closer to the distal end side A1 than a half length of the forceps cup 71 in the longitudinal direction A, and a pair of first proximal end protruding teeth (second tooth) 733A and a pair of second proximal end protruding teeth 733B provided closer to the proximal end side A2 than a half length of the forceps cup 71 in the longitudinal direction A. The first proximal end protruding tooth (second tooth) 733A is arranged on the proximal end side A2 of the first distal protruding tooth (first tooth) 723 via the first flat portion 725.

The forceps piece 6 includes a pair of protruding portions 626 and a pair of protruding teeth (third tooth) 623 provided closer to the distal end side A1 than a half length of the forceps cup 61 in the longitudinal direction A, and a pair of proximal end protruding teeth 637 provided closer to the proximal end side A2 than a half length of the forceps cup 61 in the longitudinal direction A. The proximal end protruding tooth 637 is arranged on the proximal end side A2 of the protruding tooth 623 via the first flat portion 625.

When the forceps 5Ab is in the closed state, the distal end protruding tooth 723 of the forceps piece 7 and the protruding tooth 623 of the forceps piece 6 are aligned in the longitudinal direction A and are engaged with each other without forming a large gap. At this time, the distal end protruding tooth 723 is arranged closer to the distal end side A1 than the protruding tooth 623. Further, the proximal end protruding tooth 637 is arranged between the proximal end protruding tooth 733A and the second proximal end protruding tooth 733B.

The distal end protruding tooth 723 includes a proximal end side hypotenuse (first side) 723a2 facing the protruding tooth 623 in the longitudinal direction A, and a distal end side hypotenuse (second side) 723a1 located closer to the distal end side A1 in the longitudinal direction A than the proximal end side hypotenuse 723a2.

The protruding tooth (third tooth) 623 includes a distal end side hypotenuse (third side) 623a facing the distal end protruding tooth 723 in the longitudinal direction A, and a proximal end side hypotenuse (fourth side) 623b located closer to the proximal end side A2 in the longitudinal direction A than the distal end side hypotenuse 623a.

The proximal end side hypotenuse 723a2 of the distal end protruding tooth 723 and the distal end side hypotenuse 623a of the protruding tooth 623 have the same length as each other. The distal end side hypotenuse 723a1 of the distal end protruding tooth 723 is shorter than the proximal end side hypotenuse 723a2 and the distal end side hypotenuse 623a of the protruding tooth 623. Further, the proximal end side hypotenuse 623b of the protruding tooth 623 has substantially the same length as the distal end side hypotenuse 723a1 of the distal end protruding tooth 723.

When the forceps 5Ab is in the closed state, the first proximal end protruding tooth (second tooth) 733A and the second proximal end protruding tooth 733B of the forceps piece 7 and the proximal end protruding tooth 637 of the forceps piece 6 are aligned in the longitudinal direction A and are engaged with each other without forming a large gap. When the forceps 5Ab is in the closed state, the proximal end protruding tooth 637 of the forceps piece 6 is arranged between the first proximal end protruding tooth (second tooth) 733A and the second proximal end protruding tooth 733B.

The distal hypotenuse 733a1 of the proximal end protruding tooth 733A is shorter than the proximal hypotenuse 723a2 and the distal hypotenuse 723a1 of the distal protruding tooth 723. Regarding the size relationship between the distal end side hypotenuse 733a1 of the first proximal end protruding tooth 733A and the proximal end side hypotenuse 723a2 and the distal end side hypotenuse 723a1 of the distal end protruding tooth 723, the following relationship is satisfied: the proximal end side hypotenuse 723a2 of the distal end protruding tooth 723>the distal end side hypotenuse 723a1>the distal end side hypotenuse 733a1 of the first proximal end protruding tooth

In the forceps 5Ab of this example, the most advanced protruding tooth 724, the distal end protruding tooth 723, and the protruding tooth 623 are provided in the distal end side A1, and the protruding teeth 733A and 733B, which are smaller than the protruding teeth 724, 723, 623, and the protruding tooth 637 is provided in the proximal end side A2. Since these protruding teeth 733A, 733B, and 637 are arranged closer to the proximal end side A2 than the protruding teeth 724, 723, 623, the gripping force on the distal end side A1 of the forceps 5Ab can be increased, and a part of the target tissue T grasped on the distal end side A1 of the forceps 5Ab can also be grasped on the proximal end side A2 of the forceps 5Ab. By grasping the target tissue T at two points before and after the forceps 5Ab, it is possible to collect the target tissue T without missing. Further, by providing the protruding teeth 733A, 733B, 637 smaller than the protruding teeth 724, 723, 623 provided on the distal end side A1 on the proximal end side A2, it is possible to increase the gripping force of the distal end side A1 without hindering the engagement of the distal end side A1.

In this embodiment, the forceps piece 7 has small protruding teeth 733A and 733B, but the forceps piece 6 may have two small protruding teeth.

### (Modification 1-2)

The plurality of protruding teeth shown in the previous embodiment have a substantially constant thickness in the longitudinal direction A, but the thickness is not limited to this. For example, in at least one protruding tooth among the plurality of protruding teeth, the thickness of the protruding tooth may change in the longitudinal direction A.

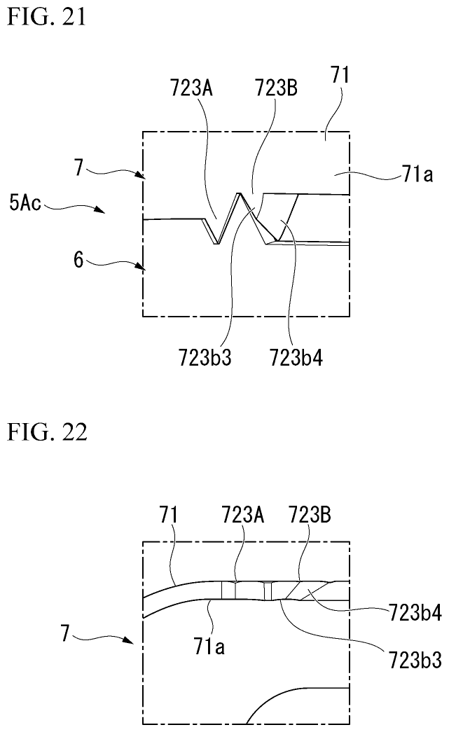

FIG. 21 is a view showing a part of a forceps 5Ac of the modification 1-2, and is a view seen from the inside of the forceps pieces 6 and 7. FIG. 22 is a bottom view showing the forceps piece 7 of the modification 1-2.

In the forceps 5Ac shown in FIGS. 21 and 22, for example, the thickness of the second distal end protruding tooth 723B of the forceps piece 7 is formed to change in the longitudinal direction A. Specifically, in the plan view shown in FIG. 22, the second distal end protruding tooth 723B includes an inner surface 723b3 continuous with an inner peripheral surface 71a of the forceps cup 71, and an inner inclined surface 723b4 inclined at a predetermined angle with respect to the central axis O5. The inner inclined surface 723b4 is inclined in a direction away from the central axis O5 (outward in the radial direction) from the distal end side A1 to the proximal end side A2. That is, the wall thickness of the second distal end protruding tooth 723B gradually decreases from a predetermined position on the distal end side A1 toward the proximal end side A2, and the proximal end side A2 has a sharp edge shape.

According to the forceps 5Ac in this example, since the proximal end side A2 of the second distal end protruding tooth 723B is sharpened, a cut can be made in the grasped target tissue T, and the target tissue T is easily torn. Therefore, the target tissue T can be easily collected with a small force.

### (Modification 1-3)

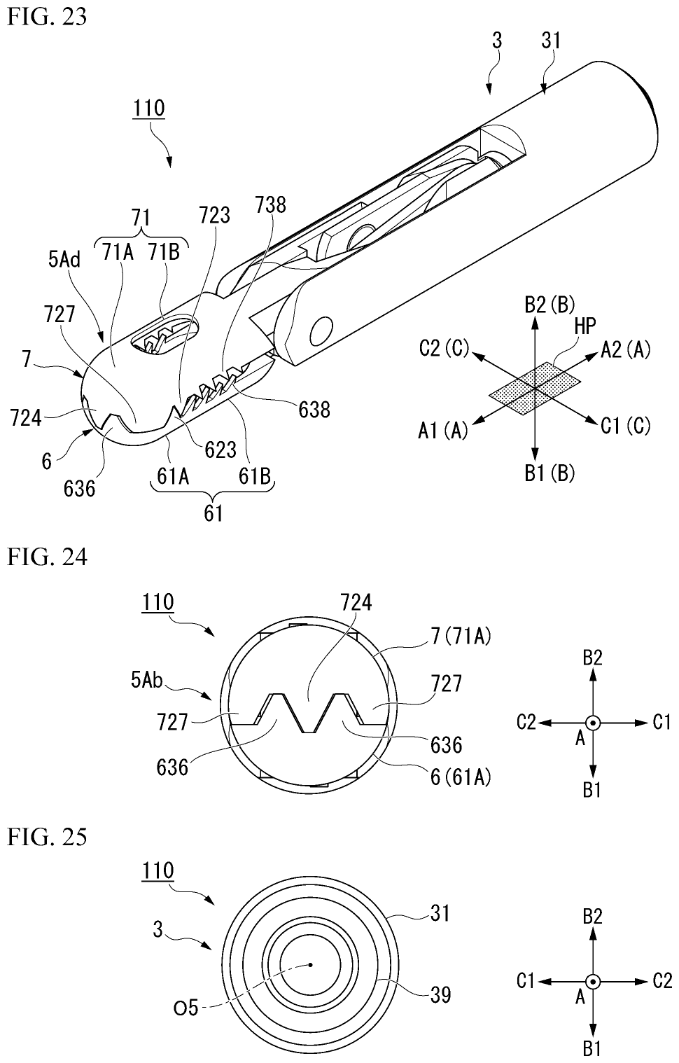

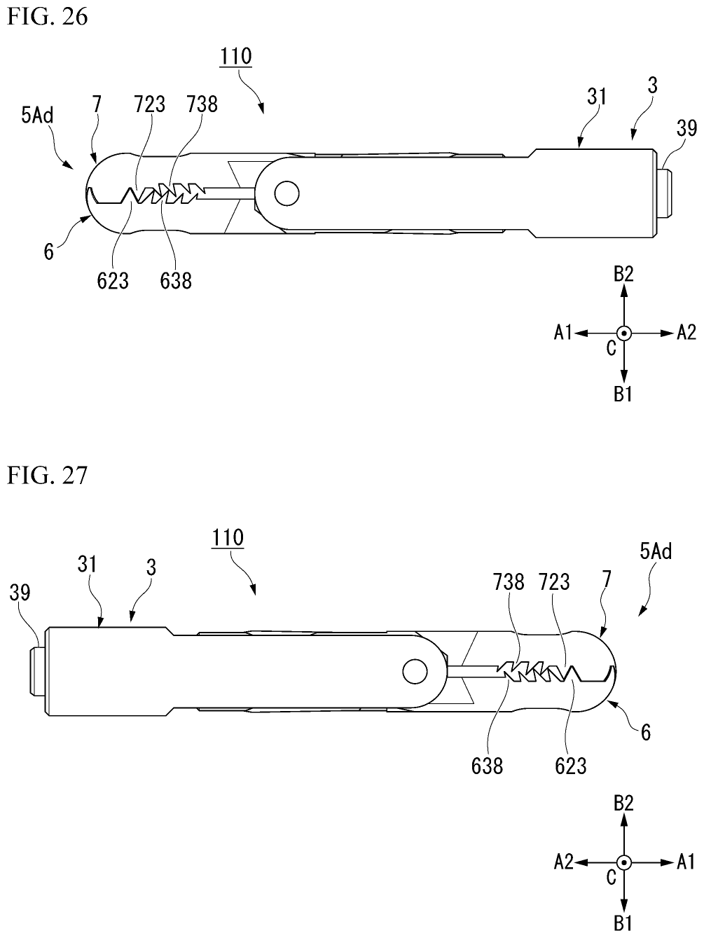

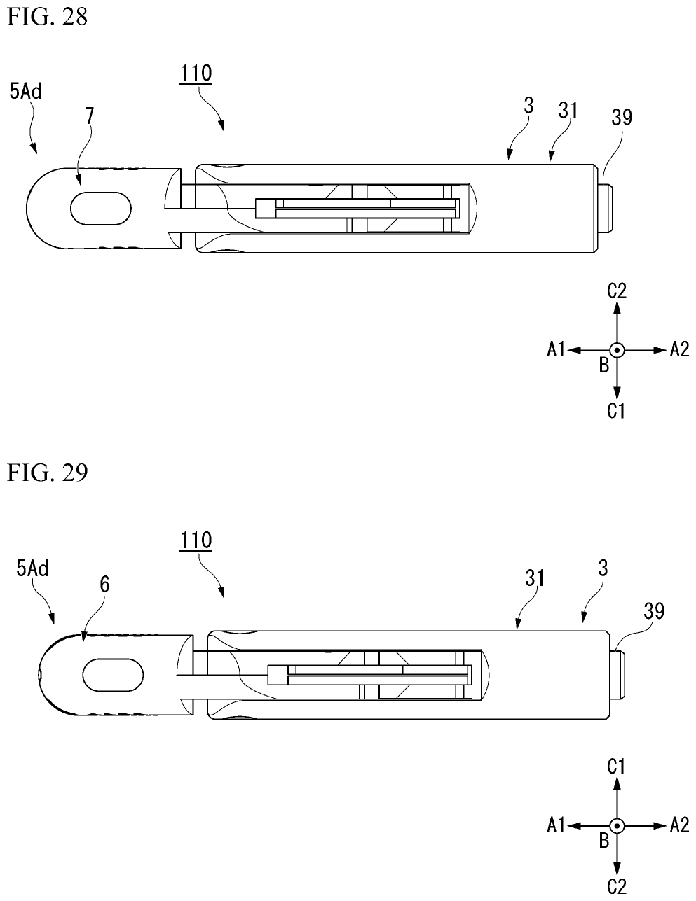

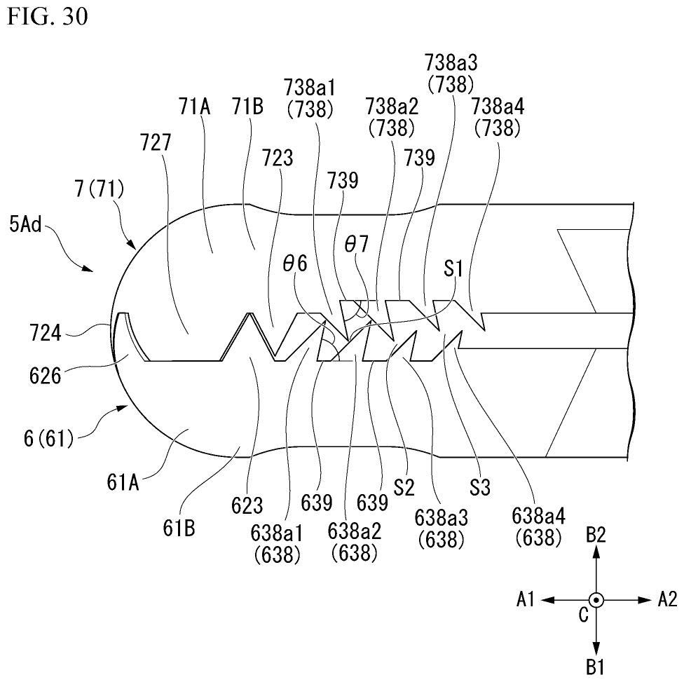

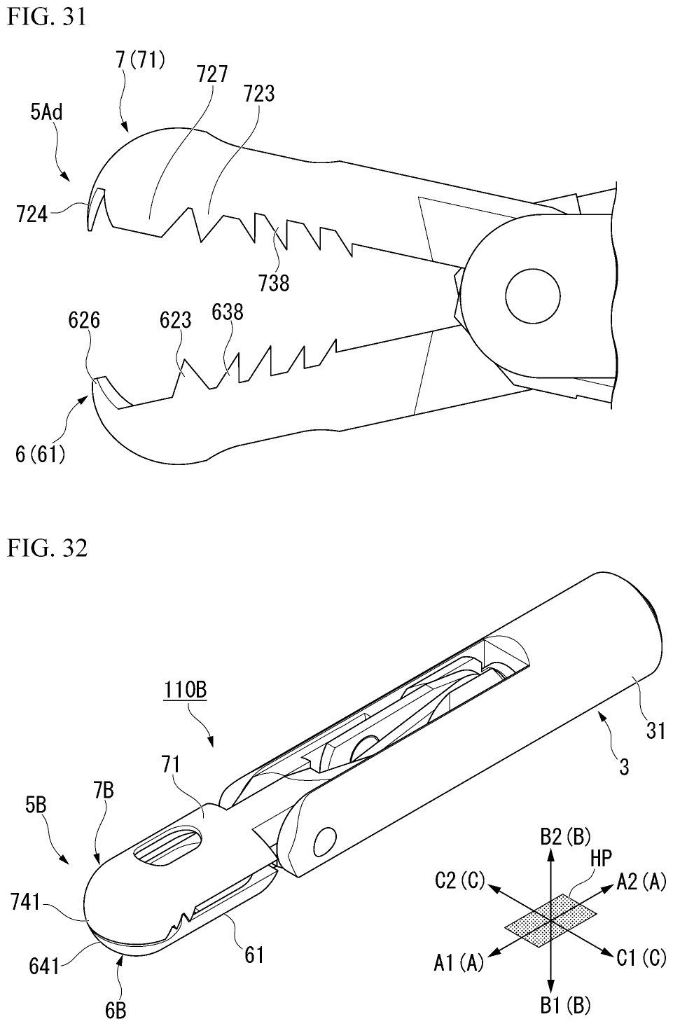

FIG. 23 is a perspective view showing a forceps 5Ad of the modification 1-3 in the treatment portion 110. FIG. 24 is a front view showing the forceps 5Ad of the modification 1-3 in the treatment portion 110. FIG. 25 is a rear view showing the forceps 5Ad of the modification 1-3 in the treatment portion 110. FIG. 26 is a right side view showing the forceps 5Ad of the modification 1-3 in the treatment portion 110. FIG. 27 is a left side view showing the forceps 5Ad of the modification 1-3 in the treatment portion 110. FIG. 28 is a plan view showing the forceps 5Ad of the modification 1-3 in the treatment portion 110. FIG. 29 is a bottom view showing the forceps 5Ad of the modification 1-3 in the treatment portion 110. FIG. 30 is a partially enlarged view showing the closed state of the forceps 5Ad of the modification 1-3. FIG. 31 is a partially enlarged view showing the open state of the forceps 5Ad of the modification 1-3.

The forceps 5Ad of this example shown in FIGS. 23 to 31 includes a forceps piece 7 having a most advanced protruding tooth 724, a pair of protruding portions 727, and a pair of protruding teeth 723 on the distal end portion 71A of the forceps cup 71, and a forceps piece 6 having a pair of protruding teeth 636 and a pair of protruding teeth 623 at the distal end portion 61A of the forceps cup 61.

The forceps 5Ad of this example shown in FIGS. 23 to 31 includes a plurality of return teeth 638 and 738, at substantially the center of the length A of each forceps cup 61 and 71 in the forceps pieces 6 and 7 in the longitudinal direction. The plurality of return teeth 638 and 738 are provided on the semi-cylindrical portions 61B and 71B on the proximal end side A2 of the protruding teeth 623 and 723 of the forceps pieces 6 and 7.

In this example, as shown in FIGS. 23 and 26, each forceps piece 6 and 7 is provided with four return teeth 638 and 738, but the number of the return teeth is not limited to this. For example, different numbers of return teeth 638 and 738 may be provided between the forceps pieces 6 and 7.

A flat surface 639 exists between the plurality of return teeth 638. A flat surface 739 exists between the plurality of return teeth 738.

As shown in FIG. 30, the return teeth 638 and 738 are all tilted at a predetermined angle with respect to the central axis O5 from the distal end side A1 to the proximal end side A2. The inclination angle θ6 of the return tooth 638 is an angle formed by the hypotenuse of the return tooth 638 on the proximal end side A2 and the flat surface 639. The inclination angle θ7 of the return tooth 738 is an angle formed by the hypotenuse of the return tooth 738 on the proximal end side A2 and the flat surface 739. The inclination angles θ6 and θ7 of the return teeth 638 and 738 are acute, 90° or less.

The plurality of return teeth 638 may be tilted at an angle θ6 equal to each other, or all or some of the return teeth 638 may be tilted at different angles θ6. Similarly, the plurality of return teeth 738 may be tilted at the same angle θ7, or all or some of the return teeth 738 may be tilted at different angles θ7.

The protruding heights of the plurality of return teeth 638 are all lower than the protruding heights of the protruding tooth 623, and the protruding heights of the return teeth 638 located at the proximal end side A2 are lower.

The protruding heights of the plurality of return teeth 738 are all lower than the protruding heights of the distal end protruding teeth 723, and the protruding heights of the return teeth 738 located on the proximal end side A2 are lower.

The protruding heights of the plurality of return teeth 638 and the plurality of return teeth 738 are not limited to the heights shown. The protrusion heights of the plurality of return teeth 638 and the plurality of return teeth 738 may be the same, or the protrusion heights of some of the return teeth 638 and 738 may be different.

As shown in FIG. 30, when the forceps 5Ad is closed, the plurality of return teeth 638a1 to 638a4 and the plurality of return teeth 738a1 to 738a4 are alternately arranged in the longitudinal direction A. At this time, the return teeth 638a1 to 638a4 are located on the distal end side A1 rather than the return teeth 738a1 to 738a4. The plurality of return teeth 638a1 to 638a4 and 738a1 to 738a4 are all inclined toward the proximal end side A2. Therefore, the tooth tips of the three return teeth 738a1 to 738a3 from the distal end side A1 face the hypotenuse on the distal end side of the adjacent return teeth 638a1 to 638a3 in the longitudinal direction A. The return tooth 638 of the forceps piece 6 does not exist on the proximal end side A2 more than the return tooth 738a4 located on the most proximal end side A2. Further, the return tooth 638a1 located on the most distal end side A1 of the forceps piece 6 faces the hypotenuse of the distal end side A1 of the return tooth 738a1 located on the most distal end side A1 of the forceps piece 7.

The distance between the distal end of each return tooth 638 and the hypotenuse of the distal end side A1 of the return tooth 738 facing the tooth tip is different at the arrangement positions of the plurality of return teeth 638 and 738.

Of the plurality of return teeth 638 and the plurality of return teeth 738, the distance between the return tooth 738a1 located at the most distal end side A1 and the return tooth 638a2 facing thereto is set as a distance S1, the distance between the return tooth 738a2 and the return tooth 638a3 facing thereto is set as a distance S2, and the distance between the return tooth 738a3 and the return tooth 638a4 facing thereto is set as a distance S3. Then, the relationship between the sizes of the distances S1 to S3 is S1<S2<S3, and the distance between the opposing return teeth 638 and 738 becomes wider toward the proximal end side A2.

As in the forceps 5Ad of this example, each forceps piece 6 and 7 may be provided with a plurality of return teeth 638 and 738. Since the surface of the living tissue is in a wet state, if the target tissue T is pulled with forceps when collecting the target tissue T, the target tissue T may slip to the distal end side A1 of the forceps, thereby making it difficult to collect the target tissue T. Therefore, as in this example, by providing a plurality of return teeth 638 and 738 near the center of the forceps pieces 6 and 7 in the length direction, when the grasped target tissue T slides out to the distal end side A1, the return teeth 638 and 738 bite into it, so that the target tissue T can be fixed near the center of the forceps pieces 6 and 7 in the length direction, and slipping to the distal end side A1 can be suppressed.

Further, since all the return teeth 638 and 738 are inclined to the proximal end side A2 and the respective inclination angles θ6 and θ7 are acute angles, the tooth tips of the return teeth 638 and 738 are sharpened, making it easier to bite into the target tissue T from the distal end side A1. As a result, the effect of suppressing the slip of the target tissue T to the distal end side A1 is high.

Further, by reducing the size of the return teeth 638 and 738 toward the proximal end side A2, the facing distance between the return teeth 638 and 738 increases toward the proximal end side A2. With such a shape, the gripping force of the distal end side A1 of the forceps 5 can be increased.

Further, as shown in FIG. 31, even when the forceps 5Ad is slightly opened, the tooth tips of the return teeth 638 and 738 extend in the direction of being caught in the grasped target tissue T, so that the number of the tooth tips of the return teeth 638 and 738 biting into the target tissue T increases as the forceps pieces 6 and 7 are closed. As a result, the target tissue T can be grasped without missing.

### Second Embodiment

An endoscopic treatment tool 100B (FIG. 1) according to the second embodiment of the present disclosure will be described with reference to the drawings.

In the following description, the same reference numerals will be given to the configurations common to those already described, and duplicate description will be omitted.

[Endoscopic treatment tool 100B]

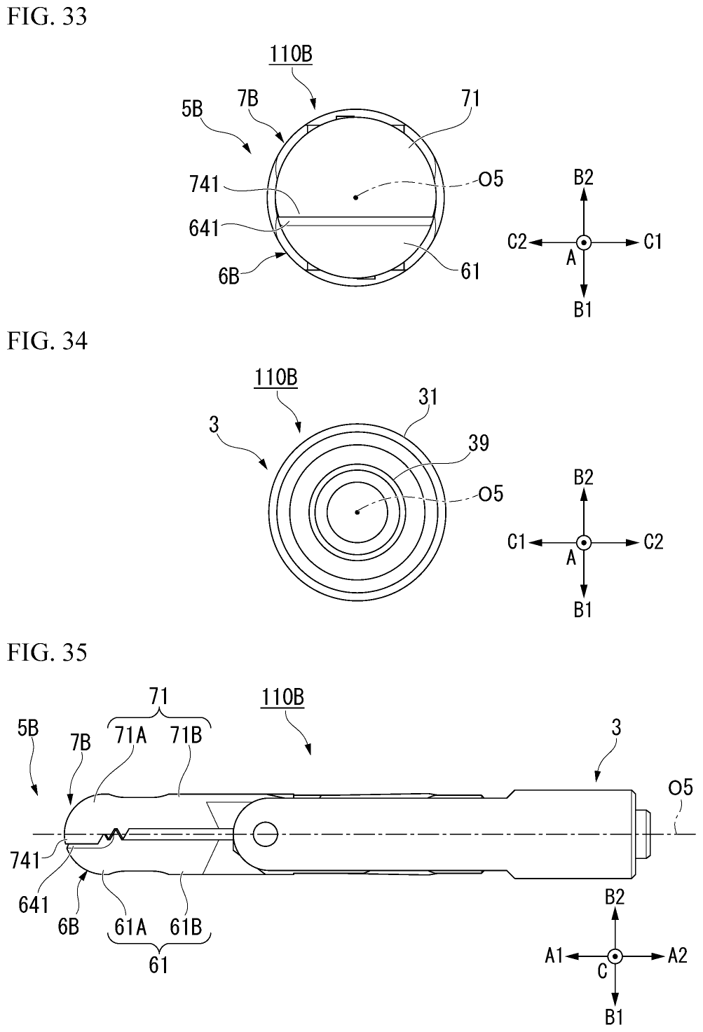

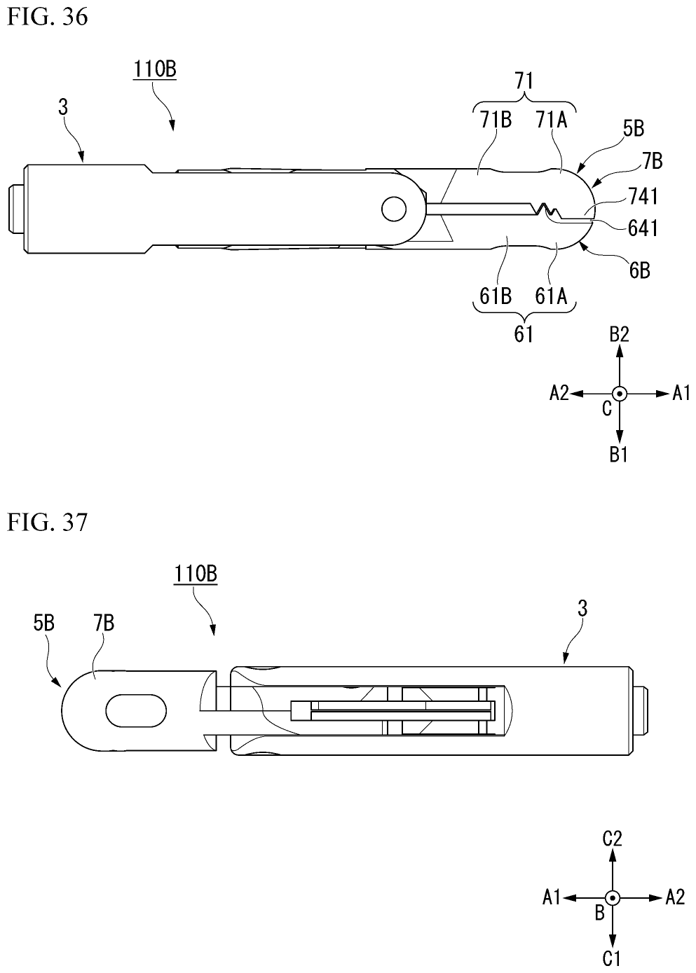

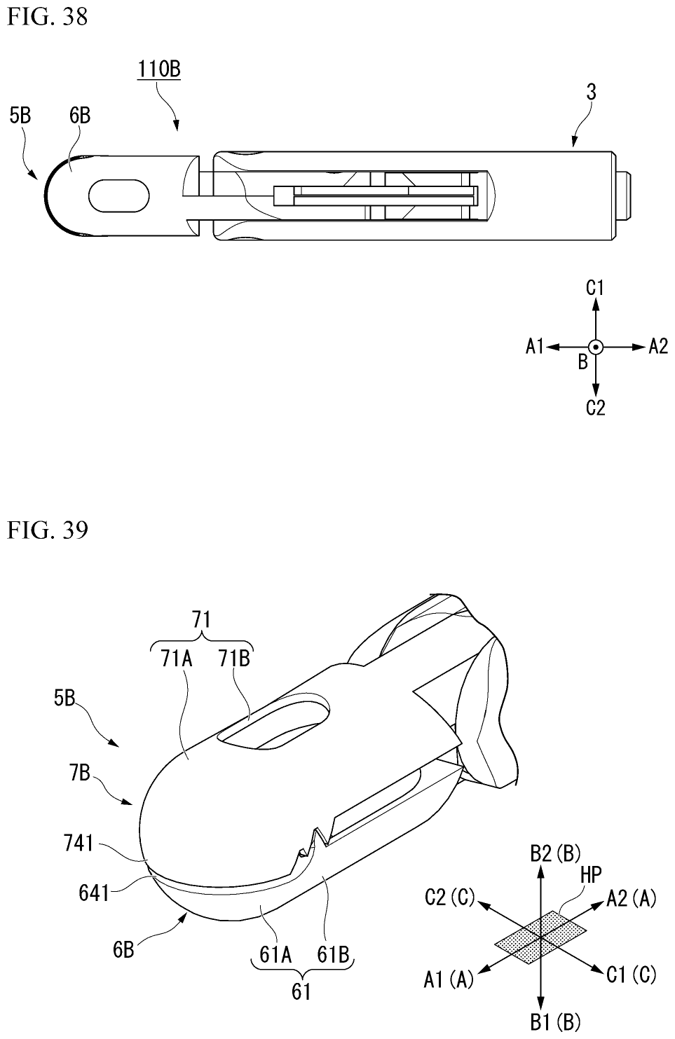

FIG. 32 is a perspective view showing forceps 5B of a treatment portion 110B according to the second embodiment. FIG. 33 is a front view showing the forceps 5B of the treatment portion 110B according to the second embodiment. FIG. 34 is a rear view showing the forceps 5B of the treatment portion 110B according to the second embodiment. FIG. 35 is a right side view showing the forceps 5B of the treatment portion 110B according to the second embodiment. FIG. 36 is a left side view showing the forceps 5B of the treatment portion 110B according to the second embodiment. FIG. 37 is a plan view showing the forceps 5B of the treatment portion 110B according to the second embodiment. FIG. 38 is a bottom view showing the forceps 5B of the treatment portion 110B according to the second embodiment. FIG. 39 is a partially enlarged perspective view of the forceps 5B according to the second embodiment.

The endoscopic treatment tool 100B of the second embodiment shown in FIG. 1 (simply also referred to as a treatment tool 100B) is used as an endoscopic treatment system together with the endoscope 200 like the endoscopic treatment tool 100 of the first embodiment. The treatment tool 100B includes a sheath 1, an operation wire 2, a support member 3, forceps 5B, and an operating portion 8.

The forceps 5B of the present embodiment shown in FIGS. 32 to 38 is a member for collecting living tissue. The forceps 5B is made of a metal material such as stainless steel, and includes a forceps piece (first gripping piece) 7B and a forceps piece (second gripping piece) 6B.

FIG. 39 is an enlarged view showing forceps 5B of the endoscopic treatment tool 100B according to the second embodiment.

As shown in FIG. 39, the forceps piece (first gripping piece) 7B has a first scissors tooth 741 on the most distal end side A1. The first scissors tooth 741 is formed over substantially the entire arcuate opening edge of the distal end portion 71A of the forceps cup 71. Both ends of the proximal end side A2 of the first scissors tooth 741 extend to the vicinity of the boundary between the distal end portion 71A of the forceps cup 71 and the semi-cylindrical portion 71B. The thickness of the first scissors tooth 741 gradually decreases toward the facing forceps piece (second gripping piece) 6B, and the tooth tip has a sharp shape.

The forceps piece 6B has a second scissors tooth 641 on the most distal end side A1. The second scissors tooth 641 is formed over substantially the entire arcuate opening edge of the distal end portion 61A of the forceps cup 61. Both ends of the proximal end side A2 of the second scissors tooth 641 extend to the vicinity of the boundary between the distal end portion 61A of the forceps cup 61 and the semi-cylindrical portion 61B. The thickness of the second scissors tooth 641 gradually decreases toward the facing forceps piece 7B, and the cutting edge has a sharp shape.

In the present embodiment, the first scissors tooth 741 and the second scissors tooth 641 are formed over substantially the entire circumference of the open ends at the distal end portions 71A and 61A of the forceps pieces 7B and 6B, but the present disclosure is not limited to this configuration. The first scissors tooth 741 and the second scissors tooth 641 may be formed only on a part of the distal end portions 71A and 61A of the forceps pieces 7B and 6B.

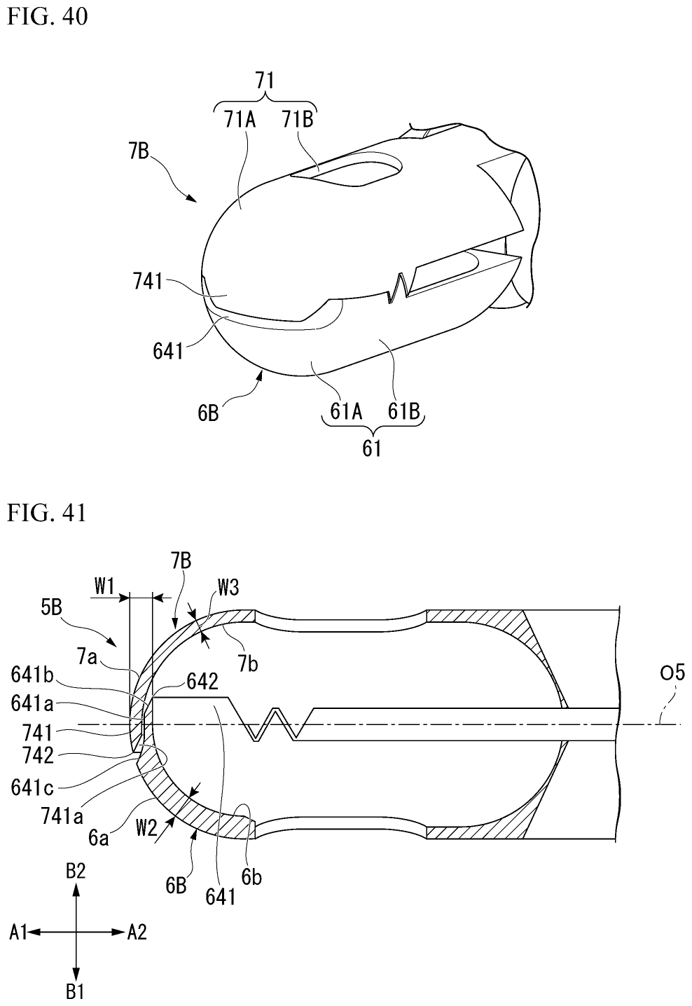

FIG. 40 is a perspective view showing another embodiment of the forceps pieces 7B and 6B.

For example, as shown in FIG. 40, the first scissors tooth 741 and the second scissors tooth 641 may be partially formed only in a predetermined range including the cutting edge of the distal end portions 71A and 61A. In this configuration, both ends of the proximal end side A2 of the first scissors tooth 741 and the second scissors tooth 641 are located closer to the distal end side A1 than the boundary between the distal end portions 71A and 61A of the forceps cups 71 and 61 and the semi-cylindrical portions 71B and 61B.

FIG. 41 is a partial cross-sectional view showing forceps 5B of the endoscopic treatment tool 100B according to the second embodiment.

As shown in FIG. 41, when the forceps 5B is closed, the first scissors tooth 741 of the forceps piece 7B are arranged closer to the distal end side A1 in the longitudinal direction A than the second scissors tooth 641 of the forceps piece 6B. The first scissors tooth 741 and the second scissors tooth 641 overlap each other on the extension of the central axis O5. The outer peripheral surface 7a of the forceps piece 7B and the outer peripheral surface 6a of the forceps piece 6B form an arc shape having substantially the same center existing on the central axis O5, and the outer peripheral surface 6a exists on an extension line of the outer peripheral surface 7a. The second scissors tooth 641 has a recess 641c formed by the first tooth surface 641a curved inward in the longitudinal direction A (proximal end side A2) on the distal end side A1, and the first scissors tooth 741 is arranged when the forceps 5B is closed.

The total thickness W1 of the first scissors tooth 741 and the second scissors tooth 641 overlapping in the longitudinal direction A when the forceps 5B is closed is substantially equal to the thickness W2 of a part of the distal end portion 61A of the forceps piece 6B excluding the second scissors tooth 641. The maximum thickness W3 of a part of the distal end portion 71A of the forceps piece 7B excluding the first scissors tooth 741 is thinner than the maximum thickness W2 of a part of the distal end portion 61A of the forceps piece 6.

As described above, in the present embodiment, when the forceps 5B is closed, the first scissors tooth 741 and the second scissors tooth 641 overlap on the central axis O5, so that the protruding heights of the first scissors tooth 741 and the second scissors tooth 641 can be increased without changing the length of the forceps 5B in the vertical direction B. As a result, in the first scissors tooth 741 and the second scissors tooth 641, the cut becomes deeper with respect to the grasped tissue T.

The first scissors tooth 741 has a tooth surface 741a on the inner peripheral surface 7b side facing the second scissors tooth 641 when the forceps 5B is in a closed state. The tooth surface 741a is inclined toward the distal end side A1 as the forceps piece 7B closes (lower side B1), and is connected to the outer peripheral surface 7a at the lowermost end. The tissue T can be cut by the cutting edge 742 formed at the intersecting ridge line between the outer peripheral surface 7a and the tooth surface 741a.

The second scissors tooth 641 has a first tooth surface 641a and a second tooth surface 641b on the outer surface side facing the first scissors tooth 741 when the forceps 5B is in a closed state. The first tooth surface 641a curves toward the proximal end side A2 (inner peripheral surface 6b side of the forceps piece 6B) as the forceps piece 6B closes (upper side B2). The second tooth surface 641b is further inclined toward the proximal end side A2 (inner surface side of the forceps piece 6B) from the upper end of the first tooth surface 641a toward the upper side B2, and at the uppermost end, it is connected to the inner peripheral surface 6b of the forceps piece 6B. The tissue T can be cut a cutting edge 642 formed at the intersecting ridge line between the inner peripheral surface 6b and the second tooth surface 741b.

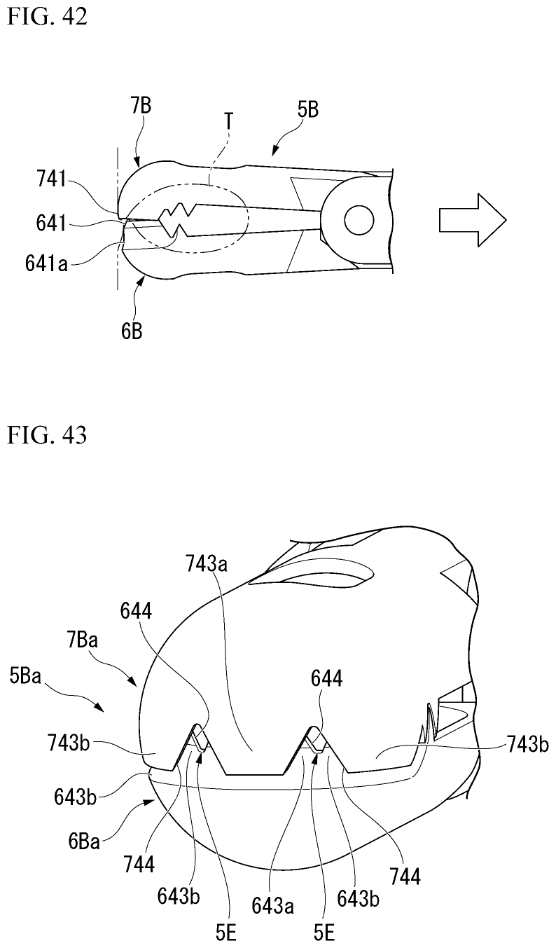

FIG. 42 is a diagram showing a state in which the tissue T is grasped by the forceps 5B according to the second embodiment.

In the present embodiment, as shown in FIG. 42, when the forceps piece 7B and the forceps piece 6B are closed, the forceps 5B can be pulled while pressing the tissue T by sandwiching the tissue T between the tooth surface 741a of the first scissors tooth 741 and the first tooth surface 641a and the second tooth surface 641b of the second scissors tooth 641. Therefore, it is easy to make a cut in the surface of the tissue T in the first scissors tooth 741 and the cutting edge 742.

Further, since the first scissors tooth 741 can be further bitten into the tissue T from the cut portion, the grasped tissue T is easily torn when the forceps 5B is pulled toward the proximal end side A2 (in the direction of the arrow in the figure). Thereby, the target tissue T can be easily collected with a small force.

The forceps pieces 7 and 6 of the present embodiment have sharp distal ends of the first scissors tooth 741 and the second scissors tooth 641, and both have sharp cutting edges 742 and 642. The first scissors tooth 741 may have a sharp cutting edge 742 because it is good to make a cut in the surface of the tissue T. On the other hand, the second scissors tooth 641 is not limited to the configuration in which the tooth tip has a sharp cutting edge 642, and the tooth tip of the second scissors tooth 641 does not have to be sharp.

The forceps 5B in this embodiment is not limited to the above configuration, and may have other structures.

Hereinafter, a modification of the forceps in the second embodiment will be described. In the following description, the same reference numerals will be given to the configurations common to those already described, and duplicate description will be omitted.

In each of the drawings below, in order to make each component easier to see, the scale of dimensions may be different depending on the component.

### (Modification 2-1)

In the second embodiment, the first scissors tooth 741 and the second scissors tooth 641 provided on the forceps pieces 7 and 6 are formed in an arc shape over the entire or predetermined range of arc-shaped open ends at the distal end portions 71A and 61A of the forceps cups 71 and 61, but it is not limited to the above-mentioned shape.

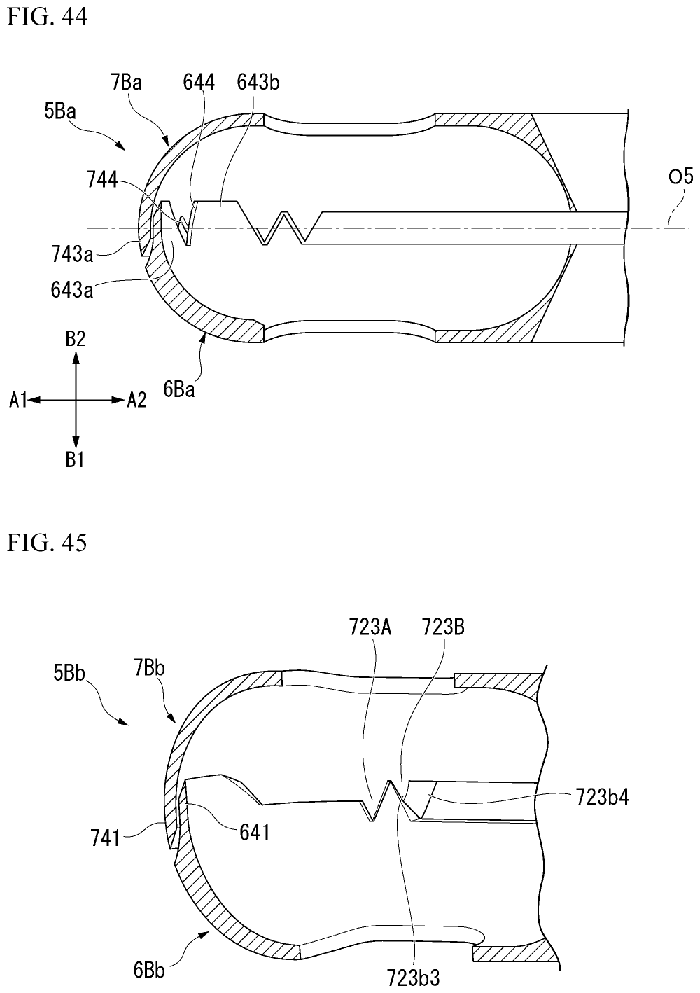

FIG. 43 is a perspective view of a forceps 5Ba in the modification 2-1 as viewed from the distal end side A1. FIG. 44 is a partial cross-sectional view of the forceps 5Ba in the modification 2-1.

The forceps 5Ba of this example shown in FIG. 43 is configured by including a forceps piece (first gripping piece) 7Ba having a plurality of scissors teeth 743a and 743b, and a forceps piece (second gripping piece) 6Ba having a plurality of scissors teeth 643a and 643b.

In the forceps piece 7Ba, three scissors teeth 743a and 743b and two V grooves 744 are alternately formed on the opening edge of the forceps cup 71. Specifically, the scissors teeth 743b are formed on the left and right sides of the scissors teeth 743a located on the central axis O5 via the V groove 744.

In the forceps piece 6Ba, three scissors teeth 643a and 643b and two V grooves 644 are alternately formed on the opening edge of the forceps cup 61. Specifically, the scissors teeth 643b are formed on the left and right sides of the scissors teeth 643a located on the central axis O5 via the V groove 644.

When the forceps 5Ba is closed, the three scissors teeth 743a and 743b of the forceps piece 7Ba are arranged on the distal end side A1 of the three scissors teeth 643a and 643b of the forceps piece 6Ba, the scissors teeth 743a of the forceps piece 7Ba and the scissors teeth 643a of the forceps piece 6Ba located on the central axis O5 overlap each other, and the pair of scissors teeth 743b of the forceps piece 7Ba and the pair of scissors teeth 643b of the forceps piece 6Ba overlap each other.

In the plurality of V grooves 744 and 644 provided in the forceps pieces 7Ba and 6Ba, a part of the forceps pieces 7Ba and 6Ba communicate with each other even when the forceps pieces 7Ba and 6Ba are in a completely closed state, and partially open in the longitudinal direction A. In this way, two gaps E5 are formed on the distal end side A1 of the forceps 5Ba, but the size thereof is not limited to the indicated size. The size of the gap E5 can be appropriately changed depending on the groove depth of the V grooves 744 and 644, the degree of overlap of the forceps pieces 7Ba and 6Ba, and the like.

According to this example, when the tissue T is grasped by the forceps 5Ba, by compressing a part of the tissue T that has entered the gap E5 formed by the V grooves 744 and 644 and rubbing the opposing scissors teeth 743a and 643a and the scissors teeth 743b and 643b against each other, it is possible to make a cut in the tissue T by the scissors teeth 743a and 743b and the scissors teeth 643a and 643b. By providing the V grooves 744 and 644, the edges of the scissors teeth 743a and 743b and the scissors teeth 643a and 643b are effective to easily bite into the tissue T, and a notch can be easily made in the tissue T at the distal end side A1 of the forceps 5Ba.

### (Modification 2-2)

The plurality of protruding teeth shown in the previous embodiment have a substantially constant thickness in the longitudinal direction A, but the thickness is not limited to this. For example, in at least one protruding tooth among the plurality of protruding teeth, the thickness of the protruding tooth may change in the longitudinal direction A.

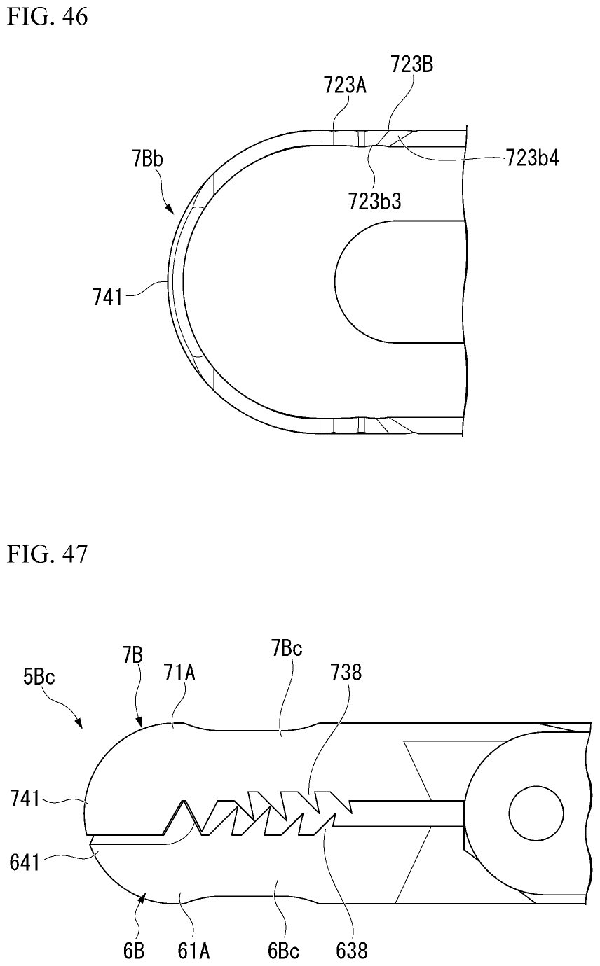

FIG. 45 is a partial cross-sectional view showing a forceps 5Bb in the modification 2-1. FIG. 46 is a bottom view showing a forceps piece (first gripping piece) 7Bb of the forceps 5Bb in the modification 2-1.

In the forceps 5Bb shown in FIGS. 45 and 46, for example, the thickness of the second distal end protruding tooth 723B of the forceps piece 7Bb is changed in the longitudinal direction A. Specifically, in the plan view shown in FIG. 46, the second distal end protruding tooth 723B has an inner surface 723b3 continuous with the inner peripheral surface 71a of the forceps cup 71, and an inner inclined surface 723b4 inclined at a predetermined angle with respect to the central axis O5. The inner inclined surface 723b4 is inclined in a direction away from the central axis O5 (outward in the radial direction) from the distal end side A1 to the proximal end side A2. That is, the wall thickness of the second distal end protruding tooth 723B gradually decreases from a predetermined position on the distal end side A1 toward the proximal end side A2, and the proximal end side A2 has a sharp edge shape.

According to the forceps 5Bb in this example, since the proximal end side A2 of the second distal end protruding tooth 723B is sharpened, a cut can be made in the grasped target tissue T, and the target tissue T is easily torn. Therefore, the target tissue T can be easily collected with a small force.

### (Modification 2-3)

FIG. 47 is a right side view partially showing the forceps 5Bc of the modification 2-3 in the second embodiment.

As shown in FIG. 47, a plurality of return teeth 638 and 738 may be provided on forceps pieces 6Bc and 7Bc. By providing a plurality of return teeth 638 and 738 inclined from the distal end side A1 toward the proximal end side A2 at substantially the center of the forceps pieces 6Bc and 7Bc in the longitudinal direction A, the return teeth 638 and 738 bite into the target tissue T grasped by the second scissors tooth 641 and the first scissors tooth 741 provided on the distal end portions 61A and 71A when the grasped target tissue T slides out to the distal end side A1, so that the target tissue T can be fixed near the center of the forceps pieces 6 and 7 in the length direction, and slipping to the distal end side A1 can be suppressed.

### Third Embodiment

An endoscopic treatment tool 100C according to the third embodiment of the present disclosure will be described with reference to the drawings. In the following description, the same reference numerals will be given to the configurations common to those already described, and the description thereof will be omitted.

[Endoscopic treatment tool 100C]

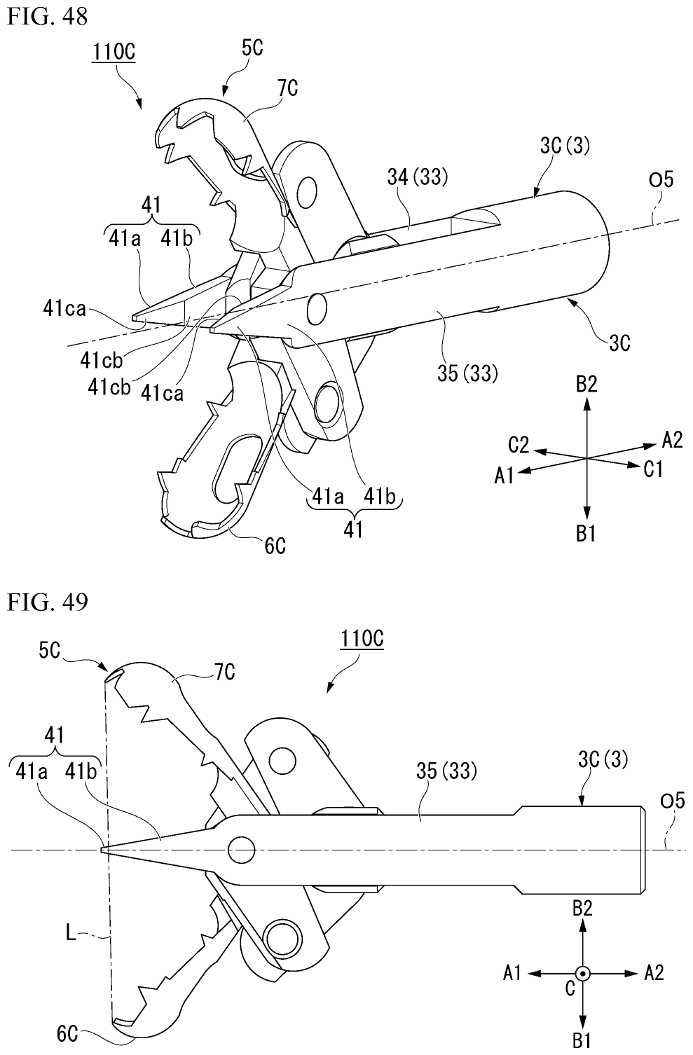

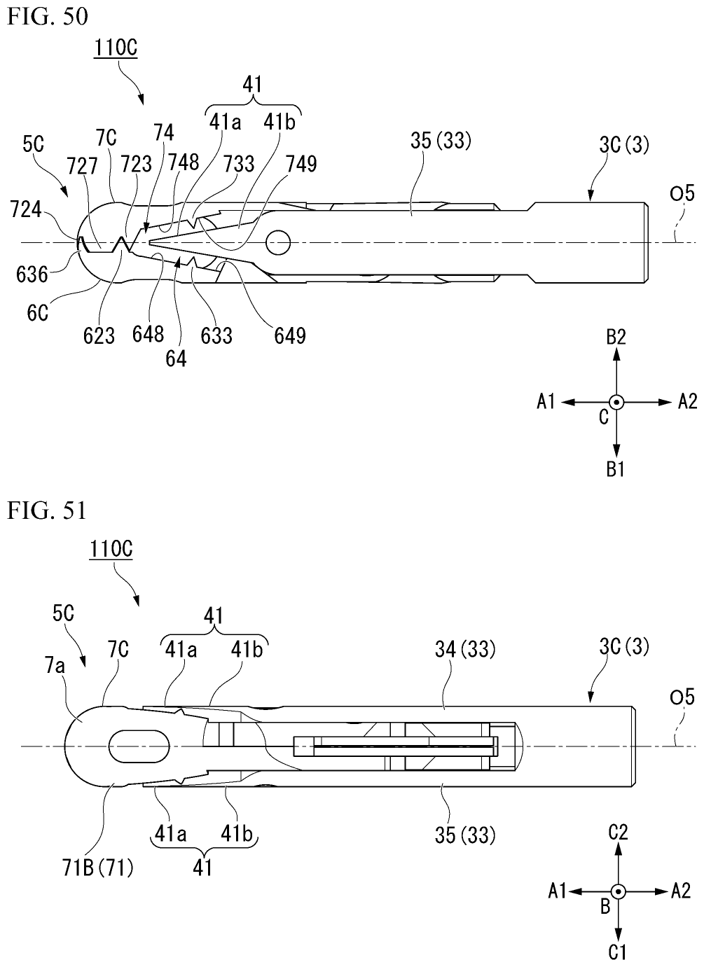

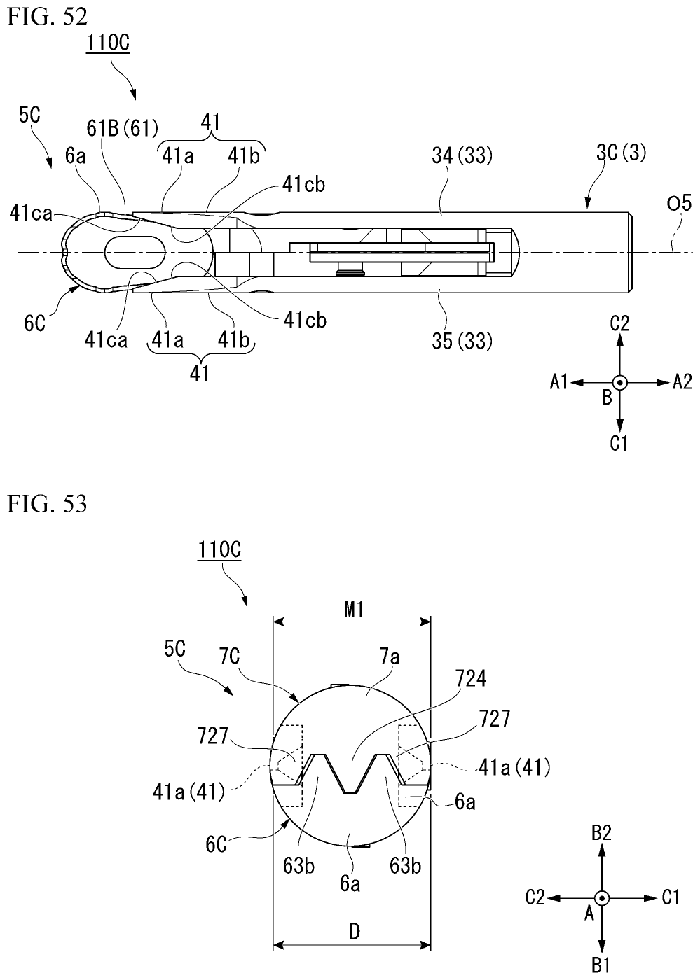

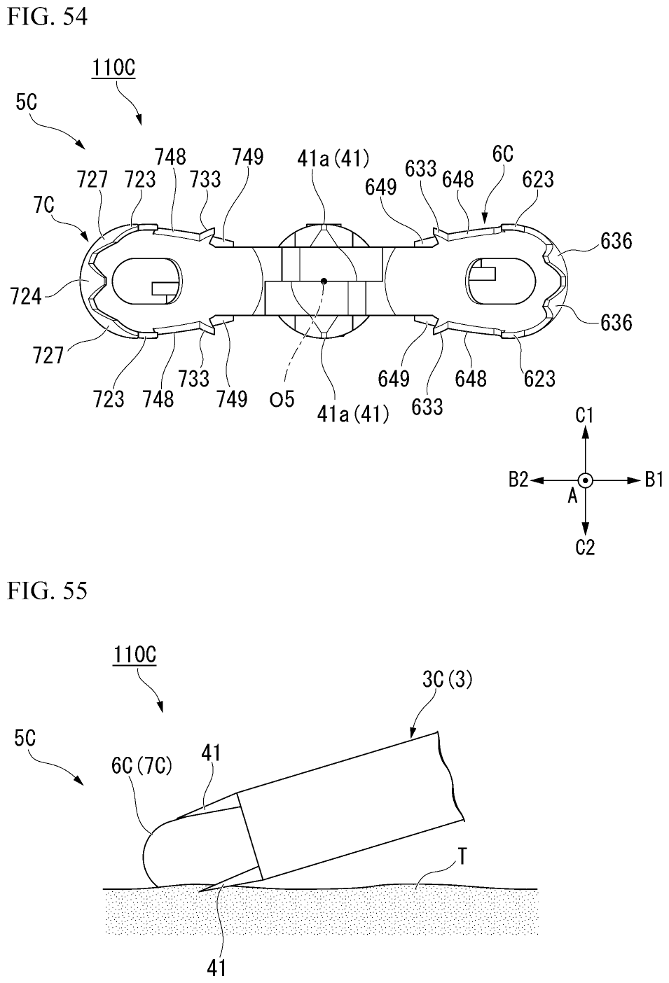

FIG. 48 is a perspective view showing an open state of a forceps 5C in a treatment portion 110C according to the third embodiment. FIG. 49 is a side view showing an open state of the forceps 5C in the treatment portion 110C according to the third embodiment. FIG. 50 is a side view showing the closed forceps 5C in the treatment portion 110C according to the third embodiment. FIG. 51 is a plan view showing the closed forceps 5C in the treatment portion 110C according to the third embodiment. FIG. 52 is a view showing the forceps 5C (forceps piece 6C) in the treatment portion 110C according to the third embodiment, and is a plan view omitting the illustration of the forceps piece (first gripping piece) 7C. FIG. 53 is a front view of the closed forceps 5C in the treatment portion 110C according to the third embodiment. FIG. 54 is a front view of the open forceps 5C in the treatment portion 110 according to the third embodiment.

The endoscopic treatment tool 100C of the third embodiment shown in FIG. 1 (simply also referred to as the treatment tool 100C) is used as an endoscopic treatment system together with the endoscope 200 like the endoscopic treatment tool 100 of the first embodiment. The treatment tool 100C includes a sheath 1, an operation wire 2, a support member 3, the forceps 5C, and an operating portion 8.

The forceps 5C of the present embodiment is different from the configurations of the first embodiment and the second embodiment in that a support member 3C has an outward needle (needle) 41.

As shown in FIGS. 48 to 54, the endoscopic treatment tool 100C of the present embodiment includes the support member 3C having a pair of outward needles 41 on the distal end side A1. As shown in FIGS. 48, 49 and 50, the pair of outward needles 41 are provided so as to protrude from the pair of frame pieces 33 (first frame piece 34 and second frame piece 35) to the distal end side A1. The pair of outward needles 41 face each other in the left-right direction via the central axis O5.

As shown in FIGS. 50 to 52, the pair of outward needles 41 includes a base portion 41b connected to each frame piece 33 (first frame piece 34, second frame piece 35), and a sharp portion 41a having a sharp distal end side A1. As shown in FIG. 50, the width of the outward needle 41 in the vertical direction B orthogonal to the central axis O5 is narrower than the width of the frame pieces 34 (33) and 35 (33), and gradually narrows (thinners) from the base portion 41b to the sharp portion 41a. The sharp portion 41a is used for treating living tissue.

An inner surface 41cb of each base portion 41b in the pair of outward needles 41 is a surface parallel to the central axis O5 and face each other via the central axis O5. An inner surface 41ca of each sharp portion 41a of the pair of outward needles 41 is inclined outward in the radial direction from the proximal end side A2 (base portion 41b) toward the distal end side A1. That is, the sharp portion 41a of the outward needle 41 in the present embodiment becomes thinner in the radial direction from the proximal end side A2 to the distal end side A1, and has an outward needle shape. In this way, by reducing the width and thickness of the sharp portion 41a toward the distal end side A1, the distal end side A1 (sharp portion 41a) of the outward needle 41 is formed into a sharp shape that easily pierces the living tissue.

As shown in FIGS. 48 and 49, when the forceps 5C is in the open state, the outward needle 41 is exposed between the forceps pieces 6C and 7C. As shown in FIG. 50, when the forceps 5C is in the closed state, the outward needle 41 is housed in the area surrounded by the forceps pieces 6C and 7C. Therefore, the forceps pieces 6C and 7C of the present embodiment have an outer shape corresponding to the shape of the outward needle 41. The forceps pieces 6C and 7C will be described later.

When the forceps 5C is fully open as shown in FIG. 49, the length of the outward needle 41 in the longitudinal direction is such that the distal end of the sharp portion 41a protrudes slightly toward the distal end side A1 from the straight line L connecting the distal ends of the forceps piece (first gripping piece) 7C and the forceps piece (second gripping piece) 6C. Not limited to this, for example, the length of the outward needle 41 in the longitudinal direction A may be such that the distal end of the sharp portion 41a coincides with the straight line L.

As shown in FIGS. 51, 52, and 53, the distal end of the outward needle 41 (sharp portion 41a) has the maximum outer diameter of the forceps pieces 6C and 7C (forceps cups 61 and 71) when the forceps 5C is closed. It is located at the same position as the outer peripheral surface 7a. That is, the maximum outer diameter D of the forceps cups 61 and 71 and the distance M1 between the sharp portions 41a of the pair of outward needles 41 facing each other in the radial direction satisfy the relationship of D=M1.

Further, in the previous embodiment, in the plan view shown in FIGS. 51 and 52, the semi-cylindrical portions 61B and 71B of the forceps cups 61 and 71 were formed with a constant width in the longitudinal direction A, but in the embodiment, as shown in FIGS. 51 and 54, the width of the semi-cylindrical portions 61B and 71B in the width direction C gradually narrows from the distal end side A1 (distal end portions 61A and 71A) in the longitudinal direction A to the proximal end side A2.

When the forceps 5C is in the closed state, the notch portions 64 and 74 face each other in the vertical direction, and the outward needle 41 is arranged in the area surrounded by the notch portions 64 and 74. A space along the outer shape of the outward needle 41 is formed by these notch portions 64 and 74, and it is possible to prevent the proximal end side A2 of each forceps cup 61 and 71 from coming into contact with the outward needle 41.

The forceps piece 7C includes a most advanced protruding tooth 724, a pair of protruding portions 727, a pair of first inclined portions 748, a pair of protruding teeth 733, and a pair of second inclined portions 749. As shown in FIG. 50, the forceps piece 7C includes a notch portion (first notch portion) 74 that is notched at the open ends at the proximal end side A2 in the longitudinal direction A and on both sides of the semi-cylindrical portion 71B in the width direction, in a direction away from the forceps piece 6C. The first inclined portion 748 and the second inclined portion 749 forming the notch portion 74 are inclined in a direction away from the central axis O5 toward the upper side B2 as it goes from the distal end side A1 to the proximal end side A2, and when the forceps 5C is closed, they face parallel to an oblique side 42 of the upper side B2 of the outward needle 41.

The forceps piece 6C includes a pair of protruding teeth 636, a pair of protruding teeth 623, a pair of first inclined portions 648, a pair of proximal end protruding teeth 633, and a pair of second inclined portions 649. As shown in FIG. 50, the forceps piece 6C includes a notch portion (second notch portion) 64 that is notched at the proximal end side A2 in the longitudinal direction A and at the open ends on both sides of the semi-cylindrical portion 61B in the width direction in a direction away from the forceps piece 7C. The first inclined portion 648 and the second inclined portion 649 forming the notch portion 64 are inclined in a direction away from the central axis O5 toward the lower side B1 as they go from the distal end side A1 to the proximal end side A2, and when the forceps 5C is closed, they face parallel to the oblique side 42 of the lower side B1 of the outward needle 41.

When the forceps 5C is closed, that is, when the forceps pieces 7C and the distal end side A1 of the forceps pieces 6C are engaged with each other, the proximal end protruding teeth 733 and 633 of the forceps pieces 7C and 6C are slightly separated from the outward needle 41, so as to avoid contact with the outward needle 41.

According to the configuration of the present embodiment, when the target tissue T is grasped by the forceps 5C, if the forceps 5C is closed with at least one of the pair of outward needles 41 provided on the distal end side A1 of the support member 3C pressed (pierced) by the target tissue T, the tissue around the portion where the outward needle 41 is pressed is attracted to the inside of the forceps cups 61 and 71 and collected. By closing the forceps 5C while pressing or piercing the external needle 41 against the target tissue T in this way and fixing the forceps 5C, it is possible to suppress the forceps 5C from slipping against the tissue, and the forceps pieces 6C and 7C can be grasped without missing the target tissue T.

Further, the length of the outward needle 41 in the longitudinal direction A is longer than the length of the forceps cups 61 and 71 in the longitudinal direction A with the forceps 5C fully opened. That is, when the forceps 5C is fully open, the distal end of the outward needle 41 is slightly protruded toward the distal end side A1 in the longitudinal direction A from the straight line L connecting the distal ends of the forceps pieces 6C and 7C. Thereby, as shown in 55, when approaching the target tissue T from an oblique direction, the outward needle 41 can be inserted into the target tissue T. FIG. 55 is a schematic view showing the approach posture of the treatment portion 110C with respect to the target tissue T.

Further, the outward needle 41 is an outward needle in which the inner surface 41ca of the sharp portion 41a inclines from the inside to the outside in the radial direction toward the distal end side A1, and the distal end thereof substantially coincides with the outer peripheral surface having the maximum outer diameter of the forceps 5C. Therefore, since the outward needle 41 can be pressed against the target tissue T before or at the same time as the forceps pieces 6C and 7C, the outward needle 41 can be easily inserted into the target tissue T. Further, by using the outward needle 41, it is possible to prevent the volume of the forceps cups 61 and 71 from being significantly reduced due to the needle structure, so that the amount of tissue collected can be secured.

Since the outward needle 41 is not configured to protrude radially outward from the outer peripheral surface which is the maximum outer diameter of the forceps 5C, when the endoscopic treatment tool 100C is inserted into the channel 216, the inside of the channel 216 is not easily damaged. Further, when the treatment tool 100 is moved in the body, the outward needle 41 is less likely to damage the living tissue.

Further, since the pair of frame pieces 33 in the support member 3C and the outward needle 41 extending from each distal end are integrally formed, it is possible to suppress an increase in the overall width of the forceps 5C as compared with a configuration in which a needle member separate from the support member 3 is provided.

### (Modification 3-1)

In the third embodiment, the support member 3C has an outward needle 41, but the direction of the needle is not limited to this.

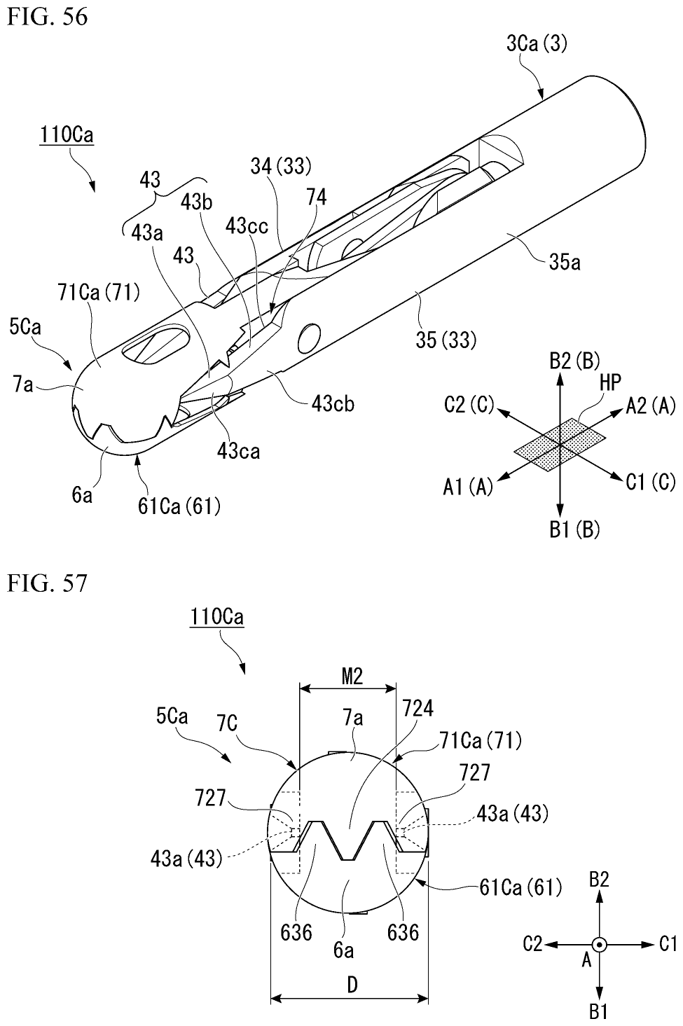

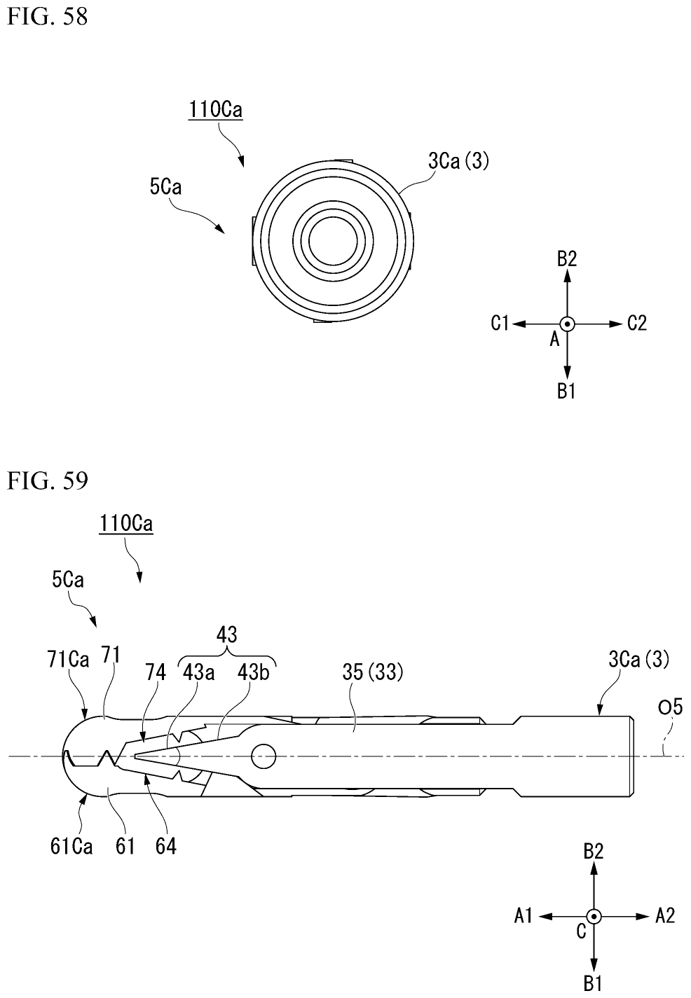

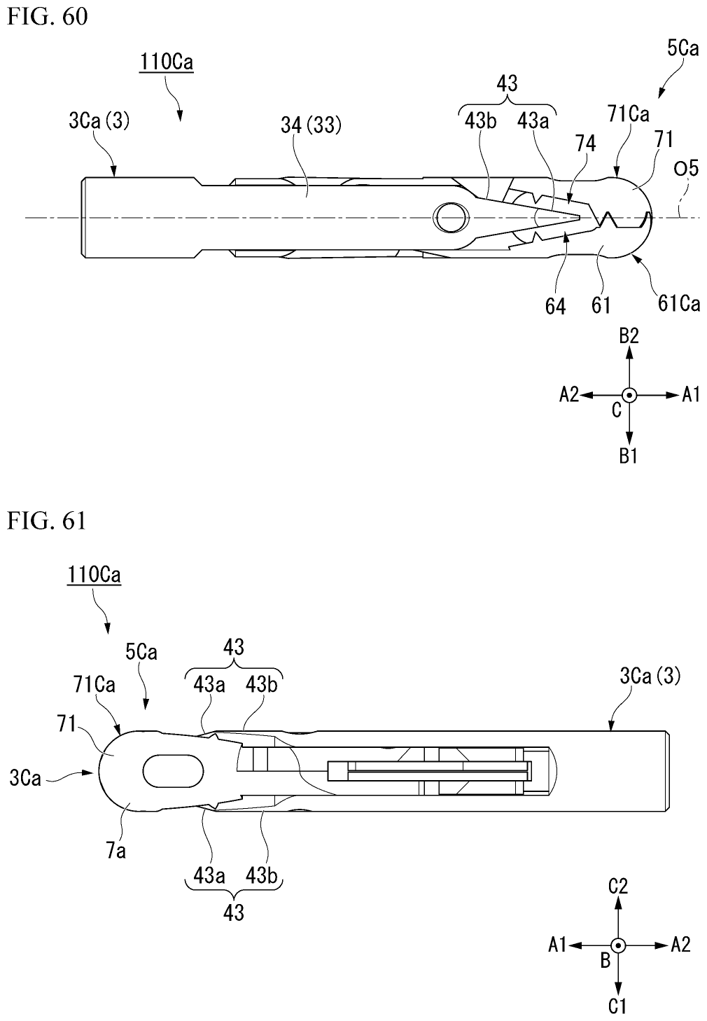

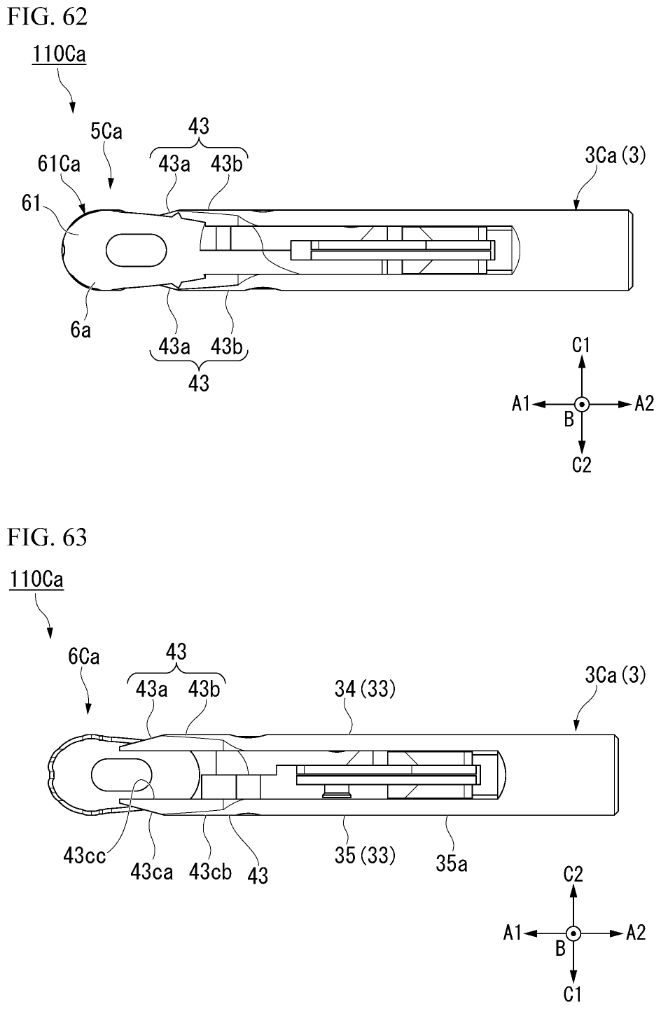

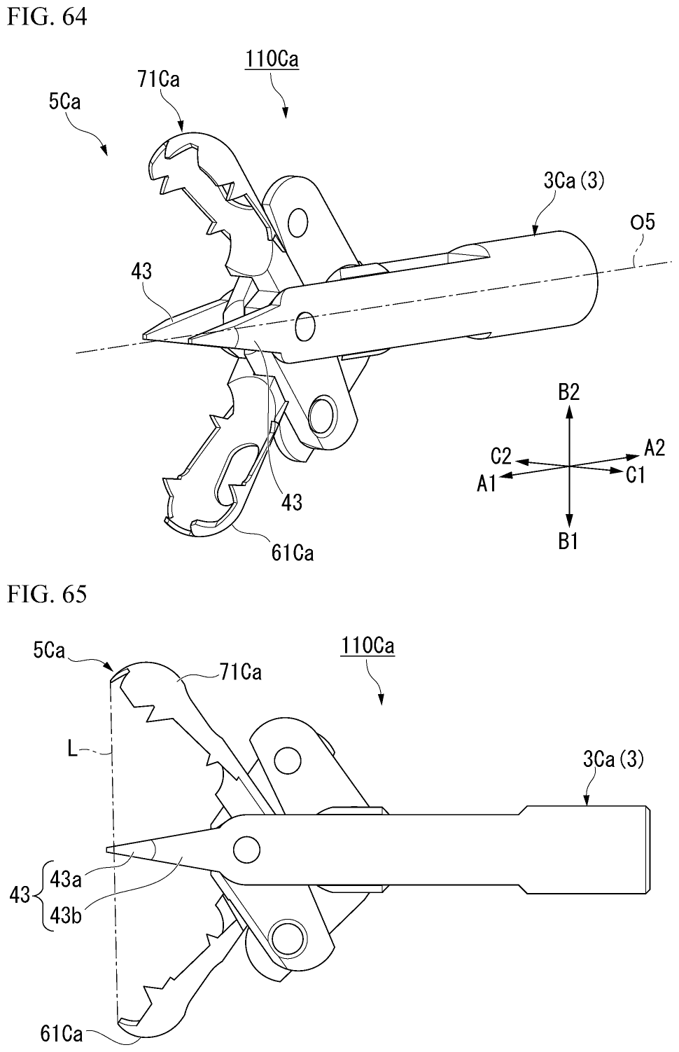

FIG. 56 is a perspective view showing a closed state of a forceps 5Ca of the modification 3-1 in a treatment portion 110Ca. FIG. 57 is a front view showing a closed state of the forceps 5Ca of the modification 3-1 in the treatment portion 110Ca. FIG. 58 is a rear view showing a closed state of the forceps 5Ca of the modification 3-1 in the treatment portion 110Ca. FIG. 59 is a right side view showing the closed state of the forceps 5Ca of the modification 3-1 in the treatment portion 110Ca. FIG. 60 is a left side view showing a closed state of the forceps 5Ca of the modification 3-1 in the treatment portion 110Ca. FIG. 61 is a plan view showing a closed state of the forceps 5Ca of the modification 3-1 in the treatment portion 110Ca. FIG. 62 is a bottom view showing a closed state of the forceps 5Ca of the modification 3-1 in the treatment portion 110Ca. FIG. 63 is a view showing the forceps 5Ca (forceps piece 6Ca) of the modification 3-1 in the treatment portion 110Ca, and is a plan view showing the forceps piece 7Ca. FIG. 64 is a perspective view showing the forceps 5Ca of the modification 3-1 in the treatment portion 110Ca. FIG. 65 is a right side view showing the forceps 5Ca of the modification 3-1 in the treatment portion 110Ca.

The forceps 5Ca of this example shown in FIG. 56 includes a support member 3Ca having a pair of introverted needles (needle) 43. As shown in FIGS. 56, 59, and 60, when the forceps 5Ca is in the closed state, the pair of introverted needles 43 are housed in the respective regions surrounded by the notch portions 64 and 74 of the forceps pieces 6Ca and 7Ca. As shown in FIGS. 63 and 64, when the forceps 5Ca is open, the pair of introverted needles 43 are exposed between the forceps pieces 6Ca and 7Ca.

As shown in FIGS. 56, 59, and 60, the pair of introverted needles 43 are provided so as to protrude from the pair of frame pieces 33 (first frame piece 34 and second frame piece 35) to the distal end side A1. The pair of introverted needles 43 face each other in the left-right direction via the central axis O5.

As shown in FIG. 56, the pair of introverted needles 43 includes a base portion 43b connected to each frame piece 33 (first frame piece 34, second frame piece 35), and a sharp portion 43a having a sharp distal end side A1. As shown in FIGS. 59 and 60, the width of the inward needle 43 in the vertical direction B orthogonal to the central axis O5 is narrower than the width of the frame pieces 34 (33) and 35 (33), and is gradually narrowed (thinner) from the base portion 43b to the sharp portion 43a. The sharp portion 43a is used for treating living tissue.

As shown in FIG. 63, inner surfaces 43cc of the pair of inward needles 43 are planes parallel to the central axis O5 and face each other via the central axis O5. An outer surface 43ca of each sharp portion 43a of the pair of inward needles 43 is inclined inward in the radial direction from the proximal end side A2 (base portion 43b) toward the distal end side A1. That is, the sharp portion 43a of the inward needle 43 in the present embodiment becomes thinner in the radial direction from the proximal end side A2 to the distal end side A1, and has an inward needle shape. In this way, by reducing the width and thickness of the sharp portion 43a toward the distal end side A1, the distal end side A1 (sharp portion 43a) of the introverted needle 43 is formed into a sharp shape that easily penetrates the living tissue.

As shown in FIG. 65, the length of the introverted needle 43 in the longitudinal direction is such that the distal end of the sharp portion 43a protrudes slightly toward the distal end side A1 from the straight line L connecting the distal ends of the forceps piece 7Ca and the forceps piece 6Ca when the forceps 5Ca is in a fully open state. Not limited to this, for example, the length of the inward needle 43 in the longitudinal direction A may be such that the distal end of the sharp portion 43a coincides with the straight line L.

As shown in FIGS. 63 and 64, when the forceps 5C is in the closed state, the distal end of the inward needle 43 (sharp portion 43a) is located inside the forceps cups 61 and 71 closer to the central axis O5 than the outer peripheral surface which is the maximum outer diameter D. That is, the maximum outer diameter D of the forceps cups 61 and 71 and the distance M2 between the sharp portions 43a of the pair of inward needles 43 facing each other in the radial direction satisfy the relationship of D>M2.

According to the configuration of this example, by using the introverted needle 43, the distal end of the introverted needle 43 can be accommodated inside the forceps cups 61 and 71 when the forceps 5Ca is closed. By covering the distal end of the introverted needle 43 with the forceps cups 61 and 71 and hiding inside the forceps cups 61 and 71, the treatment portion 100Ca can be smoothly passed through the treatment portion 100Ca in the channel 216 shown in FIG. 5. Further, when the forceps cups 61 and 71 are bent or displaced by passing the treatment portion 100Ca through the channel 216, the needle distal end may pop out from the forceps cups 61 and 71, but damage to the coil sheath 11 can be minimized.

(Modification example 3-2)

In the third embodiment, the support members 3C and 3Ca having the outward needle 41 or the inward needle 43 can be adopted regardless of the shape of the forceps pieces 6C and 7C (forceps cups 61 and 71). That is, a modification 3-2 of the present embodiment may have a configuration including the support member 3 (3C, 3Ca) having the outward needle 41 or the inward needle 43, and the forceps 5, 5Aa, 5Ab, 5Ad, 5B, 5Ba which are the first embodiment, the second embodiment, and the modification of each embodiment described above. At this time, it may be good to provide notch portions 64 and 74 in each of the forceps cups 61 and 71 so that the outward needle 41 or the inward needle 43 is arranged between them.

### Fourth Embodiment

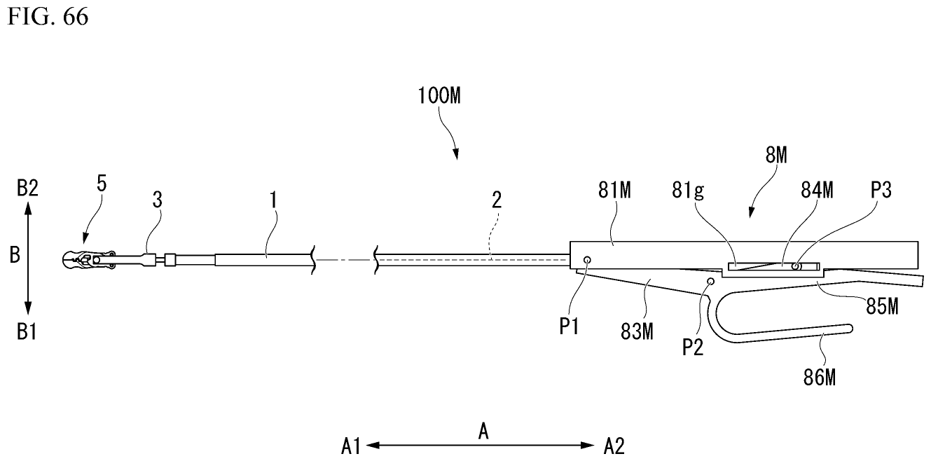

An endoscopic treatment tool 100M according to the fourth embodiment of the present disclosure will be described with reference to FIGS. 66 to 71. In the following description, the same reference numerals will be given to the configurations common to those already described, and duplicate description will be omitted.

[Endoscopic treatment tool 100M]

FIG. 66 is an overall view showing the endoscopic treatment tool 100M.

The endoscopic treatment tool 100M (also referred to as a treatment tool 100M) is used as an endoscopic treatment system together with the endoscope 200 like the endoscopic treatment tool 100 of the first embodiment. The treatment tool 100M includes a sheath 1, an operation wire 2, a support member 3, a forceps (jaw) 5, and an operating portion 8M.

[Operating portion 8M]

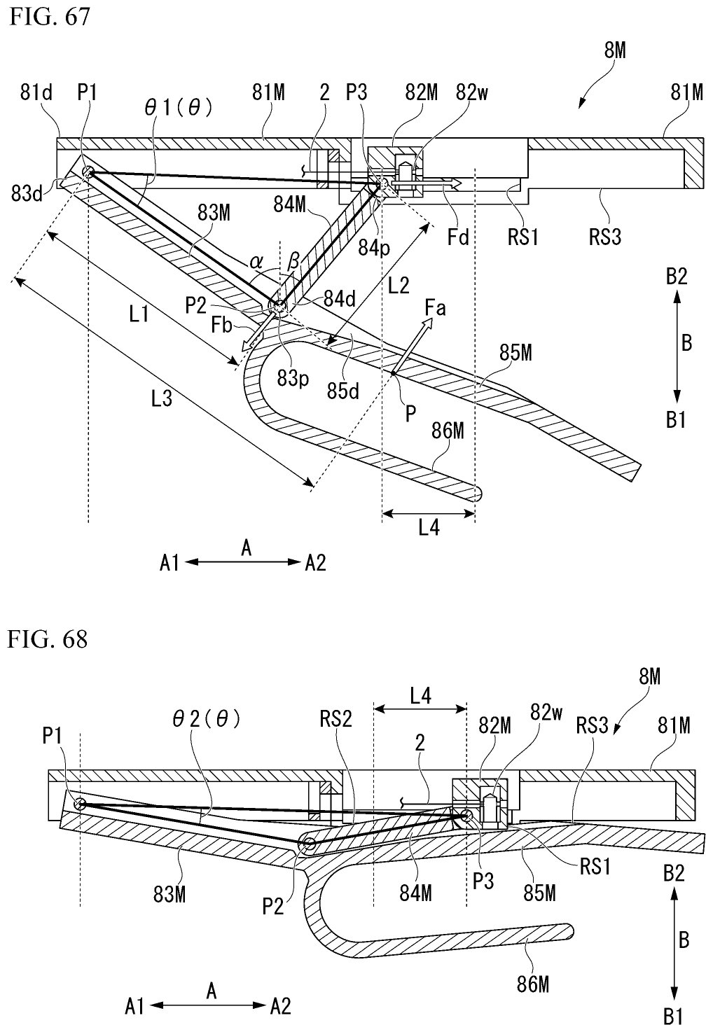

FIGS. 67 and 68 are cross-sectional views of the operating portion 8M.

The operating portion 8M is provided on the proximal end side A2 of the sheath 1. The operating portion 8M includes a housing (operating portion body) 81M, a slider 82M, a trigger (first link) 83M, a rod (second link) 84M, a handle 85M, a cover 86M, a first connecting pin P1, and a second connecting pin P2.

The housing 81M is a substantially box-shaped member extending in the longitudinal direction A. A slider 82M is attached to the housing 81M so as to be slidable in the longitudinal direction A. As shown in FIG. 66, the housing 81M has guide rails 81g extending in the longitudinal direction A on both sides in the width direction C.

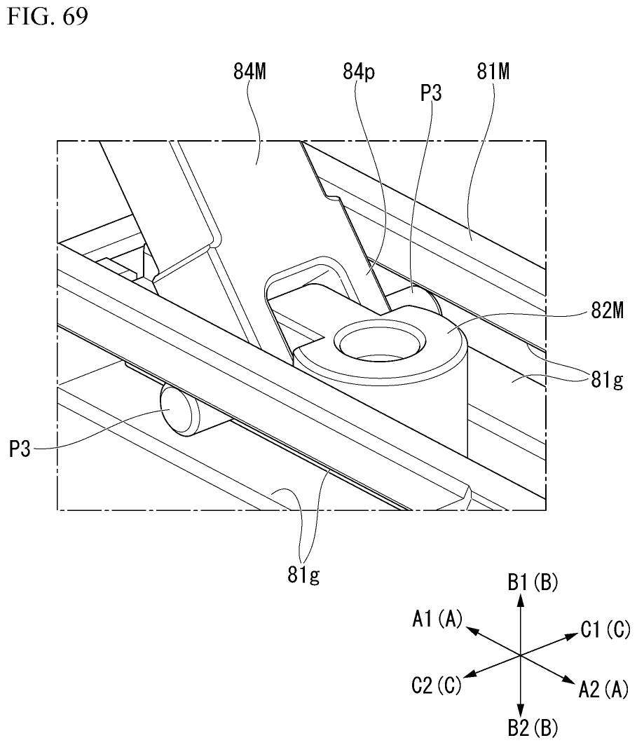

FIG. 69 is a perspective view of the slider 82M seen from the lower side B1 in the operating portion 8M.

The slider 82M has a wire-fixing portion 82w and slide pins (third connecting pins) P3 extending on both sides in the width direction C. The slider 82M is attached to an intermediate portion of the housing 81M in the longitudinal direction A so as to be slidable in the longitudinal direction A.

As shown in FIG. 69, a slide pin P3 can be slidably moved in the longitudinal direction A by engaging with a guide rail 81g formed on the housing 81M. As shown in FIG. 67, the proximal end of the operation wire 2 is attached to the wire-fixing portion 82w. As the slider 82M moves back and forth with respect to the housing 81M in the longitudinal direction A, the operation wire 2 moves back and forth with respect to the sheath 1.

The trigger (first link) 83M is a rod-shaped member made of metal or the like. A distal end 83d of the trigger (first link) 83M is rotatably attached to a distal end 81d of the housing 81M by the first connecting pin P1. The central axis of the first connecting pin P1 extends in the width direction C, and the trigger 83M rotates with the first connecting pin P1 as a fulcrum in a plane perpendicular to the width direction C with respect to the housing 81M (hereinafter, also referred to as a “movable plane”).

The rod (second link) 84M is a rod-shaped member made of metal, resin, or the like. A distal end portion 84d of the rod (second link) 84M is rotatably attached to a proximal end portion 83p of the trigger 83M by the second connecting pin P2. The central axis of the second connecting pin P2 extends in the width direction C, and the rod 84M rotates in a movable plane perpendicular to the width direction C with respect to the trigger 83M.

A proximal end portion 84p of the rod (second link) 84M is rotatably attached to the slider 82M by the slide pin (third connecting pin) P3 of the slider 82M. The central axis of the slide pin P3 extends in the width direction C, and the rod 84M rotates in a movable plane perpendicular to the width direction C with respect to the slider 82M.

The handle 85M is a thin plate member made of metal, resin, etc., and is a part on which the user hangs a finger. A distal end portion 85d of the handle 85M is connected to the proximal end portion 83p of the trigger 83M. The handle 85M is connected to the trigger 83M so that the longitudinal axis substantially coincides with the trigger 83M. In the present embodiment, the handle 85M and the trigger 83M are integrally formed.

The cover 86M is a thin plate member provided on the lower side B1 of the handle 85M, and is a surface that receives the user's finger when the user operates the trigger 83M in the lower side B1 direction.

[Toggle mechanism]

The housing 81M, the slider 82M, the trigger 83M, and the rod 84M form a toggle mechanism which is a kind of boosting mechanism. The operation of the toggle mechanism will be described below.

As shown in FIG. 67, the length from the first connecting pin P1 to the second connecting pin P2 is defined as the length L1. The length from the second connecting pin P2 to the slide pin P3 is defined as the length L2. The portion where the force is applied to the handle 85M by the user's finger is defined as the force point P, and the length from the first connecting pin P1 to the force point P is defined as the length L3. These lengths are the lengths on any movable plane.

As shown in FIG. 67, the angle formed by the direction perpendicular to the movable plane and the direction parallel to the trigger 83M is defined as the angle α, and the angle formed by the direction perpendicular to the movable plane and the direction parallel to the rod 84M is defined as the angle β. Further, the angle formed by the longitudinal direction of the housing 81M and the longitudinal direction of the trigger 83M on the movable plane is defined as the angle θ.

When a force Fa that closes the handle 85M with respect to the housing 81M acts on the force point P in the direction perpendicular to the handle 85M, the force Fb acting on the second connecting pin P2 in the direction parallel to the rod 84M is represented by Equations 1 to 3 from the balance of the moments in the first connecting pin P1. Here, X1 is the “lever ratio”.

$$\begin{matrix}[ Equation 1 ] & \\ Fa \times L 3 - Fb sin ( α + β ) \times L 1 = 0 & ( Equation 1 )\end{matrix}$$

$$\begin{matrix}[ Equation 2 ] & \\ X 1 = \frac{L 3}{L 1} & ( Equation 2 )\end{matrix}$$

$$\begin{matrix}[ Equation 3 ] & \\ Fb = \frac{1}{sin ( α + β )} \times X 1 \times Fa & ( Equation 3 )\end{matrix}$$

The force Fd acting on the proximal end side A2 in the longitudinal direction A, which is the force acting on the slide pin P3, is expressed by Equation 5 based on Equation 4 expressing the balance of the horizontal force on the slide pin P3. In Equation 5, the coefficient of Fa is called “power factor”.

$$\begin{matrix}[ Equation 4 ] & \\ Fb sin β - Fd = 0 & ( Equation 4 )\end{matrix}$$

$$\begin{matrix}[ Equation 5 ] & \\ Fd = Fb sin β = \frac{sin β}{sin ( α + β )} \times X 1 \times Fa & ( Equation 5 )\end{matrix}$$

The toggle mechanism can output a large force Fd from a small force Fa by appropriately setting the lever ratio X1 and the angles α and β. The slider 82M is moved to the proximal end side A2 by the force Fd output from the toggle mechanism, and can pull the operation wire 2.

The smaller the length L2, the more efficient the movement amount of the slider 82M with respect to the operation amount of the handle 85M, and the smaller the length L2 with respect to the length L1, the more efficient. For example, the length L1 is set to 40 mm to 100 mm, the length L2 is set to 20 mm to 100 mm, the maximum angle θ1 of the angle θ is set to 45 degrees, and the minimum angle θ2 of the angle θ is set to 10 degrees.

In the toggle mechanism, the “power factor” of the toggle mechanism increases as the slider 82M moves toward the proximal end side and the angle θ becomes smaller (see Equation 8). That is, the more the slider 82M moves to the proximal end side and pulls the operation wire 2, the greater the “power factor” of the toggle mechanism. When the forceps 5 is closed by pulling the operation wire 2, the “power factor” of the toggle mechanism increases as the forceps 5 is closed. Therefore, the user can perform, for example, a procedure of closing the forceps 5 and separating the tissue or the like with a small force.

When a force is applied to the handle 85M by the user's finger, the toggle mechanism is in the closed state shown in FIG. 68. When the toggle mechanism is in the closed state, the slider 82M contacts the first contact surface RS1 of the housing 81M, or the rod 84M contacts the second contact surface RS2 of the housing 81M, or the handle 85M contacts the third contact surface RS3 of the housing 81M. In the toggle mechanism, the movement range in the closing direction is restricted by the contact of the slider 82M, the rod 84M, and the handle 85M with any one of the contact surfaces RS. That is, the towing range of the operation wire 2 (moving range L4 of the slider 82M) is regulated by the moving range of the toggle mechanism.

The first contact surface RS1 is formed on the proximal end side A2 of the movement range L4 of the slider 82M in the housing 81M. As shown in FIG. 68, the slider 82M to which the proximal end side A2 has moved comes into contact with the first contact surface RS1 and cannot move from the first contact surface RS1 to the proximal end side A2. When the first contact surface RS1 is in contact, the second contact surface RS2 and the third contact surface RS3 are not in contact with each other.

The second contact surface RS2 is formed on the lower side B1 of the housing 81M. As shown in FIG. 68, the rod 84M approaching the housing 81M comes into contact with the second contact surface RS2 and cannot move to B2 above the second contact surface RS2. When the second contact surface RS2 is in contact, the first contact surface RS1 and the third contact surface RS3 are not in contact with each other.

The third contact surface RS3 is formed on the lower side B1 of the housing 81M. As shown in FIG. 68, the handle 85M approaching the housing 81M comes into contact with the third contact surface RS3 and cannot move to B2 above the third contact surface RS3. When the third contact surface RS3 is in contact, the first contact surface RS1 and the second contact surface RS2 do not contact.

With the above configuration, the operation will be normal even if there is a manufacturing error.

As for the toggle mechanism, the smaller the angle θ, the larger the “power factor” of the toggle mechanism. That is, as shown in FIG. 68, the closer the rod 84M and the handle 85M are to the housing 81M, the greater the “power factor” of the toggle mechanism. However, in the housing 81M, since the approaching rod 84M and the handle 85M come into contact with the housing 81M on the contact surface RS, the closing force that increases when the toggle mechanism is closed can be limited to an arbitrary force, and damage or destruction of the operating portion 8M can be prevented.

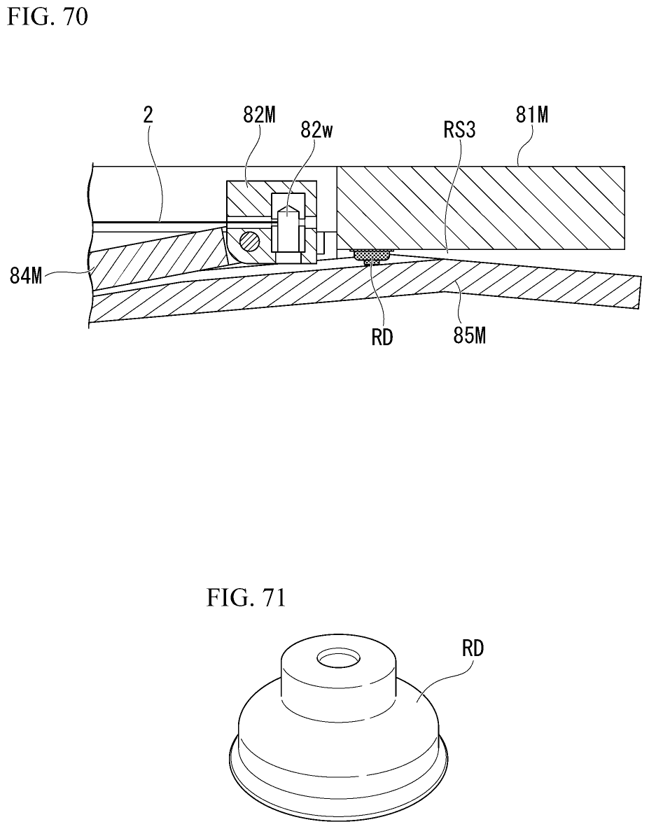

FIG. 70 is a cross-sectional view of the operating portion 8M equipped with the rubber dome RD.

The operating portion 8M may include the rubber dome RD illustrated in FIG. 71 in the housing 81M, for example, in the vicinity of the third contact surface RS3. As shown in FIG. 70, the handle 85M approaching the housing 81M comes into contact with the rubber dome RD immediately before coming into contact with the third contact surface RS3. The user can obtain tactile feedback by contacting the handle 85M with the rubber dome RD, and can recognize that the handle 85M will soon come into contact with the third contact surface RS3. Therefore, the user can operate the handle 85M after recognizing the movable range of the handle 85M.

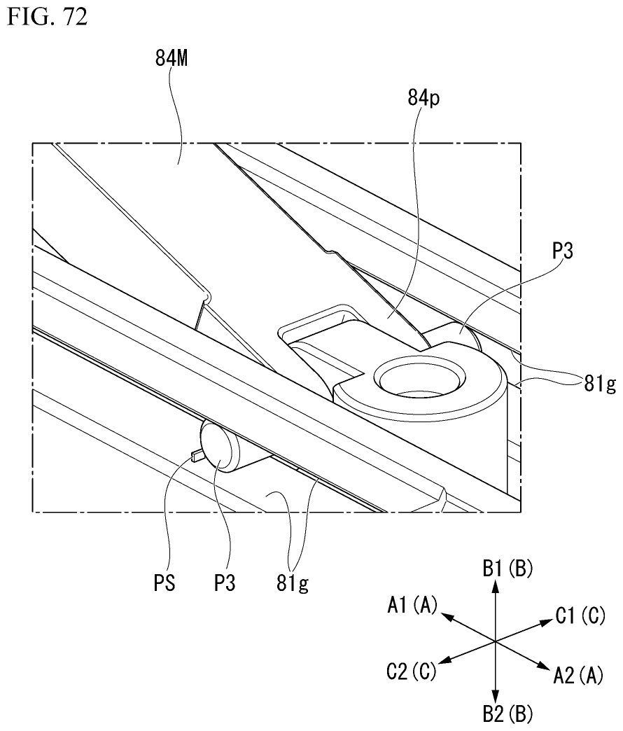

FIG. 72 is a perspective view of the slider 82M seen from the lower side B1 in the operating portion 8M equipped with the leaf spring PS.

The operating portion 8M may have a leaf spring PS in the housing 81M, for example, on the guide rail 81g. As shown in FIG. 72, the slide pin P3 of the slider 82M moved to the proximal end side A2 comes into contact with the leaf spring PS immediately before coming into contact with the first contact surface RS1. The user can obtain tactile feedback by contacting the slide pin P3 of the slider 82M with the leaf spring PS, and can recognize that the slider 82M will soon come into contact with the first contact surface RS1. Therefore, the user can operate the handle 85M after recognizing the movable range of the slider 82M.

According to the endoscopic treatment tool 100M according to the present embodiment, the operating portion 8M having a boosting mechanism is provided, and the operation wire 2 can be towed.

### Fifth Embodiment

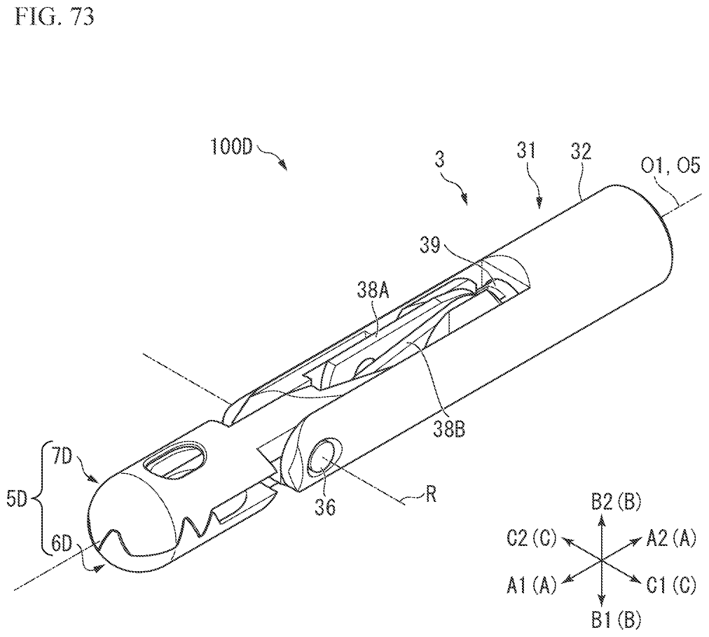

An endoscope treatment instrument 100D according to a fifth embodiment of the present disclosure will be described with reference to FIGS. 73 to 83. In the following description, the same reference numerals are given to the same configurations as those already described, and redundant descriptions will be omitted.

FIG. 73 is a perspective view of the forceps 5D of the endoscopic treatment instrument 100D according to the fifth embodiment.

The endoscopic treatment instrument 100D (simply referred to as the endoscopic treatment instrument 100D) is used as an endoscopic treatment system together with the endoscope 200 in the same manner as the endoscopic treatment instrument 100 of the first embodiment. The treatment instrument 100D includes a sheath 1, an operation wire 2, a support member 3, forceps 5D, and an operating portion 8.

[forceps (jaw) 5D]

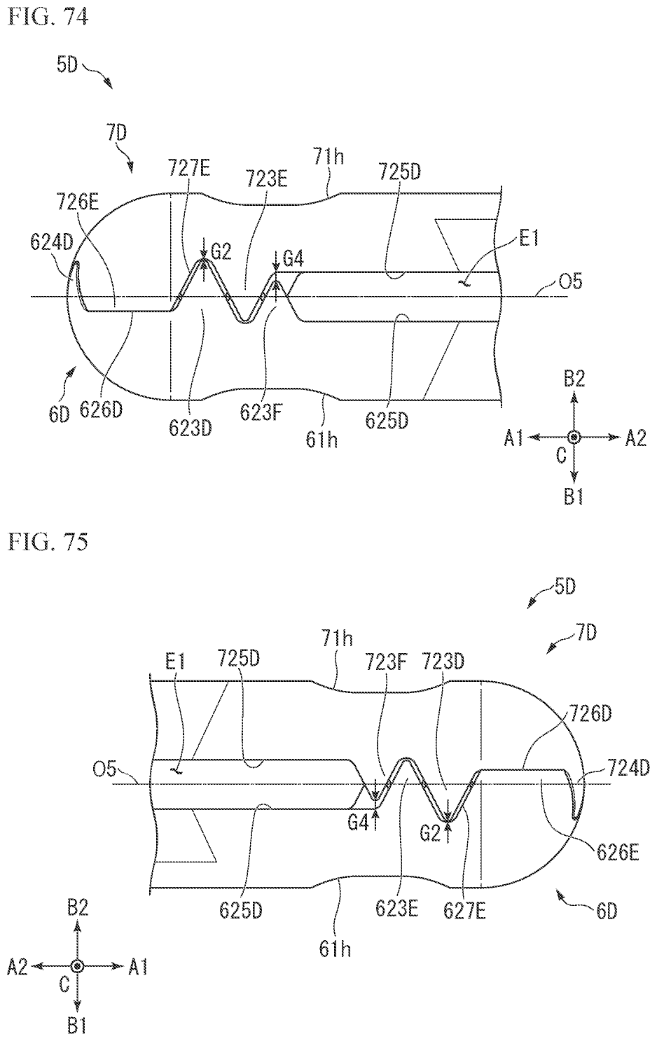

FIG. 74 is a right side view of the forceps 5D. FIG. 75 is a left side view of the forceps 5D.

A forceps (jaw) 5D is a member for collecting living tissue. The forceps 5D is made of a metal material such as stainless steel, and includes a forceps piece 6D and a forceps piece 7D that face each other in the closed state. When the forceps piece 6D and the forceps piece 7D are closed, the forceps 5D has a cylindrical shape. The forceps piece 6D and the forceps piece 7D are supported by a pin 36 so as to be capable of opening and closing toward the distal end side A1.

The forceps piece 6D is rotatably supported by the pin 36 with the rotation axis R of the pin 36 as the center of rotation. The forceps piece 6D is connected to the connection portion 39 via the support plate member 38A at the proximal end side A2 and is operated by the operation wire 2 connected to the connection portion 39.

The forceps piece 7D is rotatably supported by the pin 36 with the rotation axis R of the pin 36 as the center of rotation. The forceps piece 7D is connected to the connection portion 39 via the support plate member 38B at the proximal end side A2 and is operated by the operation wire 2 connected to the connection portion 39.

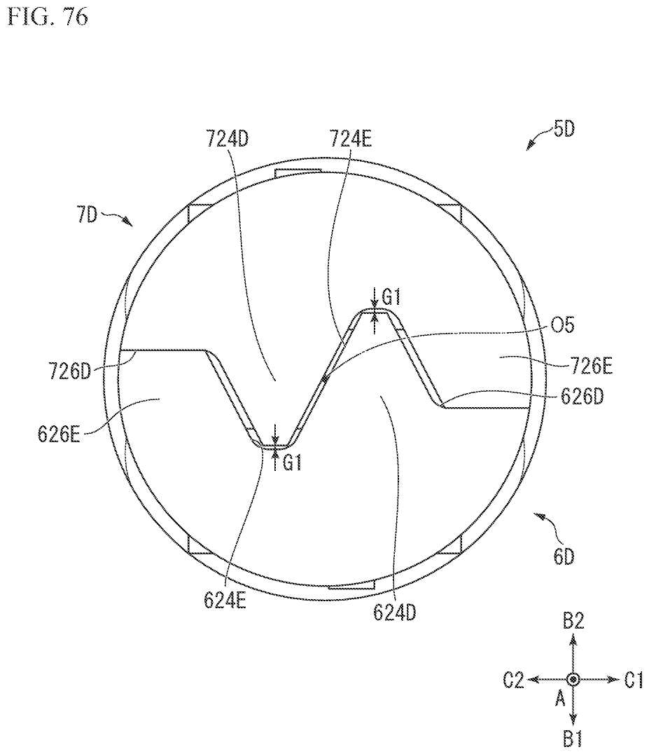

FIG. 76 is a front view of the forceps 5D.

The forceps piece 6D and the forceps piece 7D are arranged symmetrically with respect to the central axis O5 in the longitudinal direction A of the forceps 5D. A central axis O5 in the longitudinal direction A of the forceps 5D substantially coincides with a central axis O1 in the longitudinal direction A of the sheath 1.

One of the forceps piece 6D and the forceps piece 7D may be fixed to the support member 3, and only the other may be rotatably supported by the support member 3.

[Forceps piece (second forceps piece) 6D]

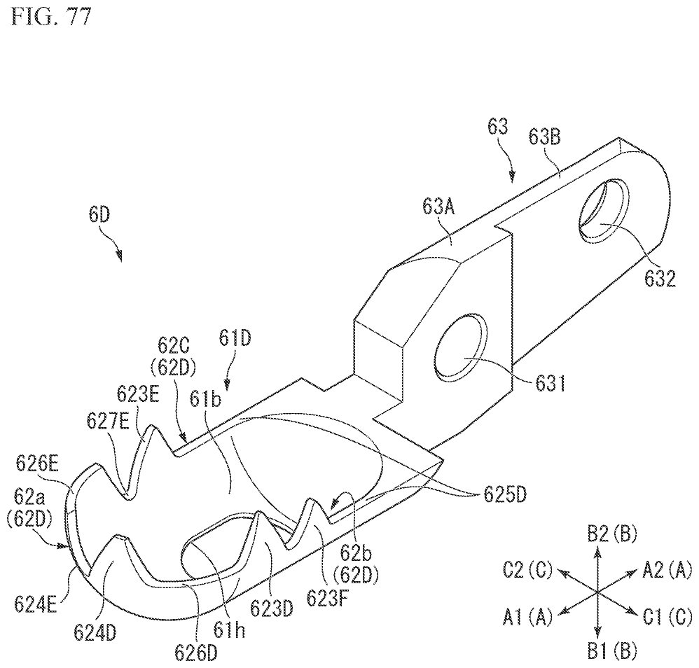

FIG. 77 is a perspective view of the forceps piece 6D.

The forceps piece (second gripping piece) 6D is formed, for example, mainly by cutting or pressing from one flat plate or one bar. The forceps piece 6D includes a forceps cup (second forceps cup) 61D provided on the distal end side A1 in the longitudinal direction A, and a first plate 63.

The forceps cup 61D is formed in a substantially hemispherical shape and opens toward the forceps piece 7D side (upper side B2) in the opening/closing direction B. The forceps cup 61D has an opening edge 62D and a through-hole 61h penetrating in the opening/closing direction B near the center of the concave bottom portion 61b.

The opening edge 62D is the opening edge of the forceps cup 61D, and is formed in a substantially arc shape in plan view. The opening edge 62D has a pair of first teeth 624D, recesses 626D, tip grooves 624E, protruding portions 626E, side grooves 627E, second teeth 623D, third teeth 623E, fourth teeth 623F, and a flat portion 625D.

The opening edge 62D is divided into a leading edge 62a that is an edge on the leading edge side A1 of the opening edge 62D, a first edge 62b that is an edge on the right side C1 (a first side in the width direction C) of the opening edge 62D, and a second edge 62c that is the edge on the left side C2 (a second side in the width direction C) of the opening edge 62D.

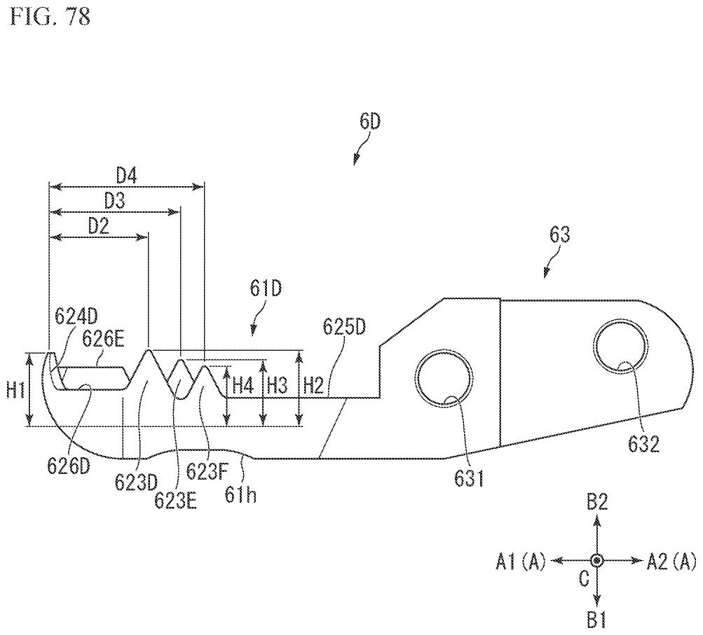

FIG. 78 is a side view of the forceps piece 6D.

The first tooth 624D, the second tooth 623D, the third tooth 623E, and the fourth tooth 623F are arranged in this order from the distal end side A1 toward the proximal side A2 in a side view in the width direction C. A length D2 in the longitudinal direction A from the first tooth 624D to the second tooth 623D is shorter than a length D3 in the longitudinal direction A from the first tooth 624D to the third tooth 623E. A length D3 in the longitudinal direction A from the first tooth 624D to the third tooth 623E is shorter than a length D4 in the longitudinal direction A from the first tooth 624D to the fourth tooth 623F.

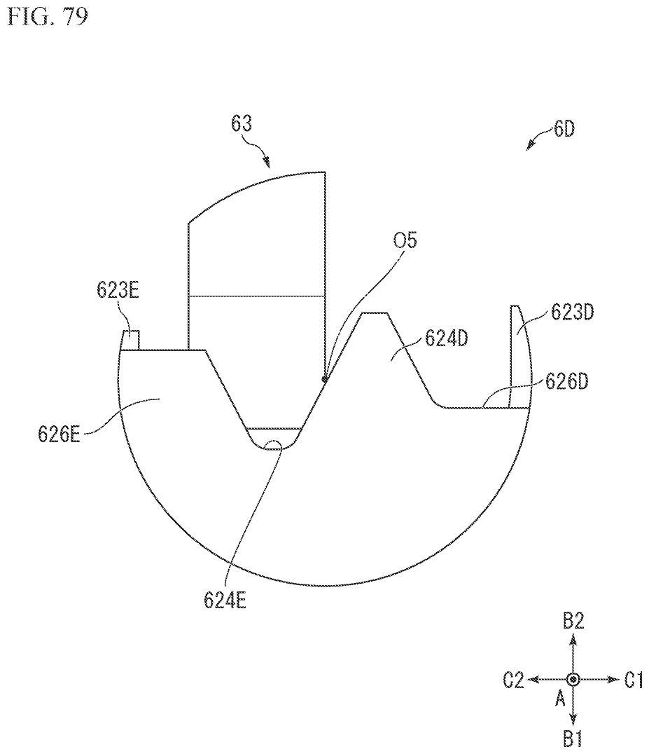

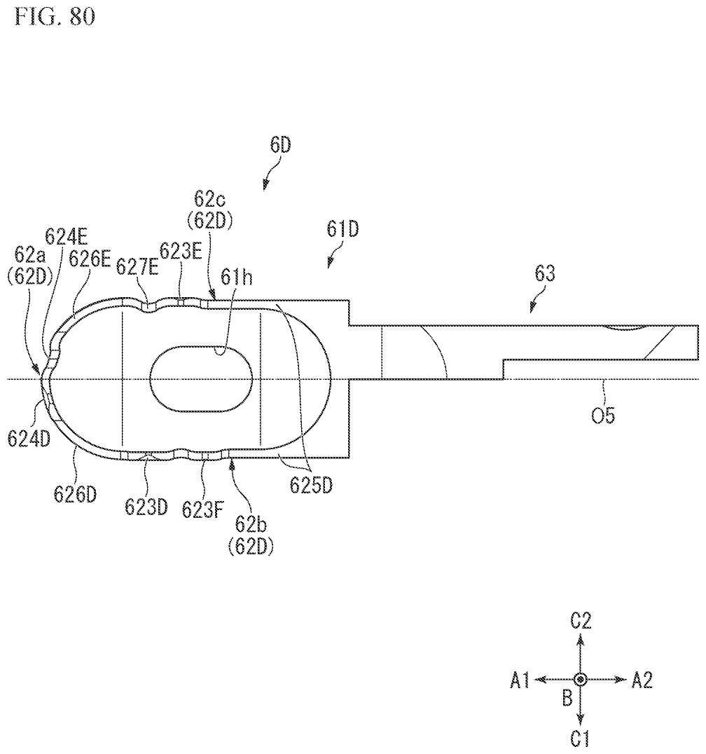

FIG. 79 is a front view of the forceps piece 6D. FIG. 80 is a plan view of the forceps piece 6D.

The first tooth 624D is a tooth protruding to the upper side B2 provided on the distal end side A1 of the opening edge 62D. As shown in FIG. 79, the first tooth 624D is formed in a substantially triangular shape when viewed from the front. Also, the first tooth 624D is provided on the right side C1 of the central axis O5. The first tooth 624D is formed between the tip groove 624E and the recess 626D at the opening edge 62D.

The recess 626D is a recessed portion recessed to the lower side B1 provided at the leading end edge 62a of the opening edge 62D. The recess 626D is formed between the first tooth 624D and the second tooth 623D at the opening edge 62D.

The tip groove portion 624E is a groove recessed to the lower side B1 provided in the tip edge 62a of the opening edge 62D. As shown in FIG. 79, the tip groove portion 624E is formed in a substantially triangular shape when viewed from the front. Further, the tip groove portion 624E is provided on the left side C2 of the central axis O5. The tip groove portion 624E is formed between the first tooth 624D and the protruding portion 626E at the opening edge 62D.

The protruding portion 626E is a protruding portion protruding to the upper side B2 provided on the leading end edge 62a of the opening edge 62D. As shown in FIG. 75, the protruding portion 626E is formed in a substantially trapezoidal shape when viewed from the side. As shown in FIG. 78, the upper end of the protruding portion 626E is formed on a flat surface and is lower in the vertical direction B than the upper end of the first tooth 624D. The protruding portion 626E is formed between the tip groove portion 624E and the side groove portion 627E at the opening edge 62D.

The side groove portion 627E is a groove recessed to the lower side B1 provided on the second edge 62c of the opening edge 62D. As shown in FIG. 75, the tip groove portion 624E is formed in a substantially triangular shape when viewed from the side. The side groove 627E is formed between the protrusion 626E and the third tooth 623E at the opening edge 62D.

The second tooth 623D is a tooth protruding to the upper side B2 provided on the first edge 62b of the opening edge 62D. As shown in FIG. 78, the second tooth 623D is formed in a substantially triangular shape when viewed from the side. The protruding length H2 (the length protruding from the forceps piece 6D toward the forceps piece 7D) of the upper end (distal end in the vertical direction B) of the second tooth 623D is substantially the same as the protruding length H1 of the upper end of the first tooth 624D. The second tooth 623D is formed between the recess 626D and the fourth tooth 623F at the opening edge 62D.

The third tooth 623E is a tooth protruding to the upper side B2 provided on the second edge 62c of the opening edge 62D. As shown in FIG. 78, the third tooth 623E is formed in a substantially triangular shape when viewed from the side. The protruding length H3 of the upper end of the third tooth 623E is shorter than the protruding length H2 of the upper end of the second tooth 623D. The third tooth 623E is formed between the side groove portion 627E and the pair of flat portions 625D at the opening edge 62D.

The fourth tooth 623F is a tooth protruding to the upper side B2 provided on the first edge 62b of the opening edge 62D. As shown in FIG. 78, the fourth tooth 623F is formed in a substantially triangular shape when viewed from the side. The protruding length H4 of the upper end of the fourth tooth 623F is shorter than the protruding length H3 of the upper end of the third tooth 623E. The fourth tooth 623F is formed between the second tooth 623D and the pair of flat portions 625D at the opening edge 62D. The fourth tooth 623F is provided closer to the distal end side A1 in the longitudinal direction A than the proximal end of the through hole 61h. Specifically, the proximal end of the fourth tooth 623F (the boundary between the end of the fourth tooth 623F on the proximal end side A2 and the pair of flat portions 625D) is provided closer to the distal end side A1 than the proximal end of the through hole 61h.

The pair of flat portions 625D includes a right flat portion formed on the proximal end side A2 of the fourth tooth 623F at the first edge 62b of the opening edge 62D, and a left flat portion formed on the proximal end side A2 of the third tooth 623E at the second edge 62c of the opening edge 62D. The pair of flat portions 625D extend linearly along the central axis O5.

Note that the protruding length H1 of the upper end of the first tooth 624D may be less than or equal to the protruding length H2 of the upper end of the second tooth 623D. Also, the protruding length H1 of the upper end of the first tooth 624D may be slightly shorter than the protruding length H2 of the upper end of the second tooth 623D and longer than the protruding length H3 of the upper end of the third tooth 623E.

[Forceps piece (first forceps piece) 7D]

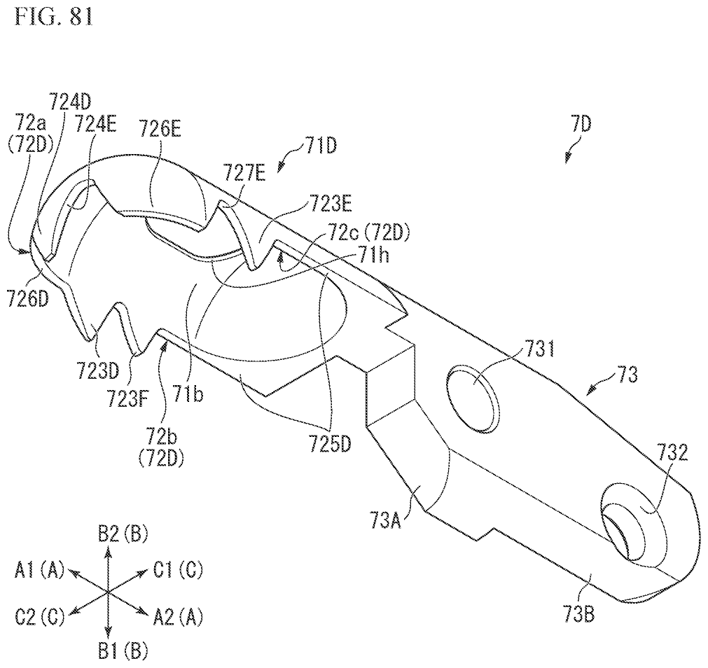

FIG. 81 is a perspective view of the forceps piece 7D.

A forceps piece (first forceps piece) 7D is a member having the same shape as the forceps piece 6D. The forceps piece 7D is formed, for example, from a single flat plate or a single bar by cutting or pressing. The forceps piece 7D has a forceps cup (first forceps cup) 71D provided on the distal end side A1 in the longitudinal direction A, and a second plate 73.

The forceps cup 71D is formed in a substantially hemispherical shape and opens toward the forceps piece 6D side (lower side B1) in the opening/closing direction B. The forceps cup 71D has an opening edge 72D and a through-hole 71h penetrating in the opening/closing direction B near the center of the concave bottom portion 71b.

The opening edge 72D is the opening edge of the forceps cup 71D, and is formed in a substantially arc shape in plan view. The opening edge 72D includes a pair of first teeth 724D, recesses 726D, tip grooves 724E, protruding portions 726E, side grooves 727E, second teeth 723D, third teeth 723E, fourth teeth 723F, and a flat portion 725D.

The opening edge 72D is divided into a leading edge 72a that is an edge on the leading edge side A1 of the opening edge 72D, a first edge 72b that is an edge on the left side C2 (the second side in the width direction C) of the opening edge 72D, and a second edge 72c that is an edge on the right side C1 (the first side in the width direction C) of the opening edge 72D.

The first tooth 724D, the second tooth 723D, the third tooth 723E, and the fourth tooth 723F are arranged in this order from the distal end side A1 toward the proximal side A2 in a side view in the width direction C.

The first tooth 724D is a tooth protruding to the lower side B1 provided on the distal end side A1 of the opening edge 72D. As shown in FIG. 76, the first tooth 724D is formed in a substantially triangular shape when viewed from the front. Also, the first tooth 724D is provided on the left side C2 of the central axis O5. The first tooth 724D is formed between the tip groove 724E and the recess 726D at the opening edge 72D.

The recess 726D is a recessed portion recessed toward the upper side B2 provided at the leading end edge 72a of the opening edge 72D. A recess 726D is formed between the first tooth 724D and the second tooth 723D at the opening edge 72D.

The tip groove portion 724E is a groove recessed to the upper side B2 provided in the tip edge 72a of the opening edge 72D. As shown in FIG. 76, the tip groove portion 724E is formed in a substantially triangular shape when viewed from the front. Further, the tip groove portion 724E is provided on the right side C1 of the central axis O5. The tip groove portion 724E is formed between the first tooth 724D and the protruding portion 726E at the opening edge 72D.

The protruding portion 726E is a protruding portion protruding to the lower side B1 provided on the leading end edge 72a of the opening edge 72D. As shown in FIG. 74, the protruding portion 726E is formed in a substantially trapezoidal shape when viewed from the side. The lower end of the protruding portion 726E is formed on a flat surface and is higher in the vertical direction B than the lower end of the first tooth 724D. The protruding portion 726E is formed between the tip groove portion 724E and the side groove portion 727E at the opening edge 72D.

The side groove portion 727E is a groove recessed toward the upper side B2 provided on the second edge 72c of the opening edge 72D. As shown in FIG. 74, the tip groove portion 724E is formed in a substantially triangular shape when viewed from the side. The side groove portion 727E is formed between the protruding portion 726E and the third tooth 723E at the opening edge 72D.

The second tooth 723D is a tooth protruding to the lower side B1 provided on the first edge 72b of the opening edge 72D. As shown in FIG. 75, the second tooth 723D is formed in a substantially triangular shape when viewed from the side. The protruding length H2 (the length protruding from the forceps piece 7D toward the forceps piece 6D) of the lower end (distal end in the vertical direction B) of the second tooth 723D is substantially the same as the protruding length H1 of the lower end of the first tooth 724D. The second tooth 723D is formed between the recess 726D and the fourth tooth 723F at the opening edge 72D.

The third tooth 723E is a tooth protruding to the lower side B1 provided on the second edge 72c of the opening edge 72D. As shown in FIG. 74, the third tooth 723E is formed in a substantially triangular shape when viewed from the side. The protruding length H3 of the lower end of the third tooth 723E is shorter than the protruding length H2 of the lower end of the second tooth 723D. The third tooth 723E is formed between the side groove portion 727E and the pair of flat portions 725D at the opening edge 72D.

The fourth tooth 723F is a tooth protruding to the lower side B1 provided on the first edge 72b of the opening edge 72D. As shown in FIG. 75, the fourth tooth 723F is formed in a substantially triangular shape when viewed from the side. The protruding length H4 of the lower end of the fourth tooth 723F is shorter in the vertical direction B than the protruding length H3 of the lower end of the third tooth 723E. The fourth tooth 723F is formed between the second tooth 723D and the pair of flat portions 725D at the opening edge 72D. The fourth tooth 723F is provided closer to the distal end side A1 in the longitudinal direction A than the proximal end of the through hole 71h. Specifically, the proximal end of the fourth tooth 723F (the boundary between the end of the fourth tooth 723F on the proximal end side A2 and the pair of flat portions 725D) is provided closer to the distal end side A1 than the proximal end of the through hole 71h.

The pair of flat portions 725D includes a left flat portion formed on the proximal end side A2 of the fourth tooth 723F at the first edge 72b of the opening edge 72D, and a right flat portion formed on the proximal end side A2 of the third tooth 723E at the second edge 72c of the opening edge 72D. The pair of flat portions 725D extend linearly along the central axis O5.

It should be noted that the protruding length H1 of the lower end of the first tooth 724D may be less than or equal to the protruding length H2 of the lower end of the second tooth 723D. Also, the protruding length H1 of the lower end of the first tooth 724D may be slightly shorter than the protruding length H2 of the lower end of the second tooth 723D and longer than the protruding length H3 of the lower end of the third tooth 723E.

[Forceps piece 6D and forceps piece 7D]

When the forceps 5D is closed, as shown in FIG. 76, the first tooth 624D of the forceps piece 6D engages with the tip groove 724E of the forceps piece 7D, and the first tooth 724D of the forceps piece 7D engages with the tip groove 624E of the forceps piece 6D.

When the forceps 5D is closed, as shown in FIG. 74, the protrusion 726E of the forceps piece 7D engages with the recess 626D of the forceps piece 6D. Also, the second tooth 623D of the forceps piece 6D engages with the side groove 727E of the forceps piece 7D. The third tooth 723E of the forceps piece 7D is positioned in the longitudinal direction A between the second tooth 623D and the fourth tooth 623F of the forceps piece 6D.

When the forceps 5D is closed, the length G1 (see FIG. 76) of the gap in the vertical direction B between the upper end of the first tooth 624D and the tip groove 724E is substantially the same as the length G2 (see FIG. 74) of the gap in the vertical direction B between the upper end of the second tooth 623D and the side groove portion 727E. When the forceps 5D are closed, the gap length G2 is smaller than the gap length G4 (see FIG. 74) in the vertical direction B between the upper end of the fourth tooth 623F and the pair of flat portions 725D.

When the forceps 5D is closed, as shown in FIG. 75, the protruding portion 626E of the forceps piece 6D abuts and engages with the recess 726D of the forceps piece 7D. Also, the second tooth 723D of the forceps piece 7D engages with the side groove 627E of the forceps piece 6D. The third tooth 623E of the forceps piece 6D is positioned in the longitudinal direction A between the second tooth 723D and the fourth tooth 723F of the forceps piece 7D.

When the forceps 5D is closed, the length G1 (see FIG. 76) of the gap in the vertical direction B between the lower end of the first tooth 724D and the tip groove 624E is substantially the same as the length G2 (see FIG. 75) of the gap in the vertical direction B between the lower end of the second tooth 723D and the side groove portion 627E. “The length G1 is substantially the same as the length G2” means that the difference between G1 and G2 is within 0.05 mm or within 10% of G1 or G2. When the forceps 5D are closed, the gap length G2 is smaller than the gap length G4 (see FIG. 75) in the vertical direction B between the lower end of the fourth tooth 723F and the pair of flat portions 625D.

When the forceps 5D is closed, as shown in FIGS. 74 and 75, the pair of flat portions 625D of the forceps piece 6D and the flat portion 725D of the forceps piece 7D face each other in the vertical direction B, and a predetermined gap E1 is formed therebetween.

[Operating portion 8]

The operating portion 8 is provided on the proximal side A2 of the sheath 1. The operating portion 8 includes an operating portion main body 81, a main body lid 82 and a slider 83. The main body lid 82 is attachable to and detachable from the operating portion main body 81. The proximal end 1b of the sheath 1 is fixed to the distal end portion of the operating portion main body 81 by attaching the main body lid 82 to the operating portion main body 81.

The slider 83 is attached to the operating portion main body 81 so as to be movable along the longitudinal direction A. A proximal end of the operation wire 2 is fixed to the slider 83. The operation wire 2 advances and retreats when the user advances and retreats the slider 83 relative to the operating portion main body 81.

[Action of treatment instrument 100D]

The user brings the forceps 5D of the treatment instrument 100D inserted into the treatment instrument insertion channel 216 of the endoscope 200 closer to the biological tissue T to be collected. The user advances the slider 83 of the operating portion 8 to advance the operation wire 2 and open the forceps 5D.

The first tooth 624D and the first tooth 724D provided on the distal end side A1 of the forceps 5D have relatively short protruding lengths protruding in directions facing each other. Specifically, the protruding length of the first tooth 624D protruding toward the forceps piece 7D is substantially equal to that of the second tooth 623D. The protruding length of the first tooth 724D protruding toward the forceps piece 6D is substantially equal to that of the second tooth 723D. Therefore, when the forceps 5D are opened, the range of the forceps 5D to grasp the living tissue T is wide.

The user brings the open forceps 5D into contact with the biological tissue T to be collected. The user retracts the slider 83 of the operating portion 8 to retract the operation wire 2 and close the forceps 5D.

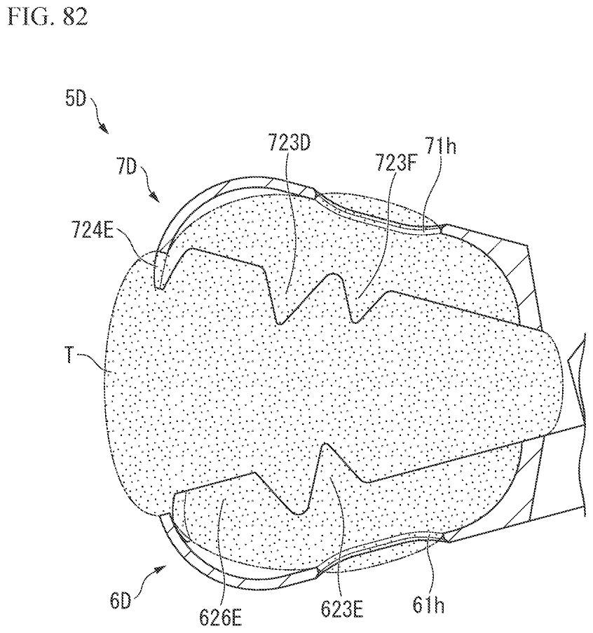

FIG. 82 is a cross-sectional view of the forceps 5D holding the living tissue T.

The second tooth 623D provided on the right side C1 of the forceps 5D and the second tooth 723D provided on the left side C2 of the forceps 5D are provided on the distal end side A1 of the forceps 5D, and have a longer protruding length that protrudes in the direction facing each other compared to the other teeth provided on the proximal end side A2. Therefore, the forceps 5D can firmly grasp the living tissue T on the distal end side A1 by the second teeth 623D and the second teeth 723D, and the amount of the living tissue T to be collected is large.

The fourth tooth 623F provided on the right side C1 of the forceps 5D and the fourth tooth 723F provided on the left side C2 of the forceps 5D have a shorter protruding length that protrudes in the direction facing each other compared to the other teeth provided on the distal end side A1. Therefore, when the living tissue T is grasped by the other teeth on the distal end side A1, the gripping force of the living tissue T is concentrated on the other teeth on the distal end side A1, so that the living tissue T does not slip easily.

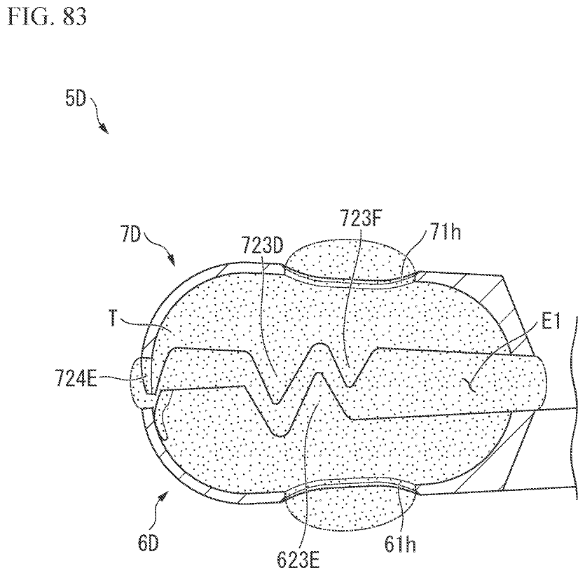

FIG. 83 is a cross-sectional view of the forceps 5D holding the living tissue T.

The fourth tooth 623F and the fourth tooth 423F are provided closer to the distal end side A1 than the proximal end of the through hole 61h and the through hole 71h in the longitudinal direction A. Therefore, even when the living tissue T grasped by the forceps 5D is large, the forceps 5D can easily grasp the living tissue T in a state in which a part of the living tissue T protrudes outward from the through holes 61h and 71h, and a large amount of tissue can be collected.

Since the forceps 5D can release the living tissue T sandwiched by the forceps 5D from the gap E1, it is possible to extract a large amount of tissue.

The forceps 5D has teeth, protruding portions, and the like asymmetrically provided with respect to the vertical plane VP passing through the central axis O3, and can grasp the biological tissue T while twisting it intricately. Therefore, it is possible to prevent the living tissue T from slipping from the forceps 5D when the living tissue T is grasped or torn off.

According to the endoscopic treatment instrument 100D according to the present embodiment, it is possible to suppress the living tissue T from slipping from the forceps 5D when collecting the living tissue T, so that a large amount of the living tissue T can be collected. Since the forceps piece 6D and the forceps piece 7D are members having the same shape, the endoscope treatment instrument 100D can reduce the number of parts. The endoscope treatment instrument 100D becomes easier to insert when inserted into the treatment instrument insertion channel 216 of the endoscope 200.

In the above embodiment, the forceps piece 6D and the forceps piece 7D are members having the same shape. However, forceps piece 6D and forceps piece 7D may be members not having the same shape.

### Sixth Embodiment

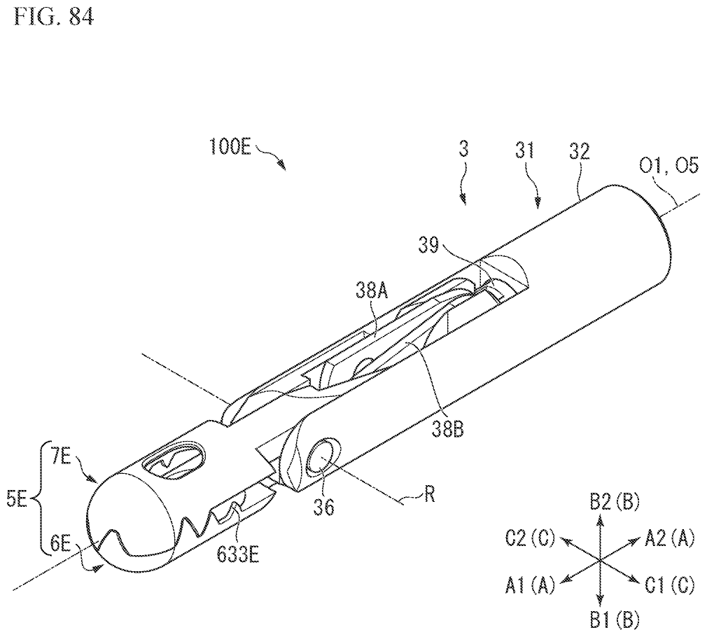

An endoscope treatment instrument 100E according to a sixth embodiment of the present disclosure will be described with reference to FIGS. 84 to 98. In the following description, the same reference numerals are given to the same configurations as those already described, and redundant descriptions will be omitted.

FIG. 84 is a perspective view of the forceps 5E of the endoscopic treatment instrument 100E according to the sixth embodiment.

The endoscope treatment instrument 100E (simply referred to as the treatment instrument 100E) is used as an endoscope treatment system together with the endoscope 200, like the endoscope treatment instrument 100 of the first embodiment. The treatment instrument 100E includes a sheath 1, an operation wire 2, a support member 3, forceps 5E, and an operating portion 8.

[Forceps (jaw) 5E]

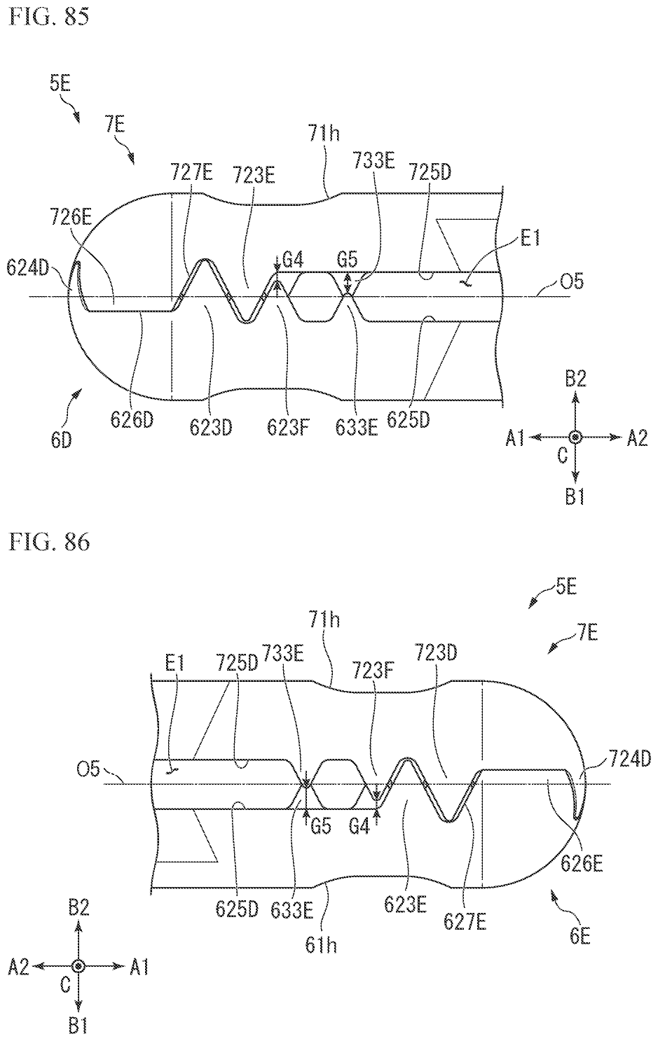

FIG. 85 is a right side view of the forceps 5E. FIG. 86 is a left side view of the forceps 5E.

A forceps (jaw) 5E is a member for collecting a living tissue. The forceps 5E is made of a metal material such as stainless steel, and includes a forceps piece 6E and a forceps piece 7E that face each other in the closed state. When the forceps piece 6E and the forceps piece 7E are closed, the forceps 5E has a cylindrical shape. The forceps piece 6E and the forceps piece 7E are supported by a pin 36 so as to be capable of opening and closing toward the distal end side A1.

The forceps piece 6E is rotatably supported by the pin 36 with the rotation axis R of the pin 36 as the center of rotation. The forceps piece 6E is connected to the connection portion 39 via the support plate member 38A at the proximal end side A2 and is operated by the operation wire 2 connected to the connection portion 39.

The forceps piece 7E is rotatably supported by the pin 36 with the rotation axis R of the pin 36 as the center of rotation. The forceps piece 7E is connected to the connection portion 39 via the support plate member 38B at the proximal end side A2, and is operated by the operation wire 2 connected to the connection portion 39.

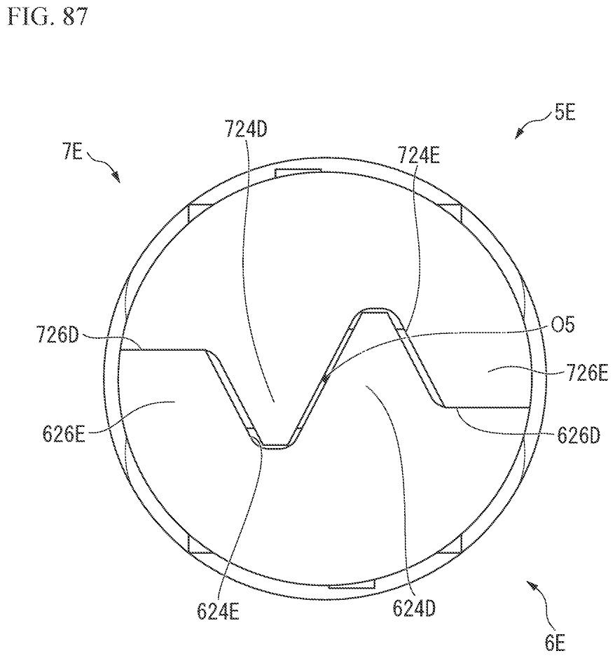

FIG. 87 is a front view of the forceps 5E.

The forceps piece 6E and the forceps piece 7E are arranged symmetrically with respect to the central axis O5 in the longitudinal direction A of the forceps 5E. A central axis O5 in the longitudinal direction A of the forceps 5E substantially coincides with a central axis O1 in the longitudinal direction A of the sheath 1.

One of the forceps piece 6E and the forceps piece 7E may be fixed to the support member 3, and only the other may be rotatably supported by the support member 3.

[Forceps piece (second forceps piece) 6E]

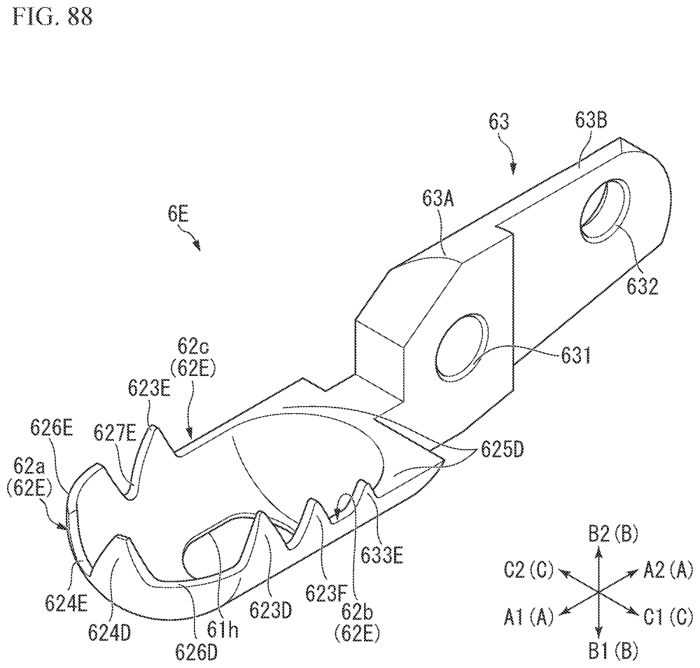

FIG. 88 is a perspective view of the forceps piece 6E.

The forceps piece (second gripping piece) 6E is formed, for example, mainly by cutting or pressing from one flat plate or one bar. The forceps piece 6E has a forceps cup (second forceps cup) 61E provided on the distal end side A1 in the longitudinal direction A, and a first plate 63.

The forceps cup 61E is formed in a substantially hemispherical shape and opens toward the forceps piece 7E side (upper side B2) in the opening/closing direction B. The forceps cup 61E has an opening edge 62E and a through-hole 61h penetrating in the opening/closing direction B near the center of the concave bottom portion 61b.

The opening edge 62E is the opening edge of the forceps cup 61E, and is formed in a substantially arc shape in plane view. The opening edge 62E includes a first tooth 624D, a recess 626D, a tip groove 624E, a protruding portion 626E, a side groove 627E, a second tooth 623D, a third tooth 623E, a fourth tooth 623F, a fourth tooth 623F, a fifth tooth 633E, and a pair of flat portions 625D.

The opening edge 62E is divided into a leading end edge 62a that is the edge of the leading end side A1 of the opening edge 62E, a first edge 62b that is the edge of the right side C1 (the first side in the width direction C) of the opening edge 62E, and a second edge 62c that is an edge on the left side C2 (the second side in the width direction C) of the opening edge 62E.

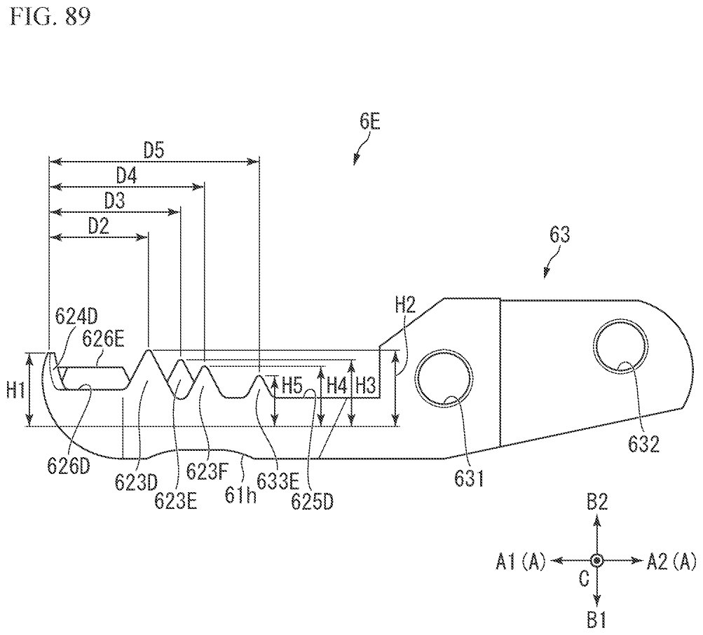

FIG. 89 is a side view of the forceps piece 6E.

The length D4 from the first tooth 624D to the fourth tooth 623F in the longitudinal direction A is shorter than the length D5 from the first tooth 624D to the fifth tooth 633E in the longitudinal direction A.

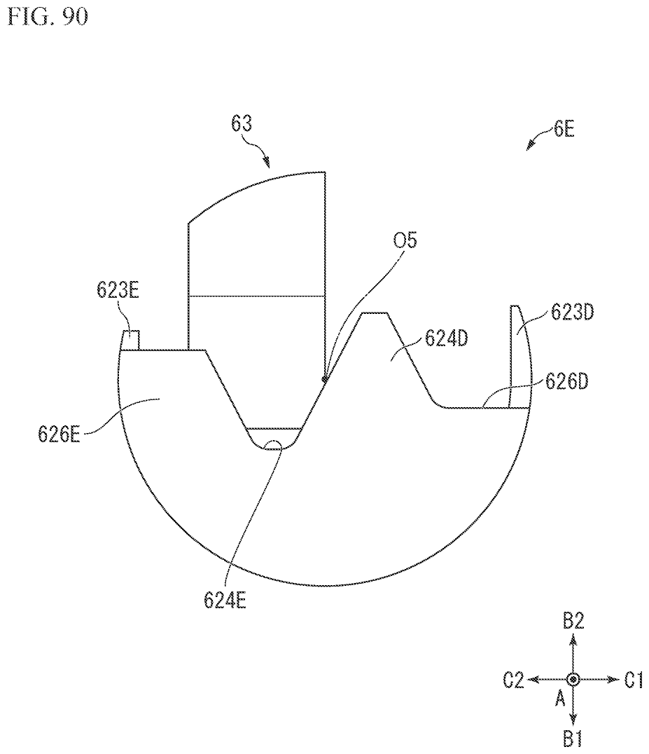

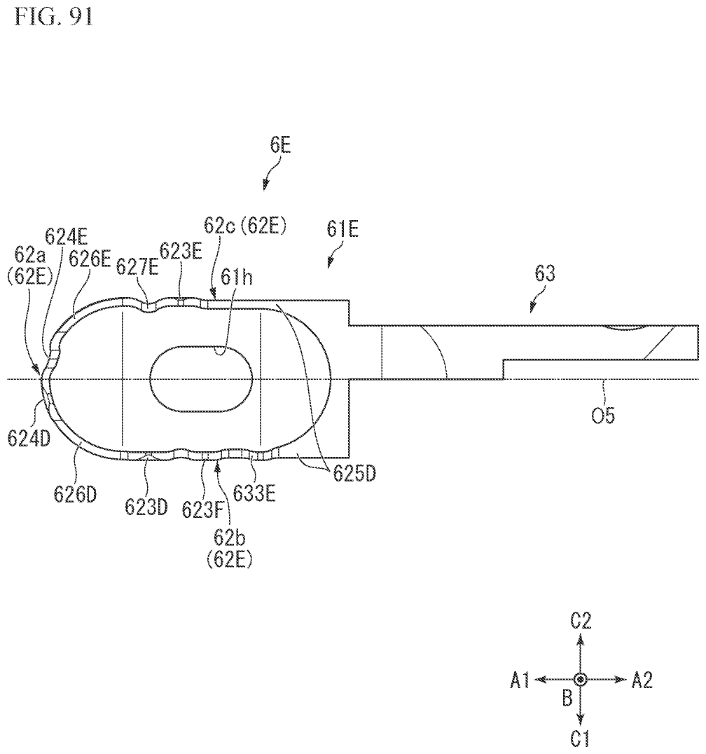

FIG. 90 is a front view of the forceps piece 6E. FIG. 91 is a plan view of the forceps piece 6E.

The first tooth 624D is a tooth protruding to the upper side B2 provided on the distal end side A1 of the opening edge 62E. As shown in FIG. 90, the first tooth 624D is formed in a substantially triangular shape when viewed from the front. Also, the first tooth 624D is provided on the right side C1 of the central axis O5. The first tooth 624D is formed between the tip groove 624E and the recess 626D at the opening edge 62D.

The fifth tooth 633E is a tooth protruding to the upper side B2 provided on the first edge 62b of the opening edge 62E. As shown in FIG. 89, the fifth tooth 633E is formed in a substantially triangular shape when viewed from the side. The protruding length H5 of the upper end of the fifth tooth 633E is shorter than the protruding length H4 of the upper end of the fourth tooth 623F. The fifth tooth 633E is formed between the fourth tooth 623F and the pair of flat portions 625D at the opening edge 62E. The fifth tooth 633E and the tooth provided on the forceps piece 7E are arranged apart in the longitudinal direction A without engaging with each other.

[Forceps piece (first forceps piece) 7E]

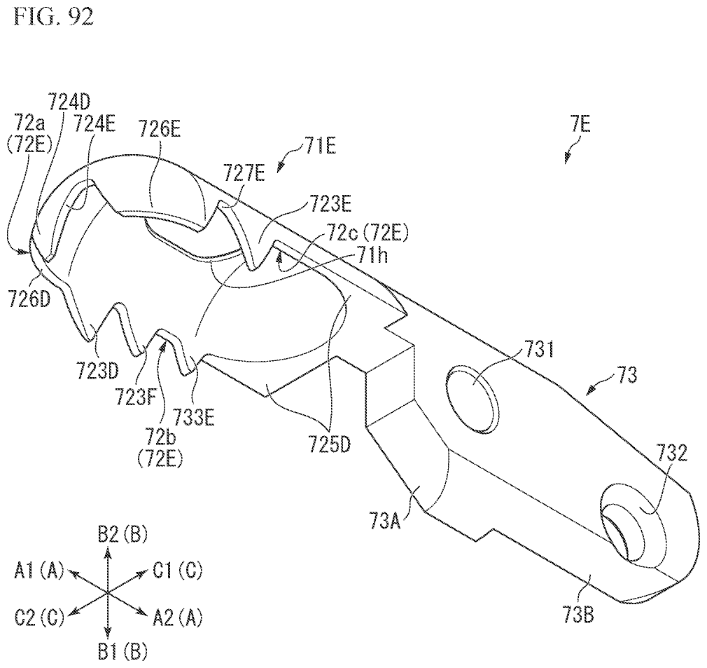

FIG. 92 is a perspective view of the forceps piece 7E.

The forceps piece (first forceps piece) 7E is a member having the same shape as the forceps piece 6E. The forceps piece 7E is formed, for example, from a single flat plate or a single bar by cutting or pressing. The forceps piece 7E has a forceps cup (first forceps cup) 71E provided on the distal end side A1 in the longitudinal direction A, and a second plate 73.

The forceps cup 71E is formed in a substantially hemispherical shape and opens toward the forceps piece 6E side (lower side B1) in the opening/closing direction B. The forceps cup 71E has an opening edge 72E and a through-hole 71h penetrating in the opening/closing direction B near the center of the concave bottom portion 71b.

The opening edge 72E is the opening edge of the forceps cup 71E and is formed in a substantially arc shape in plane view. The opening edge 72E includes a first tooth 724D, a recess 726D, a tip groove 724E, a protrusion 726E, a side groove 727E, a second tooth 723D, a third tooth 723E, a fourth tooth 723F, a fourth tooth 723F, a fifth tooth 733E, and a pair of flat portions 725D.

The opening edge 72E is divided into a leading edge 72a that is an edge on the leading edge side A1 of the opening edge 72E, a first edge 72b that is an edge on the left side C2 (the second side in the width direction C) of the opening edge 72E, and a second edge 72c that is an edge on the right side C1 (the first side in the width direction C) of the opening edge 72E.

The fifth tooth 733E is a tooth protruding to the lower side B1 provided on the first edge 72b of the opening edge 72E. As shown in FIG. 86, the fifth tooth 733E is formed in a substantially triangular shape when viewed from the side. The protruding length H5 of the upper end of the fifth tooth 733E is shorter than the protruding length H4 of the upper end of the fourth tooth 723F. The fifth tooth 733E is formed between the fourth tooth 723F and the pair of flat portions 725D at the opening edge 72E. The fifth tooth 733E and the tooth provided on the forceps piece 6E are arranged apart in the longitudinal direction A without engaging with each other.

[Forceps piece 6E and forceps piece 7E]

When the forceps 5E are closed, the length G4 (see FIG. 85) of the gap in the vertical direction B between the upper end of the fourth tooth 623F and the pair of flat portions 725D is smaller than the gap length G5 (see FIG. 85) in the vertical direction B.

When the forceps 5E are closed, the length G4 (see FIG. 86) of the gap in the vertical direction B between the upper end of the fourth tooth 723F and the pair of flat portions 625D is smaller than the gap length G5 (see FIG. 86) in the vertical direction B.

[Action of treatment instrument 100E]

The user brings the forceps 5E of the treatment instrument 100E inserted into the treatment instrument insertion channel 216 of the endoscope 200 closer to the biological tissue T to be collected. The user advances the slider 83 of the operating portion 8 to advance the operation wire 2 and open the forceps 5E. The user brings the open forceps 5E into contact with the biological tissue T to be collected. By retracting the slider 83 of the operating portion 8, the user retracts the operation wire 2 and closes the forceps 5E.

The fifth tooth 633E and the teeth provided on the forceps piece 7E are arranged apart in the longitudinal direction A without engaging with each other. In addition, the fifth tooth 733E and the teeth provided on the forceps piece 6E are arranged apart in the longitudinal direction A without engaging with each other. Therefore, when the living tissue T is gripped by the other tooth on the distal end side A1, the gripping force of the living tissue T is concentrated on the other tooth on the distal end side A1, so that the living tissue T does not slip easily.

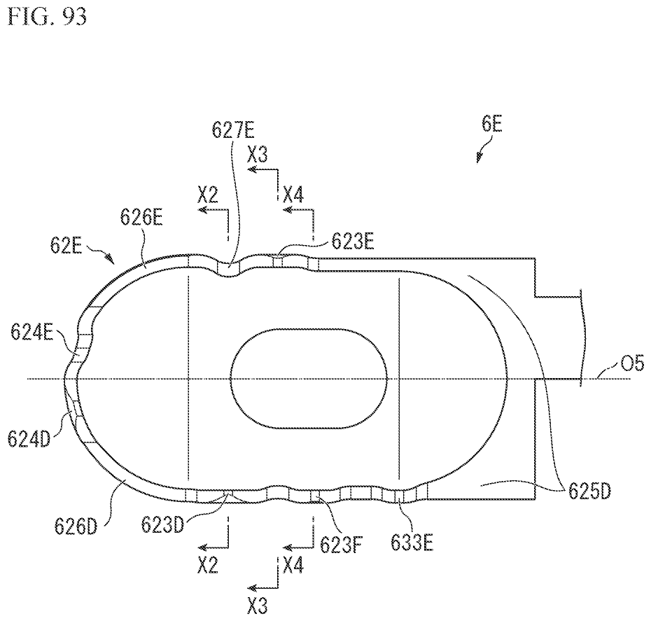

FIG. 93 is a plan view of the forceps piece 6E.

The forceps piece 6E and the forceps piece 7E are provided with teeth, protrusions, and the like asymmetrically with respect to the vertical plane VP passing through the central axis O5, and can grasp the living tissue T while being twisted in a complicated manner.

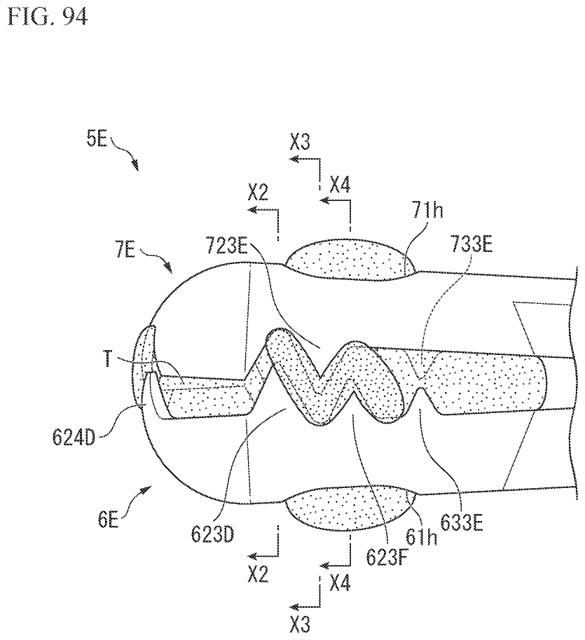

FIG. 94 is a right side view of the forceps 5E holding the living tissue T.

In the forceps 5E, the second teeth 623D, the third teeth 723E, and the fourth teeth 623F are arranged alternately along the longitudinal direction A on the right side C1 of the forceps 5E, and the living tissue T can be grasped while being twisted in the vertical direction B. Specifically, as shown in FIG. 94, the forceps 5E can grasp the living tissue T at the right side C1 of the forceps 5E while twisting it into an N-shape in side view.

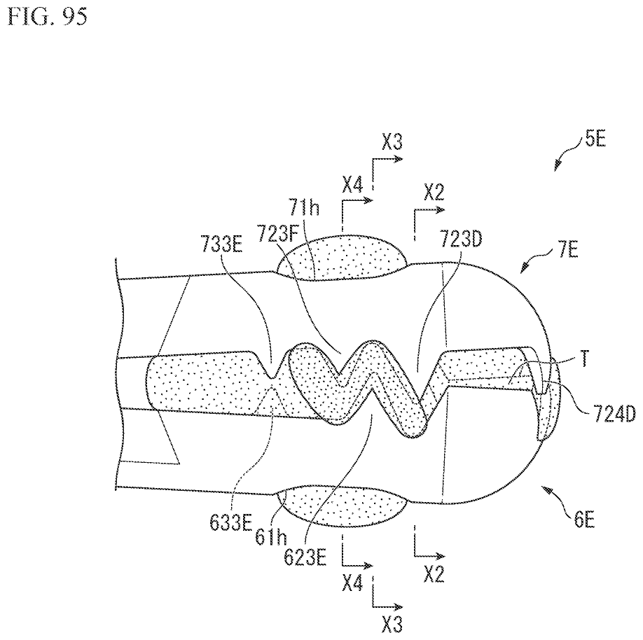

FIG. 95 is a right side view of the forceps 5E holding the living tissue T.

In the forceps 5E, the second teeth 723D, the third teeth 623E, and the fourth teeth 723F are arranged alternately along the longitudinal direction A on the left side C2 of the forceps 5E, and the living tissue T can be grasped while being twisted in the vertical direction B. Specifically, as shown in FIG. 95, the forceps 5E can grasp the living tissue T while twisting it into an N-shape in a side view at the left side C2 of the forceps 5E.

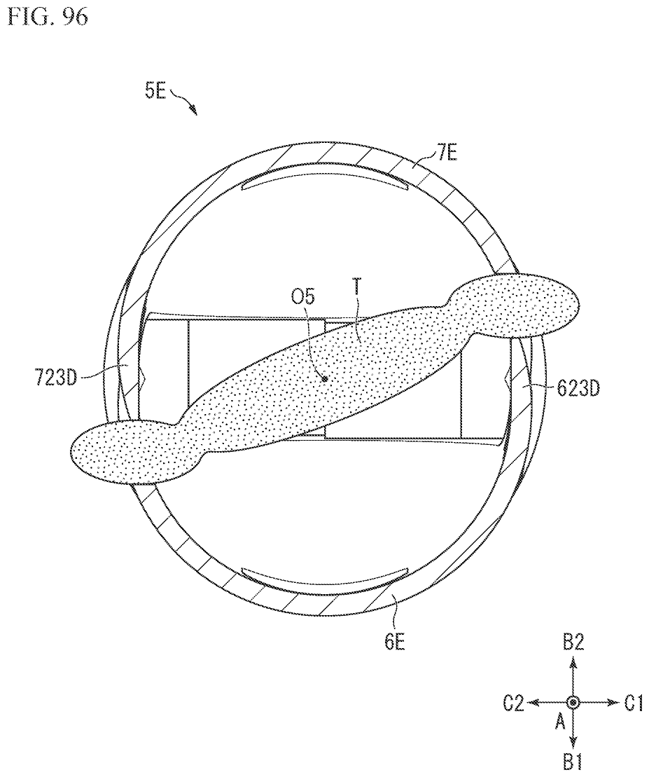

FIG. 96 is a cross-sectional view along X2 shown in FIGS. 93 to 95.

In the cross section along X2, the second teeth 623D of the forceps piece 6E and the second teeth 723D of the forceps piece 7E press the living tissue T. The tip of the second tooth 623D and the tip of the second tooth 723D are arranged at symmetrical positions with respect to the central axis O5. Therefore, the right side C1 of the living tissue T is pressed to the upper side B2 from the horizontal plane HP including the central axis O5, and the left side C2 of the living tissue T is pressed to the lower side B1 from the horizontal plane HP including the central axis O5.

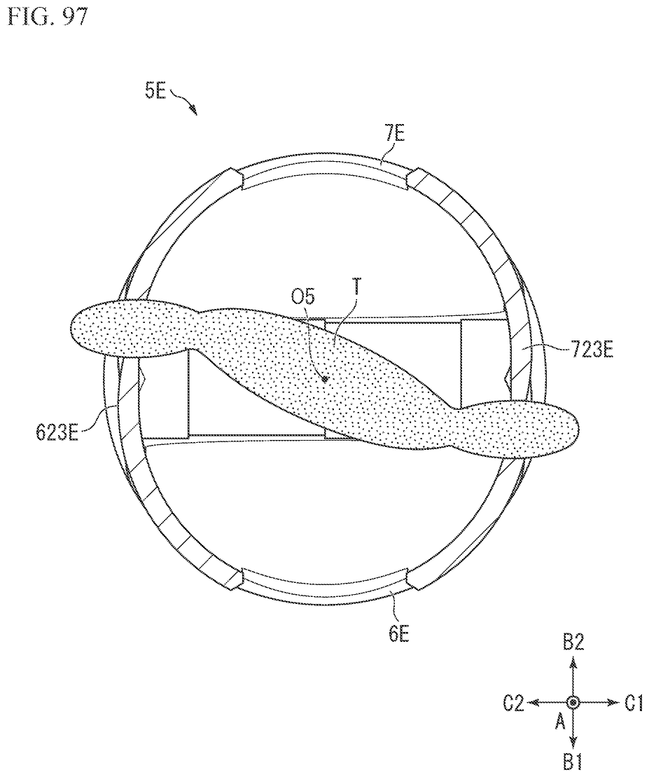

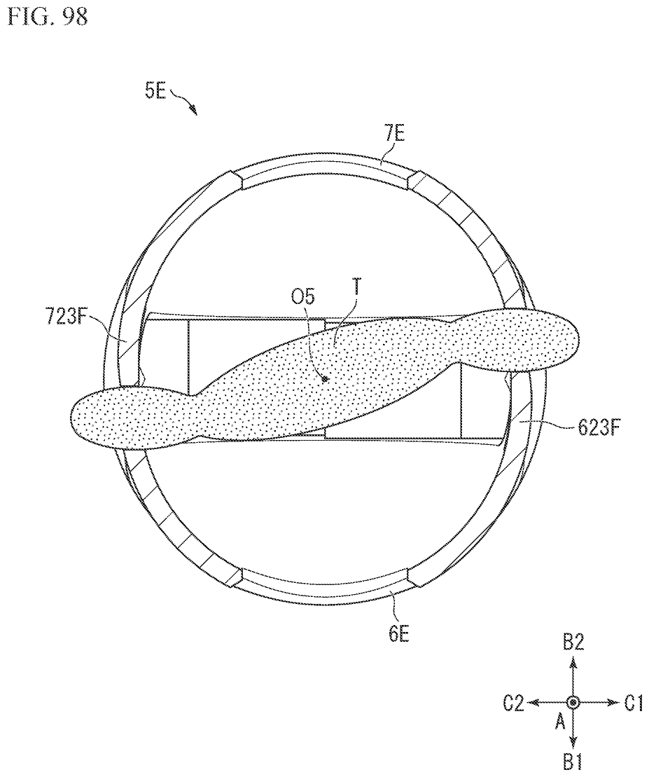

FIG. 97 is a cross-sectional view along X3 shown in FIGS. 93 to 95.

In the cross section along X3, the third tooth 623E of the forceps piece 6E and the third tooth 723E of the forceps piece 7E press the living tissue T. The tip of the third tooth 623E and the tip of the third tooth 723E are arranged at symmetrical positions with respect to the central axis O5. Therefore, the right side C1 of the living tissue T is pressed to the lower side B1 from the horizontal plane HP including the central axis O5, and the left side C2 of the living tissue T is pressed to the upper side B2 from the horizontal plane HP including the central axis O5.

FIG. 98 is a cross-sectional view along X4 shown in FIGS. 93 to 95.

In the cross section along X2, the fourth tooth 623F of the forceps piece 6E and the fourth tooth 723F of the forceps piece 7E press the living tissue T. The tip of the fourth tooth 623F and the tip of the fourth tooth 723F are arranged at symmetrical positions with respect to the central axis O5. Therefore, the right side C1 of the living tissue T is pressed to the upper side B2 from the horizontal plane HP including the central axis O5, and the left side C2 of the living tissue T is pressed to the lower side B1 from the horizontal plane HP including the central axis O5.

The forceps 5E can grasp the biological tissue T while intricately twisting it in the vertical direction B and the horizontal direction C as described above. Therefore, it is possible to prevent the living tissue T from slipping from the forceps 5E when the living tissue T is grasped or torn off

According to the endoscopic treatment instrument 100E according to the present embodiment, it is possible to suppress the living tissue T from slipping from the forceps 5E when collecting the living tissue T, so that a large amount of the living tissue T can be collected. Since the forceps piece 6E and the forceps piece 7E are members having the same shape, the endoscope treatment instrument 100E can reduce the number of parts. The endoscopic treatment instrument 100E becomes easier to insert when inserted into the treatment instrument insertion channel 216 of the endoscope 200.

In the above embodiment, the forceps piece 6E and the forceps piece 7E are members having the same shape. However, the forceps piece 6E and the forceps piece 7E may be members not having the same shape.

Although the first to sixth embodiments of the present disclosure have been described in detail with reference to the drawings, the specific configuration is not limited to these embodiments, and design changes and the like within a range that does not deviate from the gist of the present disclosure are also included. In addition, the components shown in the above-described embodiments and modifications can be appropriately combined and configured.

### Modification Example

The forceps (the first forceps piece and the second forceps piece) in the above embodiment may be formed in a shape in which they are left-right reversed with respect to the vertical plane VP including the central axis O5.

### Modification Example

In the above embodiment, the endoscopic treatment tool is a biopsy forceps, but the endoscopic treatment tool is not limited to this. The endoscopic treatment tool may be, for example, a grasping forceps having a pair of forceps pieces as well.

### INDUSTRIAL APPLICABILITY

The present disclosure can be applied to endoscopic treatment tools.

What is claimed is:

1. An endoscope treatment tool, comprising:
a forceps having a first forceps piece and a second forceps piece; and
a joint
wherein the first forceps piece and the second forceps piece are connected at the joint for relative motion to open and close the forceps,
wherein the second forceps piece includes:
a forceps cup opening toward the first forceps piece, wherein the forceps cup has an opening edge having a first edge and a second edge, wherein the first edge is on a first side in a width direction of the opening edge and the second edge is on a second side in the width direction of the opening edge,
a first tooth provided on a tip portion of the opening edge,
a second tooth provided on the first edge,
a third tooth provided on the second edge, and
a fourth tooth provided on the first edge,
wherein a length from a tip of the first tooth to a tip of the second tooth in a longitudinal direction defines a first length, a length from the tip of the first tooth to a tip of the third tooth in the longitudinal direction defines a second length, and the first length is shorter than the second length, and
wherein a length from the tip of the first tooth to a tip of the fourth tooth in the longitudinal direction defines a third length and the second length is shorter than the third length.

2. The endoscopic treatment tool according to claim 1, wherein a second imaginary line between a frontmost part of the second tooth and a rearmost part of the second tooth defines a second base, a third imaginary line between a frontmost part of the third tooth and a rearmost part of the third tooth defines a third base, and a fourth imaginary line between a frontmost part of the fourth tooth and a rearmost part of the fourth tooth defines a fourth base
wherein a length from the second base to the tip of the second tooth defines a second protruding length, a length from the third base to the tip of the third tooth defines a third protruding length, and the second protruding length is longer than the third protruding length, and
wherein a length from the fourth base to the tip of the fourth tooth defines a fourth protruding length and the third protruding length is longer than the fourth protruding length.

3. The endoscopic treatment tool according to claim 1, wherein a first imaginary line between a frontmost part of the first tooth and a rearmost part of the first tooth defines a first base and a second imaginary line between a frontmost part of the second tooth and a rearmost part of the second tooth defines a second base, and
wherein a length from the first base to the tip of the first tooth defines a first protruding length and a length from the second base to the tip of the second tooth defines a second protruding length, and the first protruding length is shorter than or equal to the second protruding length.

4. The endoscopic treatment tool according to claim 1, wherein the second forceps piece further includes a surface located on the opening edge between the first tooth and the second tooth.

5. The endoscopic treatment tool according to claim 4, wherein no other tooth is located on the surface located on the opening edge between the first tooth and the second tooth.

6. The endoscopic treatment tool according to claim 1, wherein the second tooth is adjacent to the fourth tooth at the opening edge, and
wherein a surface of the second tooth abuts a surface of the fourth tooth.

7. The endoscopic treatment tool according to claim 1, wherein the first tooth is provided on the first side of a central longitudinal axis of the forceps, and wherein the tip of the first tooth is in the first edge of the opening edge.

8. The endoscopic treatment tool according to claim 1, wherein a portion of the first edge on a proximal side of the fourth tooth has a first flat surface and a portion of the second edge on a proximal side of the third tooth has a second flat surface, and
wherein, when the first forceps piece and the second forceps piece are in a closed configuration, the first flat surface forms a first gap between the first forceps piece and the second forceps piece, and the second flat surface forms a second gap between the first forceps piece and the second forceps piece.

9. The endoscopic treatment tool according to claim 8, wherein the first gap and the second gap are each sized to pass living tissue between the first forceps piece and the second forceps piece.

10. The endoscopic treatment tool according to claim 1, wherein the forceps cup has a through hole from a first side to a second side that is oriented in an opening/closing direction of the forceps, and
wherein, in the longitudinal direction, the fourth tooth is located closer to a distal end of the forceps cup than a proximal end of the through hole.

11. The endoscopic treatment tool according to claim 1, wherein, when the forceps are closed, a length in an opening/closing direction of the forceps of a gap between a tip of the second tooth and the first forceps piece defines a fifth length,
wherein, when the forceps are closed, a length in an opening/closing direction of the forceps of a gap between a tip of the fourth tooth and the first forceps piece defines a seventh length, and
wherein the fifth length is shorter than the seventh length.

12. The endoscopic treatment tool according to claim 1, wherein, when the forceps are closed, a length in an opening/closing direction of the forceps of a gap between a tip of the first tooth and the first forceps piece defines a fourth length,
wherein, when the forceps are closed, a length in an opening/closing direction of the forceps of a gap between a tip of the second tooth and the first forceps piece defines a fifth length, and
wherein the fourth length is approximately the same as the fifth length

13. The endoscopic treatment tool according to claim 1, wherein the forceps cup of the second forceps piece is a second forceps cup and the opening edge of the second forceps cup is a second opening edge, and
wherein the first forceps piece includes:
a first forceps cup opening toward the second forceps piece, wherein the first forceps cup has a first opening edge having a third edge and a fourth edge, wherein the third edge is on a first side in the width direction of the first opening edge and the fourth edge is on a second side in the width direction of the first opening edge,
a first tooth provided on a tip portion of the first opening edge,
a second tooth provided on the fourth edge,
a third tooth provided on the third edge, and
a fourth tooth provided on the fourth edge.

14. The endoscopic treatment tool according to claim 13, wherein, when the forceps are closed:
the third tooth of the second forceps piece is positioned between the second tooth and the fourth tooth of the first forceps piece in the longitudinal direction, and
the third tooth of the first forceps piece is positioned between the second tooth and the fourth tooth of the second forceps piece in the longitudinal direction.

15. The endoscopic treatment tool according to claim 13, wherein, when the forceps are closed:
the first tooth of the second forceps piece and the first tooth of the first forceps piece are adjacent to each other,
the first tooth of the second forceps piece is located on the first side of the second forceps piece, and
the first tooth of the first forceps piece is located on the second side of the second forceps piece.

16. The endoscopic treatment tool according to claim 1, wherein the second forceps piece further includes a fifth tooth provided on the first edge, and
wherein the fifth tooth is closer to a proximal side of the second forceps piece than the fourth tooth.

17. The endoscopic treatment tool according to claim 16, wherein a fourth imaginary line between a frontmost part of the fourth tooth and a rearmost part of the fourth tooth defines a fourth base and a third imaginary line between a frontmost part of the third tooth and a rearmost part of the third tooth defines a third base,
wherein a length from a fourth base to a tip of the fourth tooth defines a fourth protruding length and a length from a third base to a tip of the third tooth defines a third protruding length and the third protruding length is longer than the fourth protruding length,
wherein a fifth imaginary line between a frontmost part of the fifth tooth and a rearmost part of the fifth tooth defines a fifth base, and
wherein a length from a fifth base to a tip of the fifth tooth defines a fifth protruding length and the fourth protruding length is longer than the fifth protruding length.

18. The endoscopic treatment tool according to claim 16, wherein, when the forceps are closed:
a length of a gap between a tip of the second tooth and the first forceps piece in the opening/closing direction of the forceps defines a second gap length,
a length of a gap between a tip of the fourth tooth and the first forceps piece in the opening/closing direction of the forceps defines a fourth gap length,
a length of a gap between a tip of the fifth tooth and the first forceps piece in the opening/closing direction of the forceps defines a fifth gap length, and
the second gap length is smaller than the fourth gap length and the fourth gap length is smaller than the fifth gap length.

19. The endoscopic treatment tool according to claim 16, wherein the forceps cup of the second forceps piece is a second forceps cup and the opening edge of the second forceps cup is a second opening edge, and
wherein the first forceps piece includes:
a first forceps cup opening toward the second forceps piece, wherein the first forceps cup has a first opening edge having a third edge and a fourth edge, wherein the third edge is on a first side in the width direction of the first opening edge and the fourth edge is on a second side in the width direction of the first opening edge,
a first tooth provided on a tip portion of the first opening edge,
a second tooth provided on the fourth edge,
a third tooth provided on the third edge, and
a fourth tooth provided on the fourth edge.

20. The endoscopic treatment tool according to claim 19, wherein the first tooth, the second tooth, the third tooth, and the fourth tooth of the first forceps piece define teeth, and
wherein the teeth of the first forceps are spaced apart in the longitudinal direction, and
wherein, when the forceps are closed, the fifth tooth of the second forceps piece is not located between teeth of the first forceps piece.
